(19) United States
(12) Patent Application Publication
(10) Pub. No.: US 2016/0175634 A1
(43) Pub. Date: Jun. 23, 2016

(54) COMPOSITIONS INCLUDING MATRIX AND BIOMATERIAL, USES THEREOF AND METHODS OF USING THE SAME

(71) Applicant: REGENTS OF THE UNIVERSITY OF MINNESOTA, Minneapolis, MN (US)

(72) Inventors: Adi Ish Am Radian, Falcon Heights, MN (US); Jonathan Konstantine Sakkos, Minneapolis, MN (US); Baris Ragip Mutlu, Minneapolis, MN (US); Lawrence Philip Wackett, St. Paul, MN (US); Alptekin Aksan, Minneapolis, MN (US)

(21) Appl. No.: 14/883,053

(22) Filed: Oct. 14, 2015

Related U.S. Application Data
(60) Provisional application No. 62/063,727, filed on Oct. 14, 2014.

(51) Int. Cl. A62D 3/02; C02F 3/34; E04H 4/12; C12N 1/20

(52) U.S. Cl. CPC A62D 3/02; C12N 1/20; C02F 3/348; E04H 4/1209; C02F 2101/306

(57) ABSTRACT

A composition or article that includes a first silica-matrix encapsulated biomaterial, the first silica-matrix encapsulated biomaterial including a first silica matrix and a first biomaterial; and a second silica-matrix encapsulated biomaterial, the second silica-matrix encapsulated biomaterial including a second silica matrix and a second biomaterial, wherein the first silica-matrix encapsulated biomaterial has at least one property that is different than that of the second silica-matrix encapsulated biomaterial, and wherein the first silica-matrix encapsulated biomaterial forms a first layer and the second silica-matrix encapsulated biomaterial forms a second layer, and the first layer is positioned adjacent the second layer.

### PRIORITY

This application claims priority to U.S. Provisional Application Ser. No. 62/063,727, filed Oct. 14, 2014, entitled COMPOSITIONS INCLUDING A SILICA MATRIX AND BIOMATERIAL, METHODS REGARDING THE SAME AND USES THEREOF, the entire disclosure of which is incorporated herein by reference thereto.

### GOVERNMENT FUNDING

This invention was made with government support under IIP-1237754 and CBET-0644784 awarded by the National Science Foundation. The government has certain rights in the invention.

### SUMMARY

This disclosure describes hydrophobic silica-matrix encapsulated biomaterials including a hydrophobic silica matrix and a biomaterial. As well as silica-matrix encapsulated biomaterials that increase degradation of a target component compared with degradation of the target component by a silica-matrix encapsulated biomaterial formed without a hydrophobic moiety containing compound.

Also disclosed is a composition or article that includes a first silica-matrix encapsulated biomaterial, the first silica-matrix encapsulated biomaterial including a first silica matrix and a first biomaterial; and a second silica-matrix encapsulated biomaterial, the second silica silica-matrix encapsulated biomaterial including a second silica matrix and a second biomaterial, wherein the first silica-matrix encapsulated biomaterial has at least one property that is different than that of the second silica-matrix encapsulated biomaterial, and wherein the first silica-matrix encapsulated biomaterial forms a first layer and the second silica-matrix encapsulated biomaterial forms a second layer, and the first layer is positioned adjacent the second layer.

Also disclosed is a method of making a silica-matrix encapsulated biomaterial for adsorbing and biodegrading at least one target component, the method including determining a desired level of hydrophobicity of the silica-matrix encapsulated biomaterial, the desired level of hydrophobicity being based on the target component; selecting at least a first and a second silica matrix precursor, wherein one of the first and second silica matrix precursor is more hydrophobic than the other; and forming a silica-matrix encapsulated biomaterial from at least the first and second silica matrix precursors.

Also disclosed is a method of degrading at least one target component, the method including contacting a medium containing the at least one target component and a hydrophobic silica-matrix encapsulated biomaterial, the hydrophobic silica-matrix encapsulated biomaterial comprising a silica matrix and at least one biomaterial, wherein the silica matrix is formed from at least one hydrocarbon moiety containing compound and at least one bridging oxygen moiety containing compound, wherein the target component is degraded by the biomaterial in the hydrophobic silica-matrix encapsulated biomaterial at a rate that is higher than the target component would be degraded by the biomaterial in a silica-matrix encapsulated biomaterial formed without the at least one hydrocarbon moiety containing compound.

A silica-matrix encapsulated biomaterial forming composition including at least one amine group containing silica precursor; and at least one biomaterial. Silica-matrix encapsulated biomaterials formed from such compositions are also disclosed. As well as silica-matrix encapsulated biomaterials formed from such compositions that increase degradation of a target component compared with degradation of the target component by a silica-matrix encapsulated biomaterial formed without the amine group containing silica precursor.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIGS. 1a and 1b show cross sections of illustrative examples of disclosed articles.

FIGS. 2a and 2b show the contact angle versus R (MTMS/Total alkoxide) (FIG. 2a), and images of dispensed droplets on the gel surface for gels formed with various R values (FIG. 2b).

FIGS. 3a, 3b, 3c, and 3d show the florescence intensity of silica gels from a plate reader (FIG. 3a), and fluorescent micrographs of hydrophobic silica gels labeled with Nile red, (λₑₓ=561 nm, λₑₘ=600-700 nm) (fluorescence intensity enhanced for viewing and is not necessarily representative of actual fluorescence) for R equal 0.6 (FIG. 3b), 0.8 (FIG. 3c), and 1.0 (FIG. 3d).

FIGS. 4a and 4b are scanning electron micrographs of silica gels (gels made with a precursor molar ratio (R) above 0.6 formed aggregates of microparticles, whereas all other gels were mesoporous and formed homogeneous structures) at low magnification (3k), scale bar is the same for all images (10 μm) (FIG. 4a) and at high magnification (40k), scale bar is the same for all images (1 μm) (FIG. 4b).

FIGS. 5a, 5b, 5c, and 5d show mechanical properties of the silica gels determined by axial compression testing vs precursor molar ratio, R: Fracture Stress (FIG. 5a), Young's Modulus (FIG. 5b), Strain at Failure (FIG. 5c), and Toughness (FIG. 5d).

FIG. 6 shows the adsorption coefficient of fluorene as a function of precursor molar ratio, R.

FIGS. 7a, 7b, 7c, 7d, 7e, 7f, and 7g show the degradation and adsorption of naphthalene (FIG. 7a), phenol (FIG. 7b), p-Cresol (FIG. 7c), indole (FIG. 7d), p-methoxyphenylmethyl sulfide (FIG. 7e), azulene (FIG. 7f), and phenathrene (FIG. 7g) from a hydrocarbon solution after 48 hours of incubation.

FIG. 8 shows the total substrate removed from the hydrocarbon solution by free and encapsulated cells after 48 hours.

FIG. 9a to 9c show SEM images of gel microstructure—scale bars are the same (FIG. 9a); contact angle measurement of surface hydrophobicity (FIG. 9b); and confocal fluorescence images of silica gels stained with Nile Red—scale bars are the same. Images have been brightened using the LUT to better illustrate the distribution of the hydrophobic methyl groups (FIG. 9c).

FIGS. 10a to 10d show mechanical properties of the silica gels determined by axial compression testing vs MTMS content—Stress at failure (o) (FIG. 10a); Elastic Modulus (E) (FIG. 10b); Strain at Failure (E) (FIG. 10c); and Toughness (FIG. 10d).

FIG. 11a to 11f show adsorption characterization of 4-nitroanisole (FIGS. 11a to 11c) and 4-nitrophenol (FIGS. 11d to 11f). Adsorption kinetics of each chemical measured by UV-Vis over 24 hours (FIGS. 11a and 11d). Relative amounts of each chemical adsorbed to the silica gel (FIG. 11b and lie). Time constant (c) of a model function fitted to the experimental data with the form y=a+be^{-ct} (FIGS. 11c and 11f).

FIGS. 12a, 12b, 12c, and 12d show fluorescence intensities of the four gels doped with Nile red probe at an excitation wavelength of 561 nm (FIG. 12a); SEM image of TePs/TeOs (1:1) gel (FIG. 12b); Confocal image of TePs/TeOs (1:1) gel doped with Nile red probe and histogram of diameter distribution of hydrophobic patches (FIG. 12c); and Z-slice volume confocal image of TePs/TeOs (1:1) gel doped with Nile red probe (FIG. 12d).

FIG. 13 shows atrazine adsorption isotherms (10-100 μM) to silica-gel with four different cross-linkers (TeOs alone, TeOs/TeMs, TeOs/TeVs, TeOs/TePs).

FIG. 14 shows particle distribution of TePs/TeOs and TeOs in SEM image (two diameter peaks 40-60 nm and 80-110 nm).

FIGS. 15a and 15b show adsorption isotherms of three triazines (hydroxyatrazine, atrazine and ametryn) to TeOs (FIG. 15a) and TeOs/TePs (FIG. 15b) gels.

FIGS. 16a and 16b show removal and degradation of 10 μM atrazine (8*20 min washes) by TeOs and TeOs/TePs (1:1) gels—atrazine removal rate for each 20 min wash (FIG. 16a) and hydroxylatrazine formation rate for each 20 min wash (FIG. 16b).

FIGS. 17a, 17b, and 17c show confocal images of TePs/TeOS (1:1) (FIG. 17a), TePs/TeOs (3:1) (FIG. 17b) and TePs (100%) (FIG. 17c) with histogram of hydrophobic patches size

FIG. 18 shows degradation of 10 μM atrazine (6*20 min washes) by TePs/TeOS (1:1), TePS/TeOs (3:1) and TePs (100%) gels, along with confocal images of E. coli expressing GFP encapsulated in TePs/TeOS (1:1) and TePs (100%) gels.

FIGS. 19a, 19b, 19c, and 19d show adsorption and diffusion of atrazine to TeOs (FIG. 19a), 1:1 TePs/TeOs (FIG. 19b), 3:1 TeOs/TePs (FIG. 19c) and TePs (FIG. 19d) as a function of time.

FIGS. 20a, 20b, 20c, and 20d show atrazine removal by the bio-reactive gels and by free cells+cross-linker (FIG. 20a); hydroxyatrazine formation (activity assay) in the bio-reactive gels and by free cells+cross-linker (FIG. 20b); and Confocal images of E. coli expressing GFP mixed with TeOs cross linker solution (FIG. 20c) or TePs/TeOS (3:1) cross linker solution (FIG. 20d).

FIG. 21 shows atrazine adsorption isotherms (10-100 μM) to silica-gel with four different alkoxides ((▪) Teos alone, (▴) Teos-methyl, (▪) Teos-vinyl, () Teos-phenyl).

FIGS. 22a to 22d show Nile red probe λₘₐₓ fluorescent intensity measurement in 10-100% phenyl (FIG. 22a). Confocal image of 75% phenyl-silica gel containing Nile red as a hydrophobic probe (scale bar 100 μm) (FIG. 22b). SEM images of 50% phenyl-silica gel (scale bar 10 μm) (FIG. 22c) and 75% phenyl-silica gel (scale bar 500 μm) (FIG. 22d) showing the large spherical aggregates.

FIGS. 23a to 23d show adsorption of atrazine (100 μM) to silica gels as a function of phenyl alkoxide (FIG. 23a). Adsorption isotherm of three s-triazines (hydroxyatrazine (0-30 μM), atrazine (0-60 μM) and ametryn (0-60 μM)) to Teos (FIG. 23b) and 50% phenyl gels (FIG. 23c). Freundlich binding coefficients of the s-triazine adsorption isotherms (FIG. 23d).

FIGS. 24a and 24b show adsorption of atrazine by (□) Teos, (▴) 50% phenyl gel and (▪) 75% Phenyl gel as a function of time as wet silica-gel (FIG. 24a) and dried granulated silica gels (FIG. 24b).

FIG. 25 shows hydroxyatrazine formation (activity assay) in the bio-reactive phenyl-silica gels as a function of phenyl alkoxide content.

FIGS. 26a to 26d show SEM images of 25% phenyl-silica gel (FIG. 26a), 50% phenyl-silica gel (FIG. 26b) and 75% phenyl-silica gel with encapsulated E. coli (FIG. 26c). Confocal image of 75% phenyl alkoxide aggregates doped with Nile red and E. coli expressing GFP (100 μm scale bar) (FIG. 26d).

FIGS. 27a and 27b show removal and degradation of 10 μM atrazine (6×20 min washes) by () TeOs and (∘) 75% phenyl gel. Specifically, atrazine removal rate for each 20 min wash (FIG. 27a) and hydroxylatrazine formation rate for each 20 min wash (FIG. 27b).

FIG. 28 shows a SEM image of the enhanced adsorption of the biomaterial to hydrophobic microspheres and schematically depicts the interface.

FIG. 29 shows absorbance versus time (min) because of p-nitrophenol, which shows the degradation of parathion over time in a 75% phenyl (25% TEOS) containing silica gel and a 100% TEOS silica gel.

FIGS. 30a and 30b show cross-sectional views (Top view on top left, side views on bottom and right) of multi-layered silica gel—top view of hydrophilic layer with GFP expressing bacteria (FIG. 30a); and top view of hydrophobic layer with Nile red stain (FIG. 30b).

FIG. 31 is a schematic depiction of the diffusional barriers to degradation of a target component by disclosed silica gel matrices.

FIGS. 32a, 32b, 32c, 32d, 32e, and 32f show glutothione transferase (FIGS. 32a and 32b), homoprotocatechuate 2,3-dioxygenase (FIGS. 32c and 32d), and Azo reductase (FIGS. 32e and 32f) enzymatic reactions of whole cells encapsulated in TeOs based gels and free in solution.

FIGS. 33a to 33d show the degradation rate in the various gels for dioxygenase (FIG. 33a), AtzA (FIG. 33 b), cyanuric acid hydrolase (FIG. 33c), and Azo Reductase (FIG. 33d).

FIG. 34a shows the activity of whole cells expressing azo-reductase encapsulated in the silica-APTES matrix over time (Methyl red sodium salt degradation) in the presence of externally added NADPH; and FIG. 34b shows confocal images of DH5α stained with PI in TeOs and APTES gels.

FIG. 35 shows four activity assays of whole cells expressing homoprotocatechuate 2,3-dioxygenase encapsulated in TeOs, TeOs+APTES and APTES gels.

FIG. 36 shows the composition of various gels, the contact angle of a water droplet on the gel and an image of the water droplet on the gel.

FIGS. 37a and 37b show a comparison of in vivo activities of different cyanuric acid hydrolases expressed in E. coli: whole cells in suspension (free cells) (FIG. 37a) and whole cells encapsulated in silica gels (FIG. 37b). E. coli cells were encapsulated as 2-ml cylindrical blocks (3.5-mm thickness and 570-mm2 surface area). Activities are reported as the mean values and standard deviations from triplicate determinations.

FIGS. 38a and 38b show the effect of heat treatment on E. coli cells in suspension and encapsulated in silica blocks. (FIG. 38a) Cells in suspension at different temperatures were exposed to fluorescent dyes testing for cell nonviability/permeability as indicated. (FIG. 38b) Viability of encapsulated cells (200 μl cylindrical blocks with thickness of 6 mm and surface area of 30 mm²) at different temperatures as determined by total loss of metabolic activity shown by oxygen consumption. Data are re-ported as the mean values and standard deviations from triplicate determinations.

FIGS. 39a and 39b show the effect of heat treatment on cyanuric acid hydrolase activity with non-encapsulated cells (FIG. 39a) and encapsulated cells (FIG. 39b). E. coli cells were encapsulated as 2-ml cylindrical blocks. Data were normalized by setting the activity prior to heat treatment as 100%. Activities were measured in triplicate, and the mean values and standard deviations are represented.

FIGS. 40a to 40d show cyanuric acid hydrolase activity in E. coli cells encapsulated in 2-ml cylindrical blocks that had been subjected to different heat treatments and then stored at room temperature for up to 2 weeks. (FIG. 40a) No heat treatment prior to storage. (FIGS. 40b, 40c and 40d) Each enzyme as indicated (TrzD, AtzD, and CAH) was heat treated as described in the examples and then stored at room temperature. Data are normalized with respect to the initial activity at time zero for each set of encapsulated cells. Assays were conducted in triplicate at the indicated times, and the mean values and standard deviations are represented.

FIGS. 41a and 41b show cyanuric acid degradation by E. coli cells expressing CAH enzyme encapsulated in hemispherical silica beads (1.0 to 1.5 mm in diameter) contained within a glass cylindrical column of bead dimensions 2.0 cm (diameter) and 3.0 cm (height) operating in a flowthrough, recirculating mode. (FIG. 41a) Schematic diagram of the system. Channel A, empty reactor; channel B, beads with no cells; channels C and D, beads with encapsulated cells. Controls showed no degradation. (FIG. 41b) Determined concentrations of cyanuric acid in the reservoir with beads containing encapsulated cells as a function of time. The second round was the same bioreactor with the same cells and beads tested with a fresh cyanuric acid solution 1 week later. The inset graph shows the same data for the second round plotted as the logarithm of cyanuric concentration versus time. All data shown are the averages and standard deviations of triplicate determinations.

FIG. 42 shows cyanuric acid degradation by E. coli cells expressing CAH and encapsulated in 1-mm spherical silica beads tested with swimming pool waters from three different sites containing different levels of hypochlorite. The hypochlorite, pH, and cyanuric acid levels were determined as described in Materials and Methods. Since the pools had only recently been opened, cyanuric acid levels were relatively low, and so they were spiked to a level of 100 ppm, at which pools need to be treated. The hypochlorite levels were as follows: control water from lab with 0.0 ppm (♦), pool containing 0.9 ppm (∘), pool containing 1.8 ppm (▪), and pool containing 4.5 ppm (□). For clarity, the numbers denoting the hypochlorite concentration in ppm are adjacent to the respective lines generated from waters with those values. The data were obtained from triplicate samples run in, parallel and the mean values and standard deviations are plotted. The inset graph shows the same data plotted as the initial rate of cyanuric acid degradation versus chlorine concentration. The rate curves show a fit of r²=0.99, except for the 4.5-ppm water, where r²=0.98. The last points at or near the baseline were not plotted, as they are not initial rate points because the cyanuric acid had been depleted.

FIG. 43 shows SEM images of the four types of gels: TeOs, TeMs/TeOs (1:1), TeVs/TeOs (1:1) and TePs/TeOs (1:1). **The measurement bar is of 500 nm.

The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

Disclosed herein are compositions and methods that include a silica containing matrix (or a silica matrix) and a biomaterial. A composition containing a silica containing matrix and a biomaterial encapsulated therein can also be referred to as a “silica-matrix encapsulated biomaterial”. The compositions can be useful in numerous applications where bioremediation or biodegradation of a target chemical or chemicals is desired. Disclosed compositions can enable new and useful application of biomaterials in biotechnology (e.g. biosensing, biocatalysis, bioremediation, and bioreactors) and medicine (e.g. regenerative medicine, tissue engineering, and recombinant protein production), and in new hybrid materials with improved functional and structural properties. The compositions can contain a hydrophobically modified silica matrix with a biomaterial, and such compositions can be referred to as “hydrophobically modified silica-matrix encapsulated biomaterial” or “hydrophobic silica-matrix encapsulated biomaterial”. The hydrophobically modified silica matrix can serve to increase transport of a target component, e.g., an organic molecule, from the media it is in, to the biomaterial while surprisingly not diminishing access of the biomaterial to the target component.

In some embodiments, compositions can include a silica containing matrix formed from at least one compound referred to herein as a non-reactive hydrocarbon moiety containing compound, or simply hydrocarbon moiety containing compound. In some embodiments, silica containing matrices can be formed from at least one hydrocarbon moiety containing compound and at least one bridging oxygen containing moiety. The two components can also be referred to herein as “hydrocarbon moiety compound” and “bridging oxygen moiety compound”. Illustrative bridging oxygen moiety compounds can include alkoxides for example. Illustrative hydrocarbon moiety compounds can include a silicon containing compound having a carbon containing moiety that is not an alkoxide. For example, hydrocarbon moiety compounds can include alkyls, aryls (such as phenyls for example), and vinyls. A silica containing compound that includes at least one substituent that is not a bridging oxygen moiety, e.g., an alkoxide, is considered a hydrocarbon moiety compound herein. Inclusion of a hydrocarbon moiety compound serves to increase the hydrophobicity of a silica containing matrix formed using the moiety.

Examples of bridging oxygen moiety containing compounds can include tetramethyl orthosilicate (which can also be called tetramethoxysilane or TMOS), tetraethyl orthosilicate (which can also be called tetraethoxysilane or TEOS), tetrakis(2-hydroxytehyl) orthosilicate, methydiethyloxysilane, tetrakis(2-hydroxyethyl)orthosilicate (THEOS), 3-(glycidoxypropyl)triethoxysilane (GPMS), 3-(trimethoxy silyl)propylacrylate (TMSPA), N-(3-triethyoxysilylpropyl)pyrrole (TESPP), vinyltriethoxysilane (VTES), methacryloxypropyltriethoxysilane (TESPM), silica nanoparticles (e.g. Ludox or Nyacol), sodium silicate, diglycerylsilane, 3-aminopropyltriethoxysilane (APTS), 3-(2,4-dinitrophenylamino)propyltriethoxysilane, mercaptopropyltriethoxysilane (TEPMS), 3-(2-aminoethylamino)propyltriethoxysilane, and triethoxysilyl-terminated poly(oxypropylene). More than one bridging oxygen moiety containing compound can be utilized to form a silica matrix.

Examples of compounds having at least one hydrocarbon moiety can include silica precursors with moieties chosen from alkyls, and aryls for example. More specific examples of compounds having at least one hydrocarbon moiety can include silica precursors with moieties chosen from ethyl, methyl, propyl, butyl, pentyl, hexyl, phenyl, napthyl, nitrophenyl, anthracenyl, aminophenyl, isoprenyl, furanyl, and n-decyltrimethoxysilane for example. Specific examples of compounds that can be utilized as hydrocarbon moiety containing compounds can include, for example methyltrimethyoxysilane (MTMS), triethoxy-methylsilane (TeMs), triethoxy-vinylsilane (TeVs), and triethoxy-phenylsilane (TePs). More than one hydrocarbon moiety containing compound can be utilized to form a silica matrix.

Silica gel matrices can also be formulated by including one (or more) precursors that have a functional group other than a hydrocarbon enhancing or bridging oxygen moiety. In some embodiments, such compounds can be referred to as functional group containing compounds. Illustrative functional groups can contain, for example amine groups. Illustrative examples of amine group containing precursors or compounds can include, for example 3-aminopropyltriethoxysilane (APTS), and 3-(2-aminoethylamino)propyltriethoxysilane.

Disclosed silica gel matrices can include silicon-oxygen-silicon bonds (which can be described as forming a backbone of the gel matrix) and silicon-carbon bonds. The silicon-carbon bonds form portions that are more hydrophobic than the silicon-oxygen bonds, thereby making the overall matrix more hydrophobic than a matrix without the silicon-carbon bonded portions. Such matrices can be formed using a combination of bridging oxygen moiety containing compounds and hydrocarbon moiety containing compounds. The bridging oxygen moiety containing components form reactive silicon compounds via a hydrolysis route or an alkali metal silicate route and then participate in condensation reactions to form siloxanes (silicon-oxygen-silicon). In some embodiments, silica nanoparticles can also be added. The addition of silica nanoparticles can increase the stiffness of the silica matrix. The silicon-oxygen-silicon bonds form an interconnected network having pores.

Disclosed silica matrices include hydrocarbon moiety containing compounds to increase the hydrophobicity of the overall matrix. The hydrocarbon moiety containing compounds include at least one group (bonded to a silicon) that is not capable of forming a silicon-oxygen-silicon bond. These hydrophobic groups are dispersed in the silica matrix and thereby increase the overall hydrophobicity of the matrix.

Disclosed silica matrices also include a biomaterial. The biomaterial can be described as being encapsulated in the matrix. A biomaterial is any material that has some catalytic activity. The term “biomaterial” refers to one or more microorganisms, cells, or enzymes such as enzymes within a cell or microorganism or enzymes not within a cell or microorganism (free enzymes). In some embodiments, the term “biomaterial” does not include mammalian cells. Examples of biomaterials can include enzymes, macromolecules, and non-mammalian cells, such as for example bacteria, archaea, protists, or fungi. Disclosed compositions can include virtually any type or types of biomaterial. A biomaterial may or may not have a lesser activity when encapsulated in a matrix than it did when free of the matrix.

Disclosed matrices can be described by various properties. For example, a matrix can be described by its porosity, the average (or some other numerical descriptor) pore size, the average agglomerate size, the heterogeneity of the matrix, the surface energy of the matrix, the mechanical properties of the matrix, its chemical composition, a description of the compounds that formed it, or some combination thereof. One matrix is different from a second matrix if at least one of these properties is different in the two matrices.

One way of describing a matrix is by the materials or compounds that formed it. In some embodiments, a disclosed matrix can be formed from at least a first component (which can also be referred to as a first silica matrix precursor) containing a silicon bonded to four alkoxides and a second component (which can also be referred to as a second silica matrix precursor) containing a silicon bonded to less than four alkoxides. The amounts of these two components can be described by molar ratios. For example, the amounts of the two could be described by the molar ratio of the first component to the molar ratio of the second component.

From the components the amount of at least two can be chosen in order to effect the hydrophobicity (measured by contact angle for example) of the matrix, the porosity of the matrix, the average pore size of the matrix, the average agglomerate size, the permeability, the surface charge, the surface functionality, the fracture stress (σ_{f}) of the matrix, the Young's (elastic) modulus (E), the strain at failure (ε_{f}), toughness (Uₜ), or any combination thereof, for example. In some embodiments, the amounts of the at least two components can be chosen based on a desired level of hydrophobicity of the silica matrix. Such a desired level of hydrophobicity can be based, at least in part, on a target component. For example, the hydrophobicity of the matrix can be selected, based on the amounts of at least the first and second component, to increase the transport of the target component from a medium (a medium can refer to any system in which the target component is contained, specific examples can include, for example water, gas, or combinations thereof) into the matrix. In some embodiments where a target component is an organic compound, a more hydrophobic matrix may show an enhanced transport from the medium to the matrix. In some embodiments, properties other than the hydrophobicity of the matrix can also be considered. For example, increased amounts of hydrocarbon moiety containing compounds can decrease desirable mechanical properties of the matrix, therefore desirable target component transport properties of the matrix may, in some instances be balanced against undesirable decreases in mechanical properties.

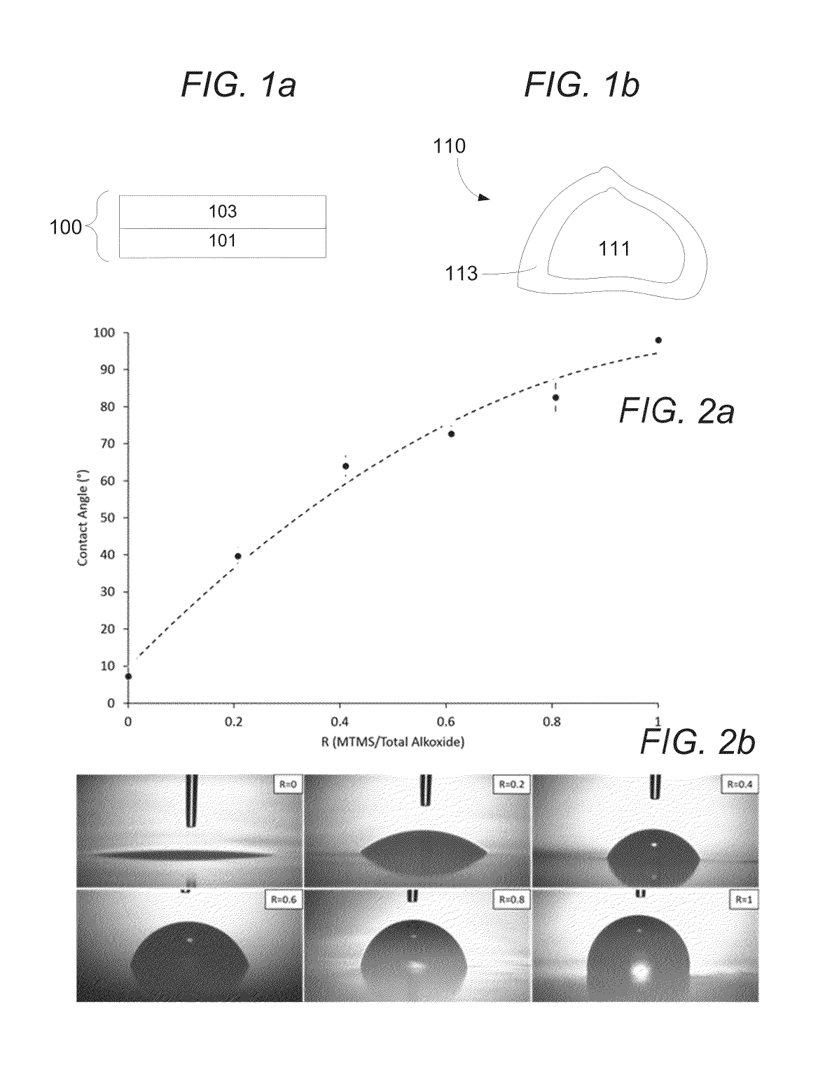

Also disclosed herein are compositions or articles that include at least two silica gel matrices, where the two silica gel matrices are different in at least one way. FIG. 1a shows a cross section of an illustrative example of an article 100 that includes a first silica gel matrix 101 that could be described as a layer and a second silica gel matrix 103 that could be described as a layer. The first layer 101 includes a first silica gel matrix that includes a first silica gel and a first biomaterial. The second layer 103 includes a second silica gel matrix that includes a second silica gel and a second biomaterial. The article 100 could be described as a multilayer article, a laminate, a two-dimensional article, or combinations thereof. In some embodiments, additional layers could also be added to the article 100.

FIG. 1b shows a cross section of another illustrative example of an article 110 that includes a first silica gel matrix 111 and a second silica gel matrix 113. The article 110 can be described as a three dimensional article. In some embodiments, articles such as 100 and 110 can be formed via coating methods (e.g, spin coating, dip coating, etc.), printing (e.g., ink jet printing, bioprinting, etc.), other commonly utilized methods (e.g. gas phase deposition), or combinations thereof.

Also disclosed herein are materials where the first silica gel matrix and the second silica gel matrix exist at different portions of the material. For example, a first portion of a material can predominantly (e.g. not less than 50%) include a first silica gel matrix and a second portion of a material can predominantly include a second silica gel matrix.

The first silica gel matrix and the second silica gel matrix in disclosed articles have at least one property that is different. For example, the biomaterial could be different, the porosity of the first and second silica gel matrix could be different, the average pore size of the first and second silica gel matrix could be different, the surface energy of the first and second silica gel matrix could be different, or any combination thereof. In some embodiments, the surface energy (e.g., hydrophilic or hydrophobic) of the first and second silica gel matrices could be different. For example, in some embodiments, a first silica gel matrix or vice versa could be hydrophilic (e.g., was formed only from bridging oxygen moiety containing compounds, like alkoxides for example) and a second silica gel matrix could be hydrophobic (in comparison to the first silica gel matrix) (e.g., contains bridging oxygen moieties, like alkoxides for example and hydrocarbon moieties, like alkyls, aryls, or vinyls for example).

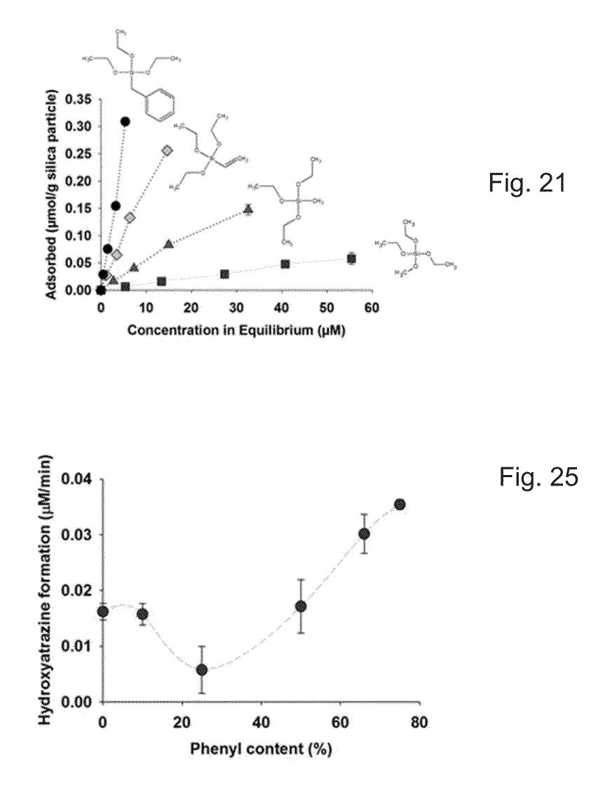

Disclosed silica gel matrices can also include moieties other than bridging oxygen moieties and hydrocarbon moieties. In some embodiments, amine moieties can also be included in the silica gel matrix. Amine moieties can be useful for altering one or more properties of the biomaterial. Silica-gel materials have been used to encapsulate bacteria and enzymes for biocatalytic purposes, yet, degradation rates have been shown to be significantly lower for encapsulated cells in comparison to free cells in solution which limit the effectiveness of their application. The reduction in degradation rates is due to two main diffusional barriers: Low diffusion and adsorption rates to the silica-gel matrix, and low transfer rates through the cell membrane. FIG. 21 depicts the two diffusional barriers to the degradation of atrazine and parathion (illustrative target components) by a biomaterial (biodegrading enzyme in FIG. 21).

In some embodiments, an amine cross linker can be utilized in the silica-gel synthesis. It is thought, but not relied upon that the amine cross linker can break down the membrane diffusion barrier and thereby increase degradation rates of a target component.

Disclosed silica gel matrices can be made using reactive schemes known to those of skill in the art. Illustrative methods of making disclosed silica gel matrices can include combining the silica gel matrix precursors (bridging oxygen containing silica precursor, hydrocarbon moiety containing silica precursor, or combination thereof) and hydrolyzing (e.g., via the addition of acid) the silica gel matrix precursors. The hydrolyzed precursor solution (e.g., after being neutralized) can then be added to silica nanoparticles (if being utilized) and the biomaterial. The amounts of the various components can be based on desired properties to be obtained and the starting materials. Other components and steps can also be added to methods of making

Disclosed silica gel matrices and/or articles including such silica gel matrices can be utilized for various applications. For example, it can be used for the treatment of water, wherein the biomaterial can transform one or more chemicals in the water into other chemicals, such as chemicals that are less toxic. Any suitable biomaterial can be used to treat water. A specific example includes the treatment of atrazine-containing water, to covert at least some of the atrazine therein to a different chemical. Another specific example includes the treatment of water that contains pesticides, herbicides, fungicides, insecticides, or other pollutants, for example pollutants from industrial processes or oil and gas drilling processes.

Another example includes the treatment of fracking water (the term “fracking water” as used herein refers to water used in or produced from a hydraulic fracturing process, for example, fracking water includes any water that is released, or polluted at any time during hydraulic fracturing for oil or gas), wherein the biomaterial can degrade chemicals that can be present in fracking water. In various methods, disclosed gel matrices can provide methods of degrading chemicals in fracking water, for example to decontaminate the water or to make the water less toxic. Hydraulic fracturing is a process used to recover natural gas/oil from deep shale formations. Large amounts of water, sand and additives are pumped under high pressure to create fractures, which allow the gas to travel to the surface for collection. Hydraulic fracturing fluid also contains many materials, including for example acids, biocides, breakers, clay stabilizers, corrosion inhibitors, crosslinkers, defoamers, foamers, friction reducers, gellants, pH control, propants, scale control and surfactants.

Disclosed compositions may be useful for remediation of byproducts of the hydraulic fracturing. Hydraulic fracturing is a nonconventional method for extraction of oil and gas which pollutes extensive amounts of fresh water. This process involves pumping water, sand, and chemicals into deep shale wells at high pressures to create fractures, releasing oil, natural gas, and other organic compounds. Fracking requires 2 to 4 million gallons of fresh water per well for each operation, which can be repeated up to 20 times per well. The water used during these operations, highly polluted with the added chemicals and the hydrocarbons from the well, is then recovered prior to oil and gas extraction (produced water).

Samples of hydraulic fracturing produced water may contain over one thousand organic compounds, various salts, numerous inorganic elements, and metals. Many of the chemicals found in the produced waters are known toxins, mutagens, and carcinogens and pose an enormous hazard to the environment and human health. Polycyclic aromatic hydrocarbons (PAH) are of particular concern due to their persistence and established carcinogenic potential.

Bioremediation is a sustainable and permanent solution for removal of PAH from water and has advantages over conventional treatment technologies. These technologies, including membrane filtration, thermal desalination, and evaporation ponds, do not target specific pollutants nor do they degrade the chemicals, instead they concentrate the PAH for disposal in a landfill. Bioremediation is a process in which microorganisms are used to degrade and effectively destroy target chemicals. Natural microorganisms which can biodegrade PAH are ubiquitous in the environment and can be harnessed for the treatment of produced waters through bioencapsulation, where the cells are confined within a 3D structure.

Bioencapsulation of bacteria has been used extensively for bioremediation of pollutants. In some cases, it has been shown to protect the entrapped cells from predation, some environmental stressors, and toxicity of high concentration pollutants. In addition, bioencapsulation may allow the bacteria to be utilized within industrial flow through treatment devices. Silica hydrogels (gels) are of great interest for this purpose, due to cytocompatible synthesis, tunable microstructure, chemical and biological stability, and mechanical strength. However, the inherent hydrophilic surface characteristics of typical silica gels may limit the diffusion and adsorption of the hydrophobic PAH found in the produced waters, ultimately reducing their removal. Organic modification of silica gels has been shown to improve the diffusivity of a hydrophobic molecule and increase the adsorption of hydrocarbons.

Disclosed compositions may be useful for remediation of water contaminated with agrochemicals. Agrochemicals, such as herbicides, are indispensable, yet their use has led to severe contamination. Atrazine (2-chloro-4-ethylamino-6-isopropylamino-1,3,5-triazine) is a pre-emergent herbicide that is widely used in the United States and an analog, terbuthylazine, is used in the EU. The widespread occurrence of atrazine and related herbicides in the environment has led to it being one of the most studied agro-chemicals (with over 12,000 articles published in the last 70 years) and a continuous effort is being put into developing suitable treatment methods to promote optimum environmental stewardship.

Currently, the commonly applied remediation method for organic agro-pollutants, such as atrazine, is adsorption, primarily by granulated activated carbon (GAC). However, due to diffusional and specificity limitations specific adsorption to advanced materials such as polymeric resins, carbon nanotubes, clay minerals and oxides has also been extensively studied. The main drawback of these materials is that they only concentrate the pollutant on the solid matrix, requiring follow-up steps to dispose of the concentrated waste; for example, by landfilling or incineration. It is preferable from both an economic and an environmental perspective to develop remediation strategies that could simultaneously adsorb and degrade the chemicals in situ and in a continuous fashion.

Biodegradation has therefore attracted attention, with different studies employing wild type and recombinant bacteria; and transgenic plants. Recombinant E. coli has been used to express atrazine degrading enzymes to bioremediate a spill of 1,000 pounds of atrazine. They succeeded in reaching a level of herbicide in the soil that was acceptable by regulatory agencies.

Nevertheless, bioaugmentation using specifically-cultivated microorganisms has had limited application because of the problems associated with storing, transporting and application of the cells in an active form. Encapsulation of the bacteria in solid mesoporous matrices provides physical/mechanical protection and has therefore emerged as a promising method for overcoming some of the technical difficulties. Furthermore previous studies have shown it can be advantageous for enhancing biocatalytic reactions by employing higher than natural concentrations of bacteria and enzymes; protecting bacteria from predation, the environment from accidental release; and increasing long term stabilization. Such hybrid materials can also be fine-tuned to control reaction rates and yields; and have potential for easier handling, recycling, storage and packaging.

The desirable matrix should be cost-effective, non-toxic, scalable, biologically compatible and allow transport of the substrate to the cell or enzyme. Therefore, numerous factors should be considered in the choice of material, such as the chemical composition, surface morphology, and mechanical stability. Silica-based matrices offer many of these desired properties: they have a tunable surface area and porosity; biocompatibility, thermal and mechanical stability and are chemically inert as well as resistant to microbial attack. Furthermore, silica-gel encapsulation methods can be carried out under mild conditions via the sol-gel process, allowing for biological protection during the cell encapsulation stage.

Many encapsulation methods, which focus on fine-tuning the desirable properties of the materials, have been published. Some of these studies have dealt with encapsulation of atrazine degrading bacteria and for example, some achieved high atrazine degradation by encapsulating Pseudomonas sp. ADP in electro-spun hollow polymeric microfibers. The initial degradation rates were lower than free cells, but after a growth period of 3-7 days, degradation significantly increased. In other studies, a silica-gel matrix that contained encapsulated non-viable atrazine-degrading bacteria was developed and tested. The degradation of atrazine to hydroxyatrazine was achieved by encapsulated recombinant E. coli expressing AtzA. The focus of these studies was on enhancing the encapsulating material's physical and mechanical properties in terms of diffusion, pore size, mechanical strength and long term stability. Initially, it was shown that the encapsulated E. coli cells expressing AtzA were able to maintain high, constant degradation activity for up to four months. Later, an improved silica-gel material was developed based on silica nanoparticles that were cross-linked by tetraethyl-orthosilicate (TeOs) alkoxide. The method for encapsulation was optimized allowing greater diffusivity, enzyme activity, and long-term mechanical stability. This study was then further expanded to create a general steady state reaction/diffusion model for the encapsulated AtzA expressing bacteria which optimized the matrix in terms of mechanical properties and material/operational costs while sustaining desirable biodegradation rates.

Examples presented here address removal rate, capacity and efficiency issues. The illustrated material has a dual functionality that combines the advantages of adsorption and biodegradation into a single system. This allows for efficient and continuous removal as well as enhanced degradation.

The limited numbers of studies that have dealt with the concept of dual biodegradation and adsorption mechanisms have shown that one mechanism generally suppresses the other. It has also been observed that in activated carbon, adsorption is the prevalent mechanism during the initial stages of a flow through column system. Once the active biofilm is formed, the adsorption kinetics are significantly hindered and the governing mechanism becomes biodegradation. This was also observed in a kinetic study on biotic and abiotic removal of chlorophenols by activated carbon, where the formation of biofilm on the activated carbon and occurrence of biodegradation was shown to reduce the concentration gradient of chlorophenol, thus retarding the adsorption process and resulting in lower removal rates. In another study GAC was evaluated as an adsorptive carrier of Pseudomonas sp. ADP for the degradation of atrazine and was compared to non-adsorbent carriers such as sintered glass beads. The results revealed that the initial degrading efficiency was comparable, but over time the GAC carrier was more stable and did not lose activity. This was attributed to the advantages of the GAC reactor over the non-adsorbing carrier to an adsorption-desorption mechanism providing a favorable microenvironment for atrazine-degrading bacteria. No evaluation of the adsorption capabilities and the impact on the degradation was made.

### Examples

### 1. Remediation of PAH

The goal of this example was to enhance the removal of PAH without compromising the structural integrity of the porous silica gel matrix. TMOS (tetramethoxysilane) and MTMS (methyltrimethoxysilane) were used to synthesize silica gels with varying hydrophobicities, ranging from hydrophilic to hydrophobic in order to determine the effect of the gel hydrophobicity on encapsulated Pseudomonas putida NCIB 9816-4 bioremediation and gel adsorption. The alkoxide precursor molar ratio of MTMS to the total alkoxide in the gel (R) was varied from 0 to 1 to achieve a range of gel hydrophobicity. As R goes from 0 to 1, the matrix becomes more hydrophobic. The gels were characterized to determine their hydrophobicity, microstructure, mechanical properties, adsorption, and biodegradation activity.

### Materials:

Silicon alkoxides and silica nanoparticles for gel preparation were purchased from Sigma-Aldrich (Sigma-Aldrich Corp., St. Louis, Mo., USA): tetramethoxysilane (TMOS, 98%), methyltrimethoxysilane (MTMS, 98%), and Ludox TM-40 colloidal silica nanoparticles (SNP, 40% w/w). All other chemicals were purchased from Sigma-Aldrich and used without further purification. Ultrapure water (UPW) was prepared by filtering distilled water through a Milli-Q water purification system (Millipore, Billerica, Mass., USA) to a final electrical resistance of >18.2 MΩ/cm.

### Bacterial Strains and Growth Conditions:

Cultures of Pseudomonas sp. NCIB 9816-4 were grown on Luria Broth (LB) at 30° C. for about 8 hours and used to inoculate minimal media (MM) at OD₆₀₀ of 0.01. MM was made according to previous methods (Turner, K., Xu, S., Pasini, P. & Deo, S. Hydroxylated polychlorinated biphenyl detection based on a genetically engineered bioluminescent whole-cell sensing system. Anal. . . . 79, 5740-5745 (2007)), with the following substitutions (Hutner's Metals): 318 mg of Na₂EDTA.2H₂O, 24 mg of CoSO₄.7H₂O, 17.7 mg of Na₂B₄O₇.10H₂O. The MM was supplemented with 1 g naphthalene per 300 mL media. Cultures were grown in 2 L shake flasks (230 rpm) for 18 hours at 25° C. with vigorous aeration. Cultures reached a final OD₆₀₀ of 1.5 to 2.5 and were filtered through glass wool to remove any naphthalene crystals prior to harvest. E. coli DH5α was grown in LB shake flasks at 37° C. Cell cultures were harvested by centrifugation at 5000×g for 10 min. Cells were resuspended at 0.5 g (wet weight)/mL in PBS (phosphate buffered saline) for encapsulation.

### Silica Gel Synthesis:

The following precursor molar ratios (R) of MTMS to total alkoxide were used: 0, 0.2, 0.4, 0.6, 0.8, and 1. The desired amount of silicon alkoxide (TMOS/MTMS) was mixed with UPW and 1M HCl in a volumetric ratio of 1:1:0.005, respectively. This mixture was stirred for 2 hours to hydrolyze the precursors. The hydrolyzed precursor solution was then added to the SNP and PBS or bacteria suspension in a volumetric ratio of 4:1:1, respectively. Bacterial cells were not used in any of the characterization studies except for the biodegradation measurement.

### Hydrophobicity Measurements:

Water contact angle measurements were performed to determine the wettability of the synthesized gels, which is a measure of gel hydrophobicity. An MCA-3 image analysis contact angle meter (Kyowa Science Interface Co., Japan) was used with a 30 μm glass capillary tube and a static pressure of 15-30 kPa for droplet generation with distilled water as the probe liquid. Samples were prepared by pipetting 300 μL of gel into a thin film on the surface of a glass microscopy slide. Reported contact angles were averaged from 10 droplet measurements performed in different locations on the sample.

Nile Red was used as a secondary probe for determining gel hydrophobicity, both qualitative and quantitative. Confocal microscopy was performed with a Nikon A1si spectral confocal system mounted on a Nikon Ti2000E inverted fluorescence microscope with DIC optics (Nikon Instruments Inc., Melville, N.Y., USA). NIS Elements imaging software was used for image acquisition and analysis. Nile red from a stock solution (100 μg/mL in EtOH) was added to the sol before gelation at a final concentration of 6.25 μg/mL. 50 μL of gel was prepared onto glass microscopy slides for imaging. Samples were excited at 561 nm and emission was read from 600-700 nm. Samples were performed in triplicate.

A Molecular Devices SpectraMax M5 plate reader was used to quantitate the fluorescence of the samples (Molecular Devices, LLC., Sunnyvale, Calif., USA). 300 μL of gel samples were prepared in a clear-bottom 96-well plate with black sides. Samples were excited at 561 nm, with a cutoff filter at 590 nm, and emission was read from 600-700 nm. Samples were performed in triplicate.

### Evaluation of Microstructure:

Gel samples were examined with a scanning electron microscope (SEM) (Hitachi S-4700, Cold Field Emission Gun). The samples were gradually dried in increasing ethanol concentrations (50%, 75%, 100%) before critical point drying with carbon dioxide (Tousimis Model 780A). The samples were sputter coated with 50 Å of platinum before examination with SEM.

### Mechanical Testing:

Gels were synthesized for evaluating the mechanical properties by producing cylindrical test samples. The final mixture was poured into cylindrical molds for gelation of the sol. After 24 hours, the samples were removed from the molds and placed into PBS for 3 days to allow the gels to age. The molds produced samples with initial dimensions of 12.5 mm diameter×12.5 mm height, but significant shrinkage occurred during aging. Sample diameter and height were measured immediately before testing. The samples were tested in axial compression on an MTS QT10 mechanical testing machine (MTS Systems, Eden Prairie, Minn.) with a loading rate of 1 mm/min until failure. Reported values were averaged from 10 samples. Calculation of the elastic modulus, strain at failure, and toughness were done in Matlab (Mathworks, Inc., Natick, Mass., USA). Toughness was calculated as the area under the stress-strain curve up to the maximum compressive stress.

### Adsorption & Biodegradation Measurements:

Fluorene was used to measure the adsorption coefficient in the synthesized gels. Equilibrium adsorption experiments were performed by making 1 mL gel slabs in 20 mL scintillation vials, then 5 mL of 10 μM fluorene was added, and finally the vials were covered with Teflon tape before being sealed. After 48 hours, the solution was extracted with 1 mL of methyl tertiary butyl ether (MTBE) and analyzed by GC-MS. Samples were performed in triplicate.

For measurement of the biodegradation and total removal, 1 mL silica gel slabs were formed in the bottom of 125 mL serum bottles. Negative and positive control samples were made, containing 40 mg non-degrading Escherichia coli DH5α or NCIB 9816-4 free cells, respectively. 3 mL of a hydrocarbon solution containing 150 μM each of phenol, p-cresol, indole, p-methoxyphenyl methyl sulfide (p-Mpms), azulene, naphthalene, and 10 μM of phenanthrene in PBS was added to the samples. The vials were crimp sealed with polytetrafluoroethylene backed silicone septa. An initial sample was extracted immediately with 1.5 mL MTBE and subsequent samples were incubated on a rotary shaker at 100 rpm and extracted after 48 hours before being analyzed by GC-MS. Extracted samples were separated with an HP-1ms column (100% dimethylsiloxane capillary; 30 m×250 m×0.25 μm), at a helium flow rate of 1.75 mL/min, and a temperature of 250° C. at the injection port. The samples were split at the column outlet between a flame ionization detector (FID, 7890A, Agilent, Palo Alto, Calif., USA) and a mass spectrometer (MS, 5975C, Agilent). An initial temperature of 60° C. was held for 3 minutes before ramping up to 320° C. at 15° C./minute and holding for 6 minutes. Electron impact mass spectra were collected at 70 eV with positive polarity. Samples were performed in triplicate. The octanol/water partition coefficient (Log P) of each substrate was calculated using ChemBioDraw Ultra 14 (PerkinElmer Informatics, Waltham, Mass., USA). The substrates, Log P, and K_{o/w} can be seen in Table 1 below:

TABLE 1
Structures and octanol/water partition coefficients
(LogP) of the substrates used for
biodegradation measurement of Pseudomonas sp. NCIB 9816-4
encapsulated within the silica gels.

| Substrate | LogP | Structure |
|---|---|---|
| Phenol | 1.64 | 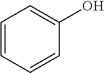 |
| p-Cresol | 1.97 | 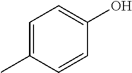 |
| Indole | 2.13 | 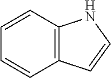 |
| p-methoxyphenyl methyl sulfide | 2.65 | 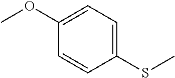 |
| Azulene | 3.32 | 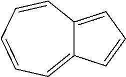 |
| Naphthalene | 3.32 | 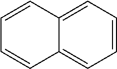 |
| Phenanthrene | 4.49 | 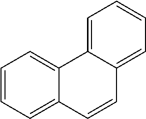 [SMILES: c1ccc2c(c1)ccc1ccccc12] |

### Results

### Surface Hydrophobicity Characterization:

FIG. 2a illustrates the contact angle between water droplets and the gel surfaces. The contact angle increased with increasing precursor ratio (R). The highest contact angle (98.0±1.5°) was achieved with R=1, while the lowest angle (7.3±1.0°) was achieved with R=0. FIG. 2b shows images of dispensed water droplets on the surfaces of gels having the noted R values.

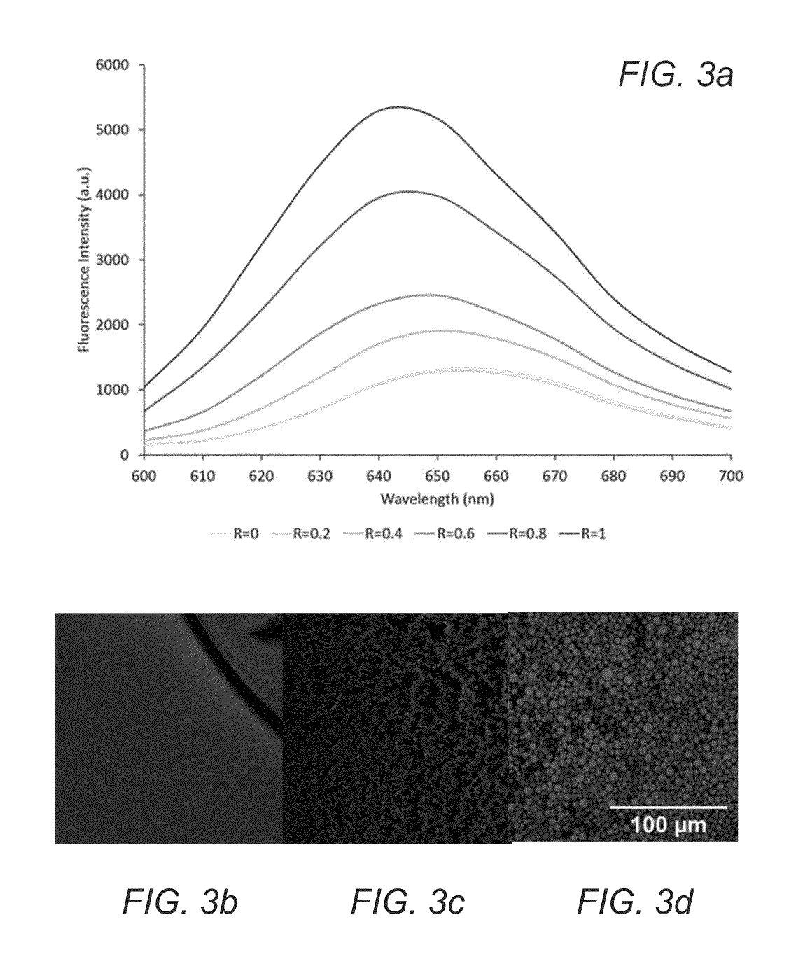

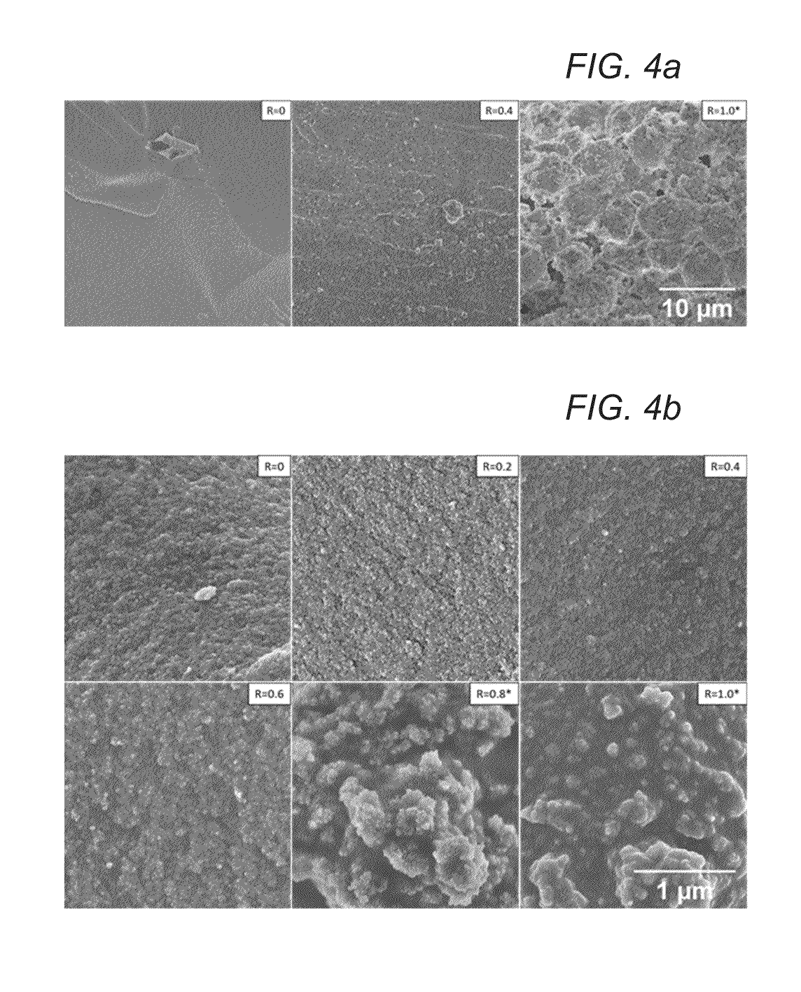

Nile red was used as a fluorescent probe to identify hydrophobic regions within the gels and as a secondary measure of gel hydrophobicity. With R<0.6, there was almost no observable fluorescence under the confocal microscope (data not shown). When R=0.6, the gel had low fluorescence, but it was uniform across the sample (FIG. 3b). For R=0.8, there was a drastic shift in the fluorescence pattern (FIG. 3c). There appeared to be small microparticles dispersed in the sample. For R=1.0, the particles were significantly larger in size, approximately 1-10 μm in diameter (FIG. 3d). These particles were consistent with the size of the particles observed with the same gel under SEM (FIG. 4a). The fluorescence of nile red was quantified by synthesizing gels in a 96 well plate and recording the emission spectra. The fluorescence intensity increased with increasing R, which was consistent with the contact angle measurements, as seen in FIG. 3a. The peak fluorescence intensity ranged from 1310.18±51.41 (R=0) to 5295.60±113.86 (R=1). Additionally, a peak shift was observed, with λₘₐₓ decreasing with increasing R, from 650 nm (R=0) to 640 (R=1). The increase in fluorescence intensity and peak shift to lower wavelengths for more hydrophobic gels was expected and consistent with studies in the literature.

### Microstructural Characterization:

The microstructure of the synthesized gels were studied via SEM. At lower magnification, the formation of large microparticle agglomerates (diameter>1 μm) was observed at R=0.8 and 1 (FIG. 4a). Additionally, a drastic increase structural heterogeneity was observed for those gels. The micrographs showed that the gels were mesoporous (˜5 nm pores), with the exception of gels R=0.8 and R=1.0 (FIG. 4b).

### Mechanical Properties:

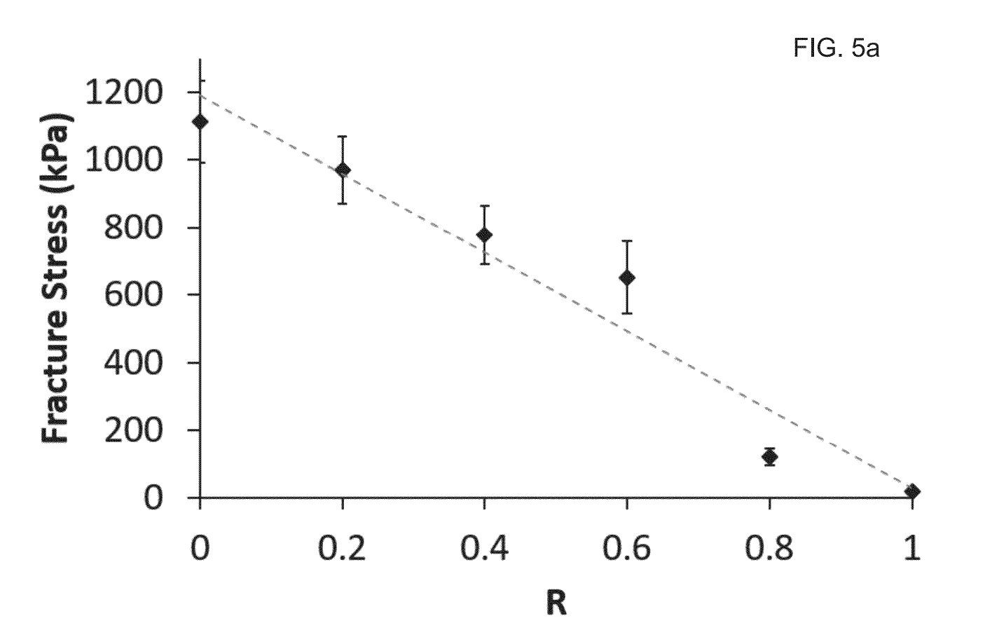

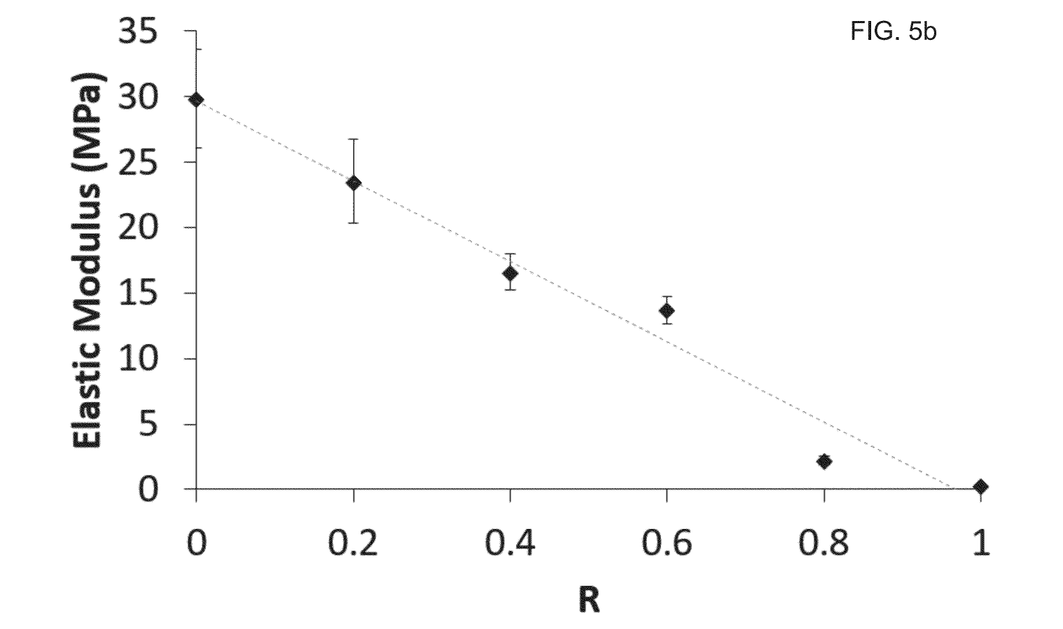

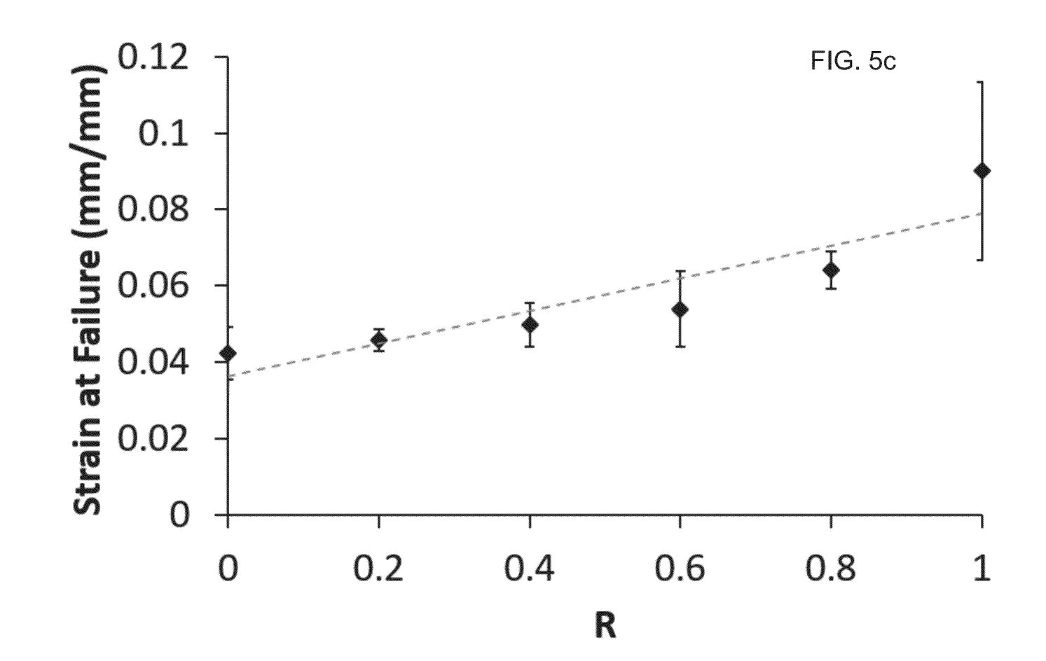

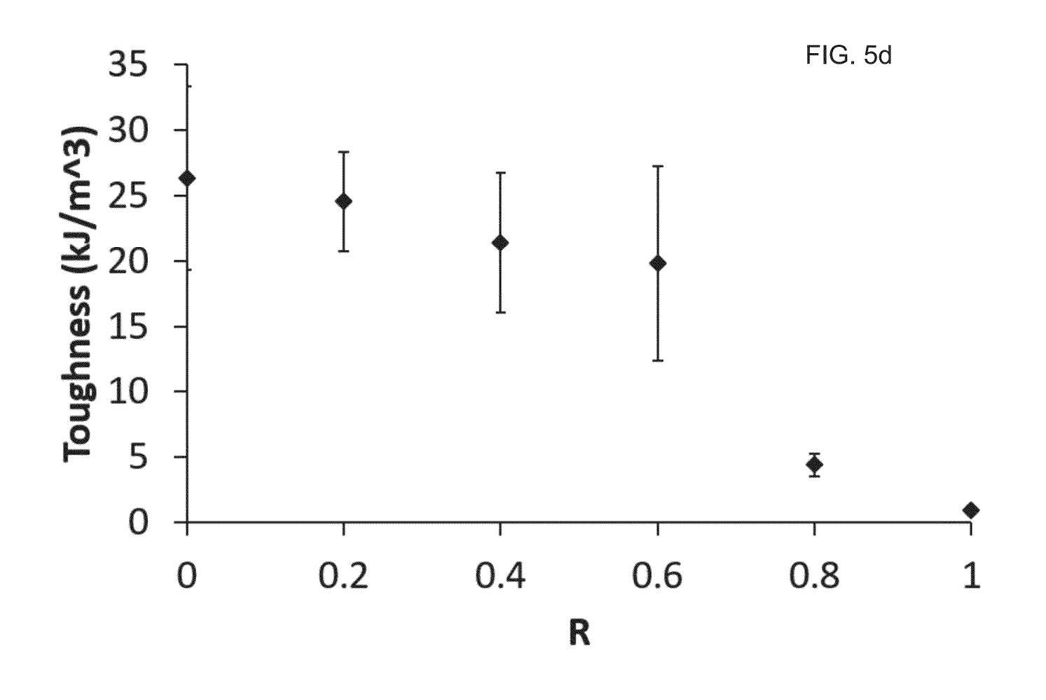

The mechanical properties of the silica gels were evaluated based on fracture stress (σ_{f}), Young's (elastic) modulus (E), strain at failure (ε_{f}), and toughness (Uₜ), (FIG. 2). The fracture stress and elastic modulus decreased with increasing precursor molar ratio (R), with maximum values of σ_{f}=1.1±0.1 MPa, E=29.9±3.8 MPa (R=0) and minima of 16.4±3.4 kPa, 276.8±40.9 kPa (R=1), respectively. The strain at failure increased with R, ranging from 0.042±0.007 (R=0) to 0.09±0.023 (R=1). The toughness decreased only slightly up to R=0.6, from 26.3±7.0 kJ/m³ to 19.8±7.4 kJ/m³, after which it dropped drastically to 4.4±0.4 kJ/m³. Due to their extremely low mechanical properties, gels with R=0.8 and 1 were excluded from all subsequent biodegradation studies. FIGS. 5a, 5b, 5c, and 5d show mechanical properties of the silica gels determined by axial compression testing vs precursor molar ratio, R: Fracture Stress (FIG. 5a), Young's Modulus (FIG. 5b), Strain at Failure (FIG. 5c), and Toughness (FIG. 5d).

Adsorption/biodegradation of hydrocarbons: The equilibrium adsorption experiments with fluorene showed that the adsorption coefficient of fluorene (K_{d}) increased more than two orders of magnitude from the most hydrophilic gel, 4.43±2.62 mL/g (R=0), to the most adsorptive gel, 681.88±26.21 mL/g (R=0.8). For R=1, K_{d} dropped to 435.85±52.25 mL/g. FIG. 5 shows the adsorption coefficient of fluorene as a function of precursor molar ratio, R.

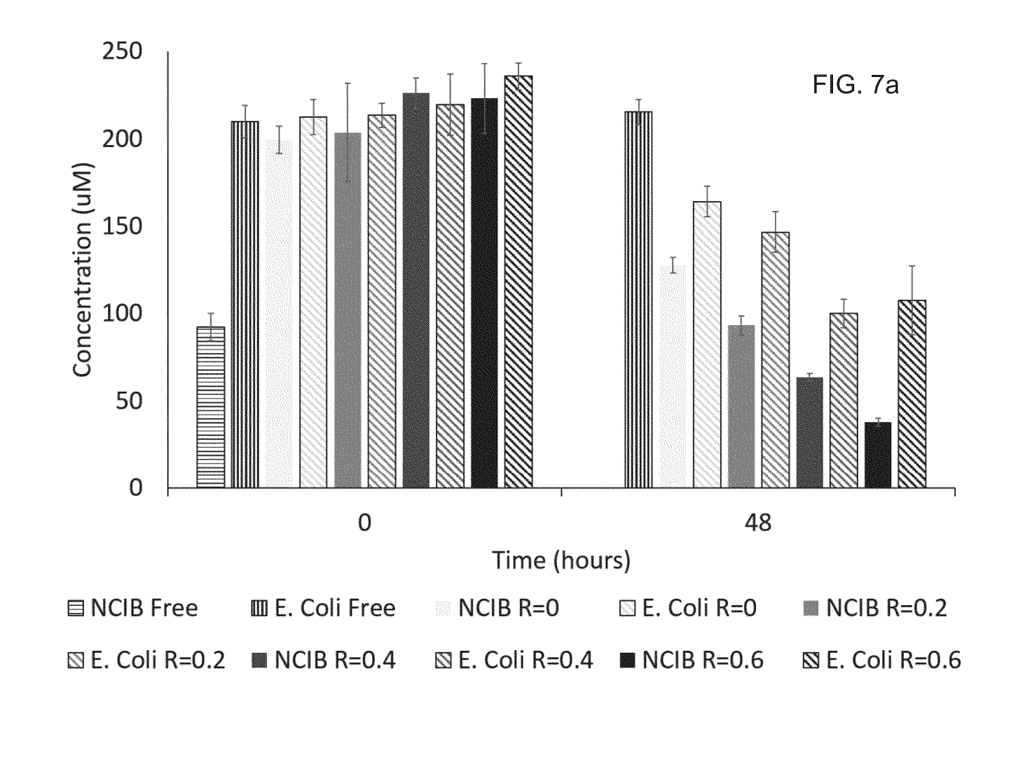

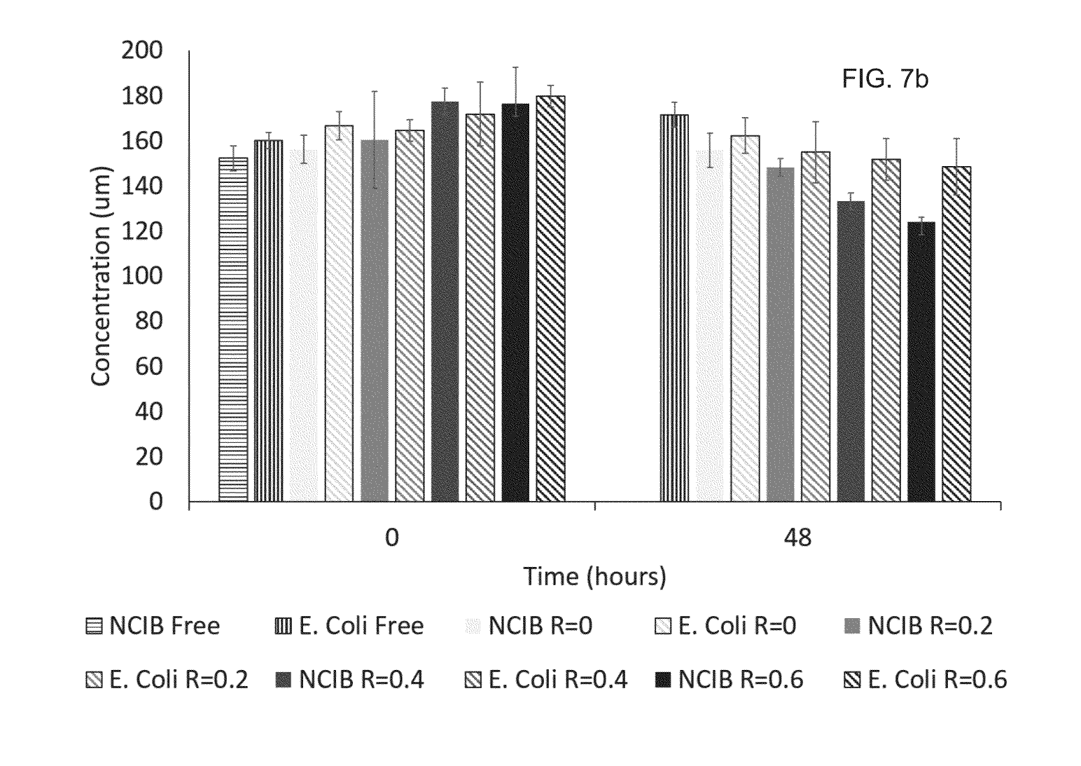

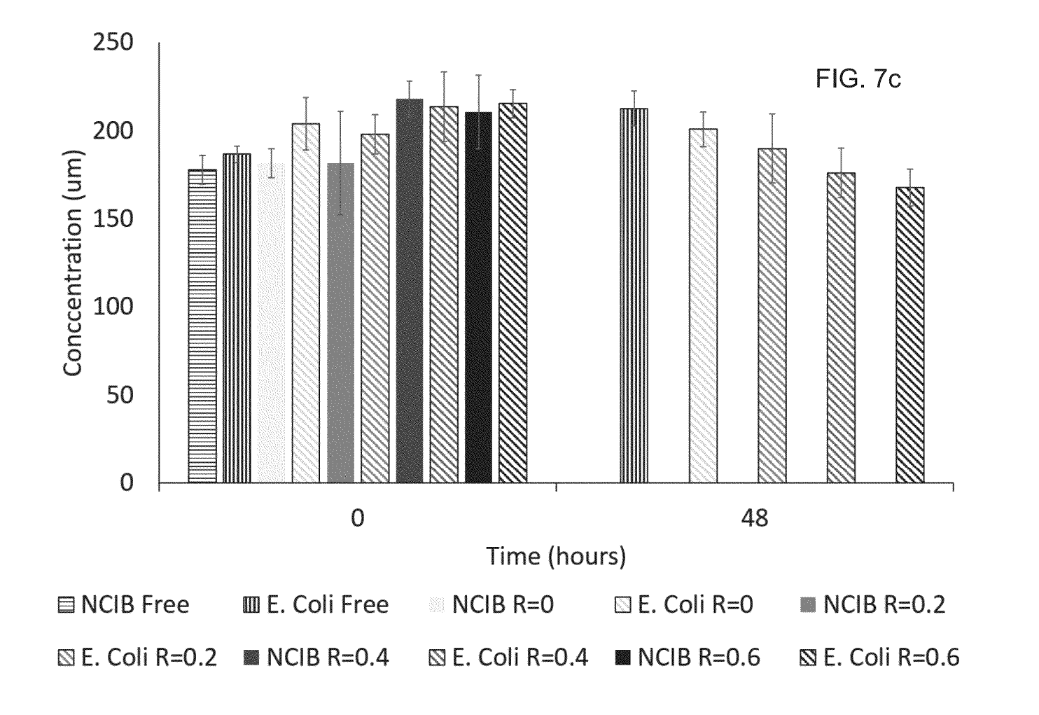

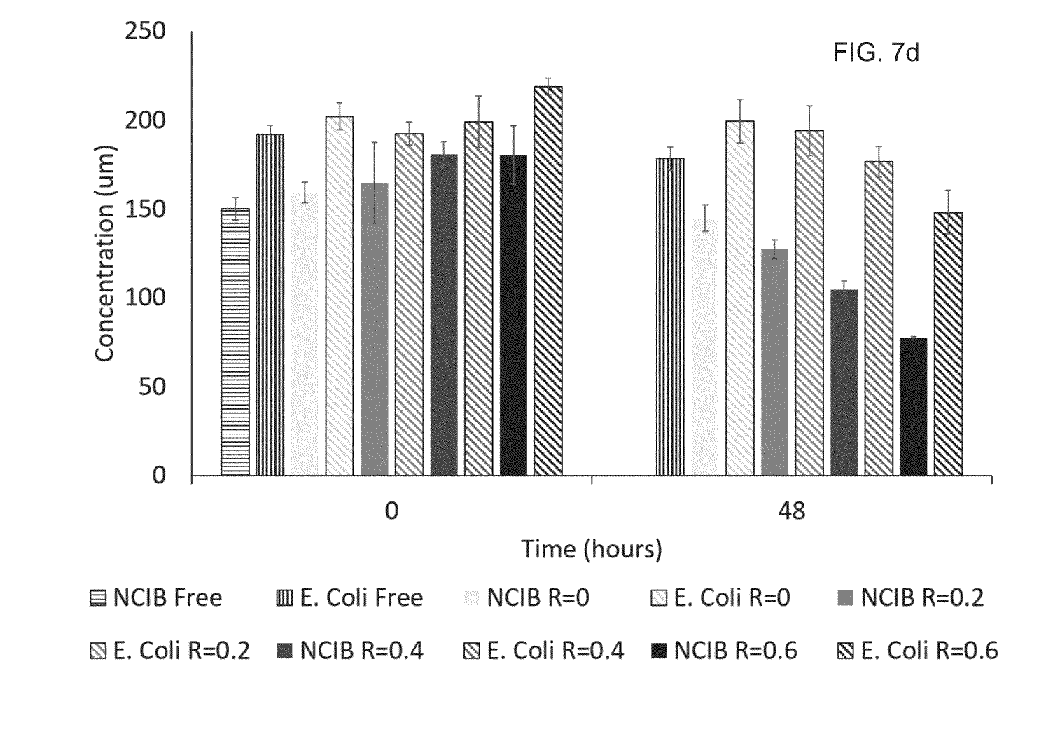

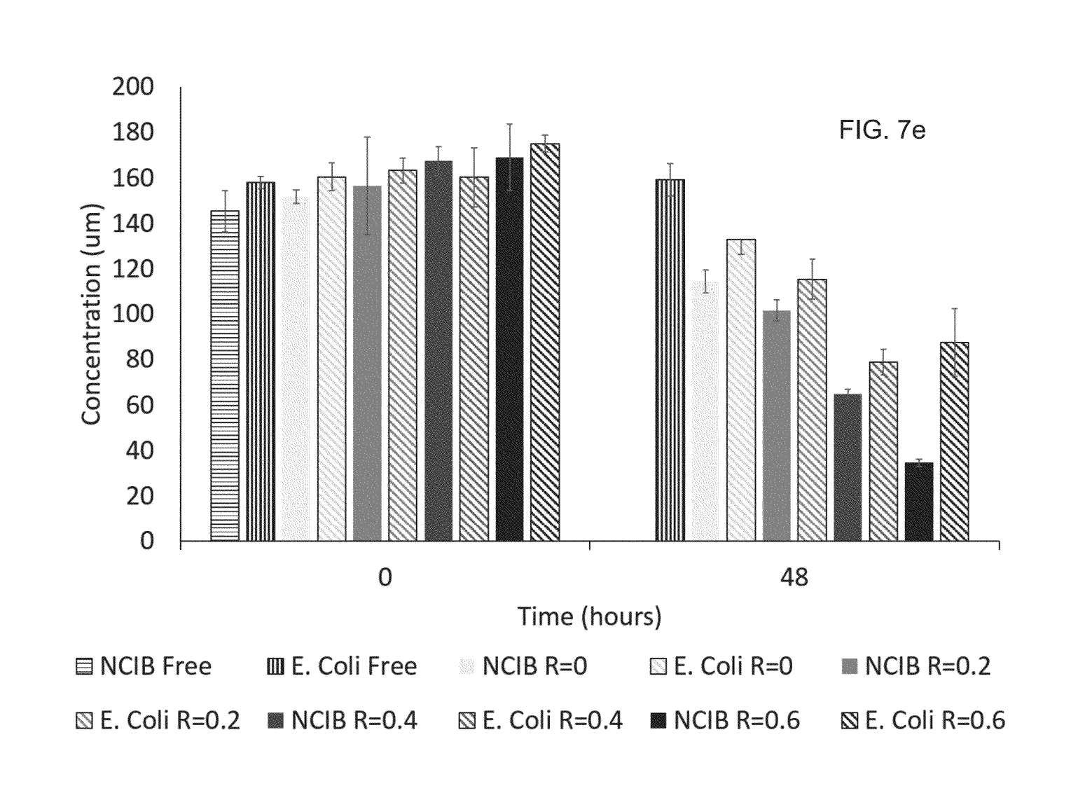

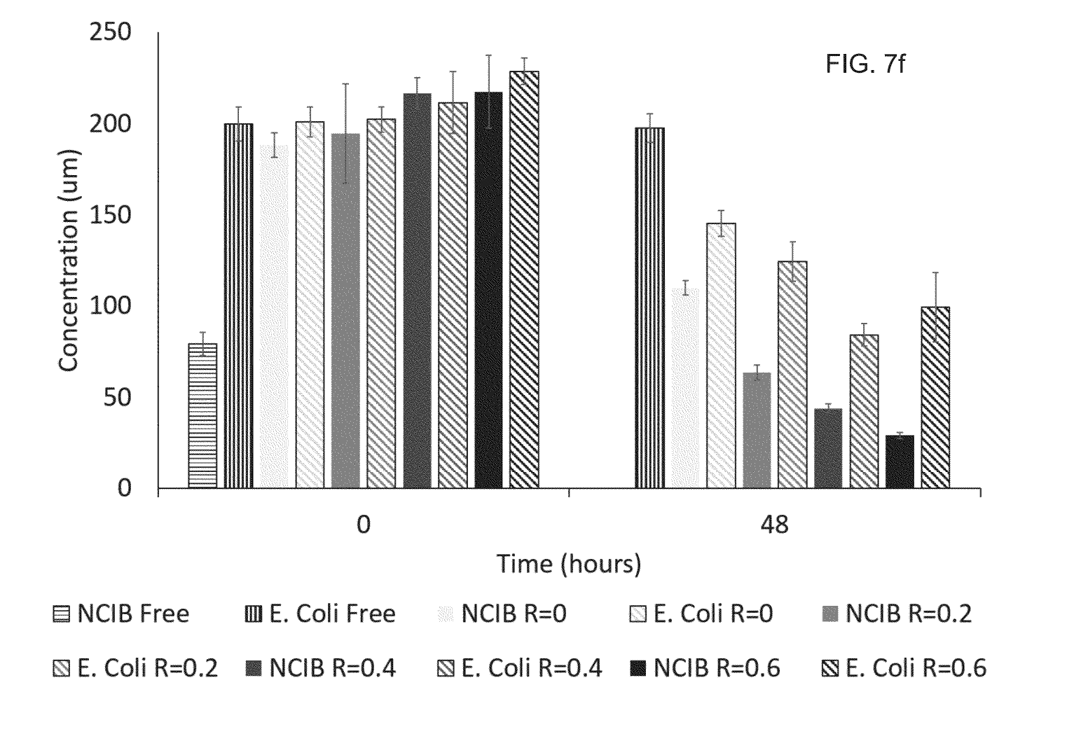

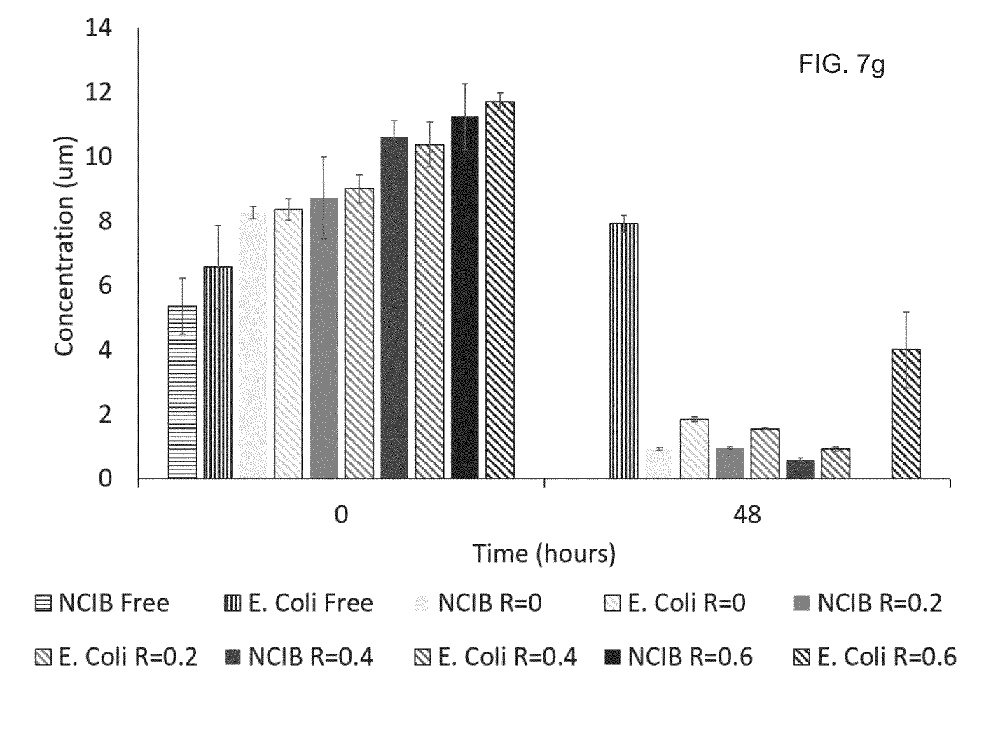

After incubation of the hydrocarbon solution for 48 hours, the concentration of naphthalene decreased significantly in all gel samples (FIG. 7). In the samples with encapsulated NCIB 9816, the removal of naphthalene increased with R, from a remaining concentration of 127.49±4.52 μM (R=0) to 36.10±2.16 μM (R=0.6). The samples with encapsulated E. coli DH5α followed a similar trend, with the concentration decreasing from 164.26±8.90 μM (R=0) to 107.67±19.69 μM (R=0.6), though there was no significant difference between R=0.4 and R=0.6. In the positive control with free NCIB 9816 cells, the concentration of naphthalene was below the detection limit of 1 nM, while the negative control (DH5α) showed no change in concentration over the time course, with a final concentration of 215.39±7.23 μM. Similar results were observed for), phenol (FIG. 7b), p-Cresol (FIG. 7c), indole (FIG. 7d), p-methoxyphenylmethyl sulfide (FIG. 7e), azulene (FIG. 7f), and phenathrene (FIG. 7g).

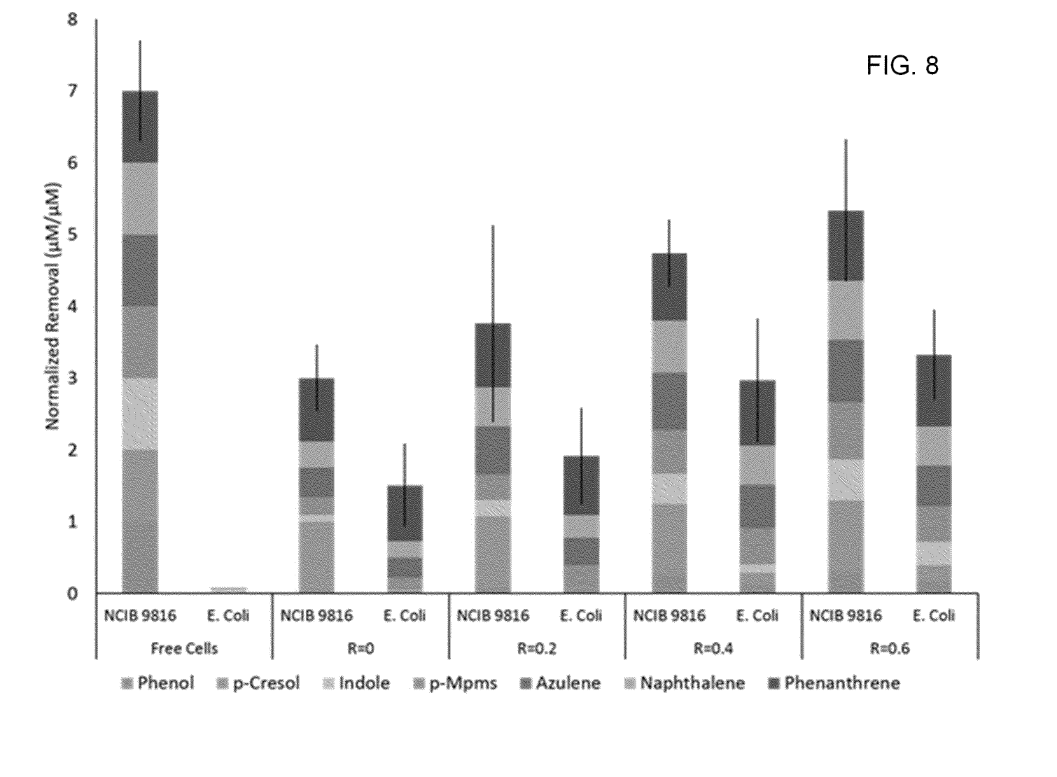

The combined removal of all hydrocarbon substrates from the solution showed increased removal for the more hydrophobic gels (FIG. 8). The results were normalized by the initial concentrations to account for any differences between the initial concentrations of the samples. The gels with biodegrading NCIB 9816 cells increased removal from 3.00±0.47 (R=0) to a maximum of 5.34±0.99 (R=0.6). The samples with non-degrading DH5α cells followed a similar trend, with the least removal of 1.51±0.58 (R=0) to a maximum of 3.33±0.63 (R=0.6). The positive control was 7.00±0.70 and the negative control was −0.37±0.42. The only substrate which was completely degraded was p-cresol and phenol had very low removal compared to the other substrates.

In this example, a series of hydrophobic silica gels containing encapsulated biodegrading bacteria were developed in order to facilitate removal by adsorption to the material and bioremediation by the encapsulated bacteria. The gel formulation used here was adapted from a previously developed method (Reátegui, E. et al. Silica gel-encapsulated AtzA biocatalyst for atrazine biodegradation. Appl. Microbiol. Biotechnol. 96, 231-40 (2012)). The silicon alkoxide precursors TMOS and MTMS were used in ratios (R) from 0 to 1, indicating the molar ratio of MTMS to total alkoxide. This example consisted of two parts: 1) Gel synthesis and characterization and 2) Application to a hydrocarbon mixture. The material characterization began by determining the gel hydrophobicity through water contact angle measurements and by using the fluorescent dye Nile Red, which is sensitive to hydrophobicity. The water contact angle measurements showed that the gel surface became more hydrophobic with increasing R, with a minimum of 7.3±1.0° (R=0) and a maximum of 98.0±1.5° (R=1).

Investigation of the gel microstructure revealed two distinct regimes: at R≦0.6, the microstructure was homogeneous, with pores ˜5 nm in size, whereas for R≧0.8, aggregates of particles ranging from 1-10 μm were observed (FIG. 4a). The formation and aggregation of large micro-particles (FIG. 4a, R=1) may be due to the reduced alkoxide functionality in MTMS and subsequently lower crosslinking density.

In this example, we observed maximum stress at fracture σ_{f}=1.1±0.1 MPa (R=0) for gels aged in PBS (FIG. 5a) and a minimum value of 16.43±3.45 kPa (R=1). Two regimes were also observed for the mechanical properties, with a ˜40% decrease in fracture stress from R=0 to R=0.6 to more than 90% decrease above R=0.8. The lack of homogeneous structure and inter-particle bonding (FIG. 4a, R=1) may correspond with the observed decrease in the mechanical properties in the gels with higher precursor ratio (R>0.6). The strength was lower for the gel with the highest hydrocarbon removal, 651.1±107.5 kPa (R=0.6), but this may be enhanced by drying in subsequent studies.

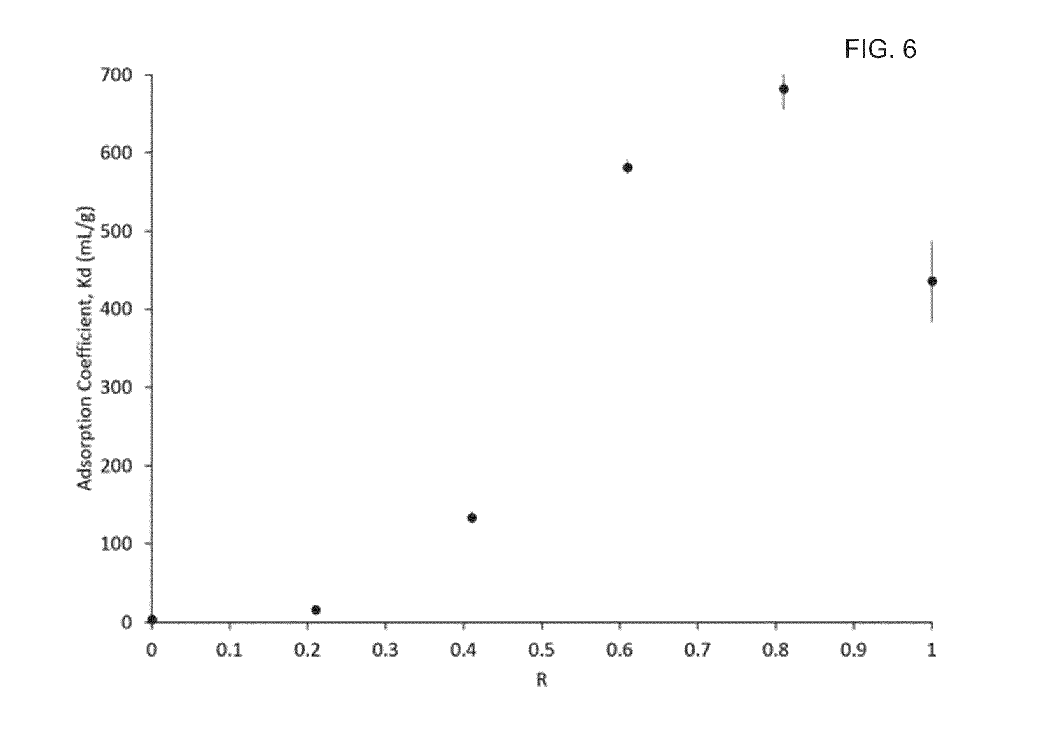

The equilibrium adsorption results (FIG. 6) indicated that the affinity for fluorene was increased exponentially from 4.44±2.62 mL/g (R=0) to a maximum of 681.88±26.21 mL/g (R=0.8). This increase in adsorption was expected, since a more hydrophobic surface (lower surface energy) should bind hydrophobic (low surface energy) chemicals. The reduced adsorption at R=1 of 435.85±52.25 mL/g may be due to reduced specific surface area as a result of the micro-particle formation.

The naphthalene results showed increased adsorption and total removal with increased gel hydrophobicity (FIG. 7a). The concentration of naphthalene left in the bulk solution (C/C₀) was ˜25%, indicating substantial but incomplete removal. A similar trend was observed in the combined removal results (FIG. 8). NCIB 9816 cells use a dioxygenase enzyme, naphthalene dioxygenase, for assimilation of the carbon from the substrates. Thus each step in the process requires one mole of molecular oxygen per mole of substrate. However, since the free NCIB 9816 cells were able to completely degrade all of the substrates, the oxygen levels in the vials should have been sufficient for degradation in the gels. Another explanation of the incomplete degradation might be that the cells were not viable/metabolically active long enough to remove the substrates completely. Finally, clogging of the pores with adsorbed substrates and/or products could be a third possible explanation for the observed results. Long-term degradation experiments may explain whether the cells were still active, while diffusion experiments could be used to explain any transport limitations that arise as a result of adsorption.

This example has shown that the removal of PAH by adsorption to the silica gel surface and bioremediation by encapsulated cells can be enhanced by increasing the hydrophobicity of the gel. For the gel with the best removal properties, the mechanical strength decreased about 30% from the maximum achieved with the hydrophilic gel (R=0), but was still mechanically stable. After R=0.6, the gels transitioned into agglomerates of micro-particles, which had very low mechanical strength. Further studies will be required to determine the longevity of the developed materials for use in flow-through systems.

### 2. Organic Modification of Silica Gels with Encapsulated Pseudomonas sp. NCIB 9816-4 for Enhanced Biodegradation of Aromatic Hydrocarbons

Silica gel matrices were made as discussed above in Example 1 (Remediation of PAH) in consideration of the following. Silicon alkoxides (Tetramethyl orthosilicate) or methyltrimethoxysilane) were added to 5 mM HCl, resulting in a final silicon alkoxide concentration of 3.4 M. The 0% gel was made with TMOS and the 50% gel used a 50% (mol/mol) TMOS/MTMS mixture prior to hydrolysis. The mixtures were stirred at room temperature for 2 hours to allow for hydrolysis. The hydrolyzed alkoxide solutions were mixed with colloidal silica nanoparticles (Ludox TM-40) and phosphate buffered saline (PBS) or cell suspension in a volumetric ratio of 2.5/2.5/1, respectively.

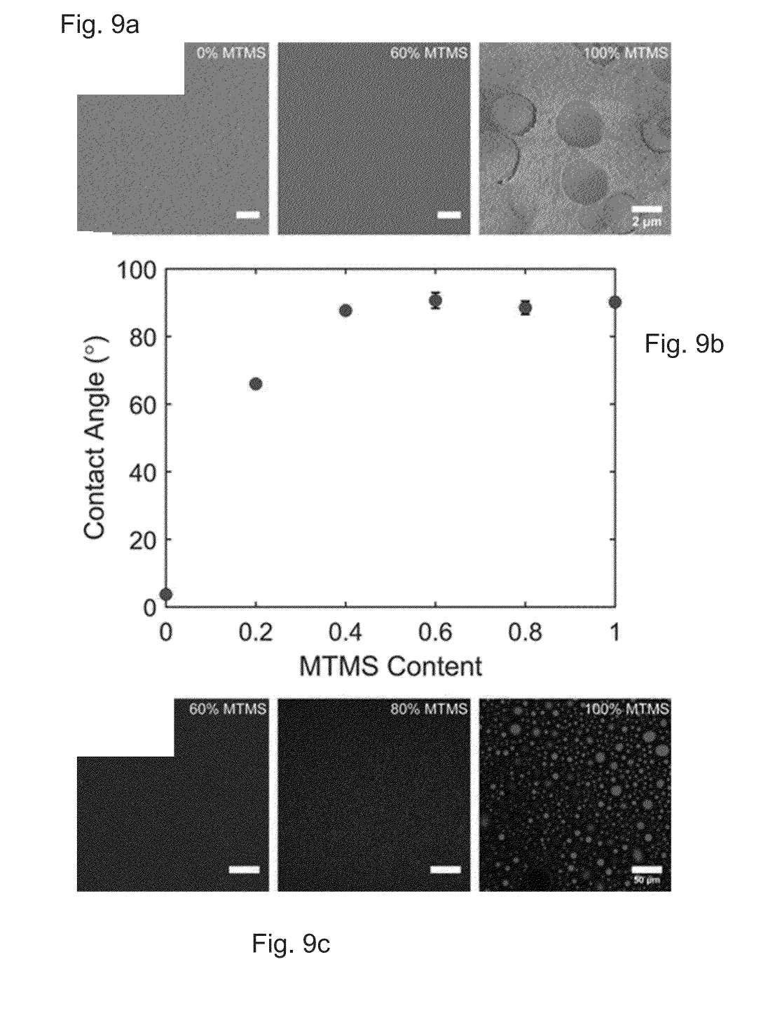

Surface and microstructural characterization was carried out using SEM, contact angle measurements and confocal microscopy. FIG. 9a shows the SEM images of the gel microstructure. The microstructure does not change much until very high concentrations of MTMS are utilized. At >80% MTMS, large spherical particles were observed forming in the gel structure, with diameters in the range of 1-10 μm. FIG. 9b shows the contact angle measurements. As seen there, the contact angle increases rapidly with the addition of MTMS to the gel composition and saturates above 40% MTMS, likely due to the high surface roughness of the gel, which includes 22 nm silica nanoparticles. FIG. 9c shows the confocal fluorescence images of the silica gels using the LUT to better illustrate the distribution of the hydrophobic methyl groups. As seen there, the distribution of the methyl groups is uniform at low concentrations of MTMS (≦60%), but forms aggregates/spherical particles at high MTMS concentrations (>60%).

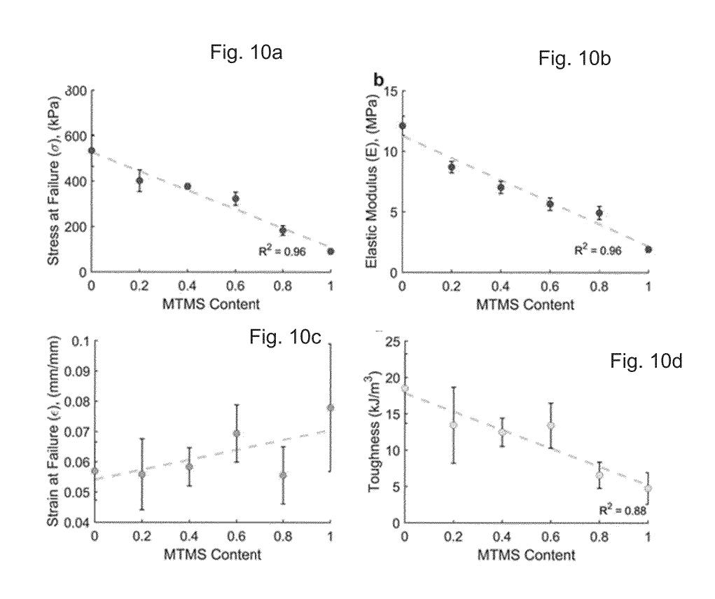

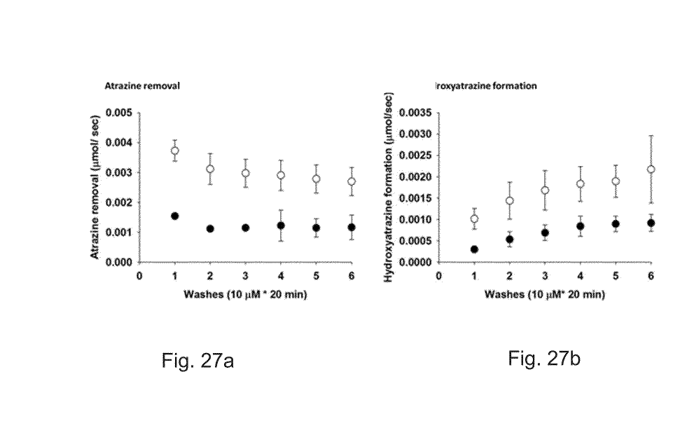

The mechanical properties of the gels were also determined by axial compression testing versus MTMS content. FIGS. 10a, 10b, 10c and 10d show the stress at failure versus MTMS content, the elastic modulus versus MTMS content, the strain at failure versus MTMS content and the toughness versus MTMS content respectively. As seen from a review of FIGS. 27a to 27b, the gel becomes a) weaker, b) more compliant, and d) less tough with increased MTMS. Based on the data presented in the FIGS. 9a to 9c and 10a to 10d, a gel with 50% MTMS was selected for further study in comparison to 0% MTMS as a hydrophilic gel.

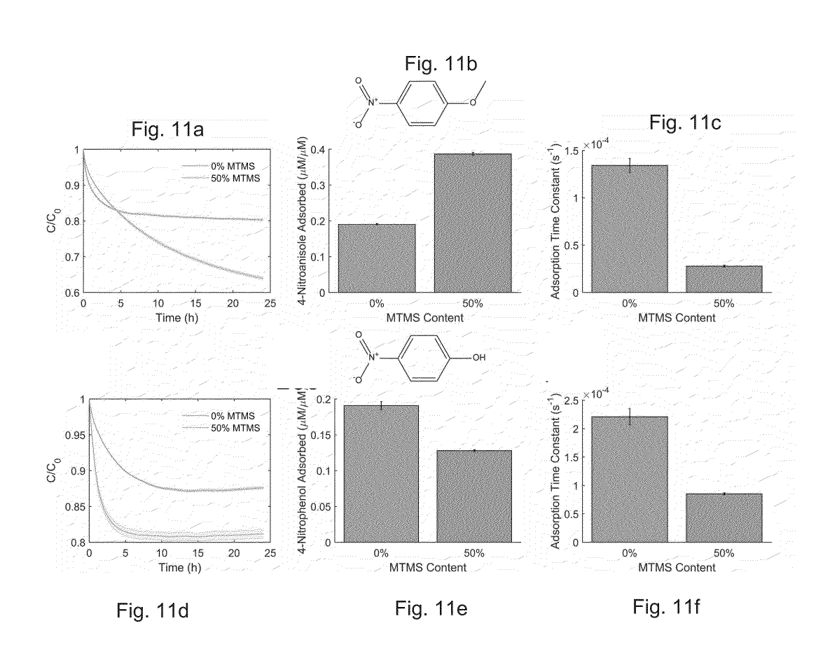

Adsorption characterization of 4-nitroanisole (FIGS. 11a-c) and 4-nitrophenol (FIGS. 11d-f) were evaluated with respect to the 50% MTMS gel and the 0% MTMS gel. Adsorption kinetics of each chemical was measured by UV-Vis over 24 hours (FIGS. 11a and 11d); relative amounts of each chemical adsorbed to the silica gel (FIGS. 11b and 11e); and time constant (c) of a model function fitted to the experimental data with the form y=a+be^{-ct} (FIGS. 11c and 11f) are reported herein.

As seen by comparing the figures, the hydrophilic gel (0% MTMS) adsorbed the 4-nitroanisole more quickly but to a lesser extent than the hydrophobic gel (50% MTMS). This may indicate a reduced diffusion coefficient in the hydrophobic gel when compared with the hydrophilic gel. In this case, the hydrophilic gel adsorbed more 4-nitroanisole than the hydrophobic gel and also had faster kinetics. If the results of both chemicals are compared, it becomes apparent that the hydrophilic gel adsorbs nearly the same about in both cases, with slightly faster kinetics for 4-nitrophenol. The hydrophobic gel, however, preferentially adsorbs 4-nitroanisole (more hydrophobic) while adsorbing less 4-nitrophenol (less hydrophobic). This may indicate that the hydrophobic gel allows selective partitioning of hydrophobic chemicals.

### 3. Remediation of Atrazine

In this example, a material, which has both a high adsorption capacity and enhanced biodegradation rates, was developed. A silica gel encapsulation method of Mutlu et. al (2013) (Mutlu, B. R.; Yeom, S.; Tong, H.-W.; Wackett, L. P.; Aksan, A., Silicon alkoxide cross-linked silica nanoparticle gels for encapsulation of bacterial biocatalysts. Journal of Materials Chemistry A 2013, 1, (36), 11051-11060) was utilized and incorporated hydrophobic functional groups in the gel to enhance hydrophobicity. The main hypothesis was that hydrophobic functional groups would enhance targeted adsorption of atrazine as well as facilitate its transport to the cell membrane, thus enhancing overall uptake and degradation. This is a promising new method for developing self-regenerating hybrid materials, which may have widespread application in water remediation technologies for a range of agro-chemicals, many of which are hydrophobic.

### Materials and Methods

Materials: The cross-linkers precursors used in the silica-gel preparation; Tetraethyl-orthosilicate (TeOs), triethoxy-methylsilane (TeMs), triethoxy-vinylsilane (TeVs) and triethoxy-phenylsilane (TePs) were purchased from Sigma-Aldrich (Sigma-Aldrich Corp. St. Louis, Mo., USA). The silica nanoparticles (Nex-sil 125-40, 80 nm diameter) were purchased from Nyacol (Nyacol Nano Technologies Inc., Ashland, Mass., USA). Technical grade atrazine and ametryn were provided by Syngenta (Syngenta Crop Protection, NC, USA). All other reagent used for buffers, HPLC solvents etc. were purchased from Sigma-Aldrich.

### Methods:

### Bacterial Growth Conditions:

The growth conditions of E. coli expressing AtzA enzyme have been described in detail previously (Mutlu, B. R.; Yeom, S.; Tong, H.-W.; Wackett, L. P.; Aksan, A., Silicon alkoxide cross-linked silica nanoparticle gels for encapsulation of bacterial biocatalysts. Journal of Materials Chemistry A 2013, 1, (36), 11051-11060) Briefly, E. coli DH5α (pMD4) were grown at 37° C. in superbroth medium (Mutlu, B. R.; Yeom, S.; Tong, H.-W.; Wackett, L. P.; Aksan, A., Silicon alkoxide cross-linked silica nanoparticle gels for encapsulation of bacterial biocatalysts. Journal of Materials Chemistry A 2013, 1, (36), 11051-11060) with vigorous aeration, supplemented with 50 μg mL⁻¹ chloramphenicol. Cells were harvested by centrifugation at 6000 rpm for 20 min and suspended at 1 g/mL in PBS.

### Silica Gel Preparation:

Hydrolysis and condensation reactions of silicon alkoxide (cross-linkers) were controlled by adjusting the water to alkoxide molar ratio and the solution pH as previously described by Mutlu et al. 2013 (Mutlu, B. R.; Yeom, S.; Tong, H.-W.; Wackett, L. P.; Aksan, A., Silicon alkoxide cross-linked silica nanoparticle gels for encapsulation of bacterial biocatalysts. Journal of Materials Chemistry A 2013, 1, (36), 11051-11060). The alkoxide to water molar ratio was set to 1:5.3:0.0013 (alkoxide:water:HCl), which according to previous literature results in a fully-hydrolyzed silicon alkoxide solution with a slow condensation rate (Mutlu, B. R.; Yeom, S.; Tong, H.-W.; Wackett, L. P.; Aksan, A., Silicon alkoxide cross-linked silica nanoparticle gels for encapsulation of bacterial biocatalysts. Journal of Materials Chemistry A 2013, 1, (36), 11051-11060). Seven different gels were prepared, one containing TeOs alkoxide only (hydrophilic gel) and the other gels with varying degrees of hydrophobicity. The three main gels were prepared by mixing the TeOs alkoxide with an alkoxide containing one functional hydrophobic group (either methyl, vinyl or phenyl) at a molar ratio of 1:1. Additionally, TePs gels with molar ratios of 1:3 (TeOs/TePs) and 100% TePs were also prepared. The hydrolysis procedure applied was the same for all solutions.

Next, the pH of the silica nanoparticles solution (concentration: 400 g/L) was adjusted to neutral pH by adding 1M hydrochloric acid. The silica gels were prepared by mixing 1.75 mL of the silica nanoparticles with 0.25 mL E. coli cells suspended in phosphate buffer saline (PBS) pH 7.4 (or just PBS when no cells were required) and 0.25 mL of the hydrolyzed cross-linker solution. The solutions were left to gel for 1.5 h. This resulted in silica gel plugs (volume: 2.25 mL with approximately 60% water content) formed at the bottom of 20 mL scintillation vials. All adsorption and activity experiments were carried out with these gel plugs.

### Contact Angle Measurements:

Samples were prepared by creating a 300 μL thin film of gel on the surface of a glass microscopy slide. Measurements were taken by a MCA-3 image analysis contact angle meter (Kyowa Science Interface Co., Japan). A 30 μm glass capillary tube was used to dispense distilled water and a static pressure of 15-30 kPa was applied for droplet generation. The reported contact angles were averaged from 10 droplet measurements performed in different locations on the sample.

### Scanning Electron Microscopy (SEM) Measurements:

Gel samples were prepared as described above and then gradually dehydrated in a series of ethanol washes (50, 70, 80, 95 and 100% EtOH). The ethanol was then evaporated off the samples overnight in the hood. Finally, the dried gel was put on a SEM carrier and sputter-coated with a thin layer of gold-palladium. SEM images were taken by a Hitachi S4700 machine. The high magnification image was achieved by using 3 kV with a distance of 3.3 mm between the beam and sample.

### Confocal Microscopy Measurements:

Samples were prepared on glass slides by depositing 300 μL of silica gel doped with 1 μg/mL Nile Red. Gels with cells were prepared by encapsulating E. coli expressing green fluorescent protein (GFP). Free cells with alkoxide samples were prepared similarly but without the addition of silica nanoparticles.

All measurements were carried out using a Nikon A1si confocal system equipped with a point-scan head, 5 standard PMT detectors and a 32-channel PMT spectral detector. The system is mounted on a Nikon Ti2000E inverted fluorescence microscope with DIC optics. Nile red was measured at an excitation wavelength of 561 nm and an emission range of 600-650 nm, GFP was measured at an excitation wavelength of 488 nm and an emission range of 500-550 nm. NIS Elements imaging software was used to control acquisition and analyze the images (including particle distribution calculations).

### Adsorption Isotherms:

Atrazine, ametryn and hydroxyatrazine (prepared by incubating AtzA with atrazine overnight) adsorption isotherms were carried out in 20 mL scintillation bottles. 3 mL of triazine solution (10-100 μM) were added to the different gels and left to agitate on a shaker overnight. The supernatant was then filtered through a 0.2 μm teflon filter and analyzed by HPLC.

The resulting plots were fitted to the Freundlich equation (Eq. 1), which relates the concentration of solute adsorbed on the surface (Y axis: C_{ads} (mmol/Kg)) to the concentration remaining in the solution (X axis: C_{eq} (mg/L)).

Freundlich equation: C_{ads}=k_{f}*C_{eq}ⁿ, k_{d}=k_{f}*C_{eq}⁽ⁿ⁻¹⁾ Eq. 1

Adsorption coefficients for the molecules (k_{d}) were then calculated at equal concentration for all compounds (10 μM).

### Kinetic Adsorption Measurements of Atrazine to the Gel and to Dry Powdered Gels:

Time dependent adsorption of atrazine to four gels (TeOs, 1:1 TePs/TeOs, 3:1 TePs/TeOs and TePs) was tested. Duplicate scintillation vials, with 10 mL atrazine solution (10 μM), were assigned for each time point, and the supernatant was extracted, filtered and analyzed by HPLC. To elucidate the role of the gel macro-structure, gels were also subjected to drying and crushing; this eliminates the diffusion through the gel macro-structure and exposes the specific functional groups. The gels, TeOs, 1:1 TePs/TeOs, 3:1 TePs/TeOs and TePs, were prepared as described and left to dry in the hood for three days. The dried gels were then thoroughly crushed with a pestle and mortar to a powder form. The powdered particles were then suspended in a 10 mL solution of atrazine (10 μM). The solutions were centrifuged (Eppendorf tubes, 14,000 RPM for 1 min) at different time points and analyzed by HPLC. It should be noted that all characterization techniques were done with cell-free gels.

### Activity Assays:

An atrazine solution (3 mL) was added to the selected gel plugs with active encapsulated bacteria for 20 min (Teos, TeOs/TeVs, TeOs/TePs (1:1, 1:2, 1:3) and TePs)). The solution was then separated from the gel, filtered and analyzed by HPLC. Following the solution separation a new solution containing fresh atrazine was immediately added to the gel for another 20 min. This procedure was repeated 8 times to reach pseudo steady state adsorption and degradation kinetics to best simulate a flow through reactor.

### Free Cell Activity Assays:

A solution of free cells was mixed with the cross-linker alone (without the silica nanoparticles) to evaluate the effect of the cross-linker on free cells (no gel formation). The ratios were the same as the gel activity ratios: 0.25 mL of E. Coli suspended cells and 0.25 mL of cross-linker solution were added to 1.75 mL solution of PBS. A 10 mL solution of atrazine was added in for 20 min and then the cells were separated by centrifuge in an Eppendorf tube (14,000 RPM for 1 min) and the supernatant was filtered through a 0.2 μm Teflon filter and analyzed by HPLC.

### HPLC Analysis:

The s-triazines were all analyzed using a Hewlett-Packard HP 1090 Liquid Chromatograph system equipped with a photodiode array detector. The detection method used an analytical C18 reverse-phase Agilent column at a wavelength of 220 nm, a H₂O/MeOH solvent ratio of 35%/65% and a flow rate of 1.0 mL/min.

### Results

### Characterization of Silica Gels:

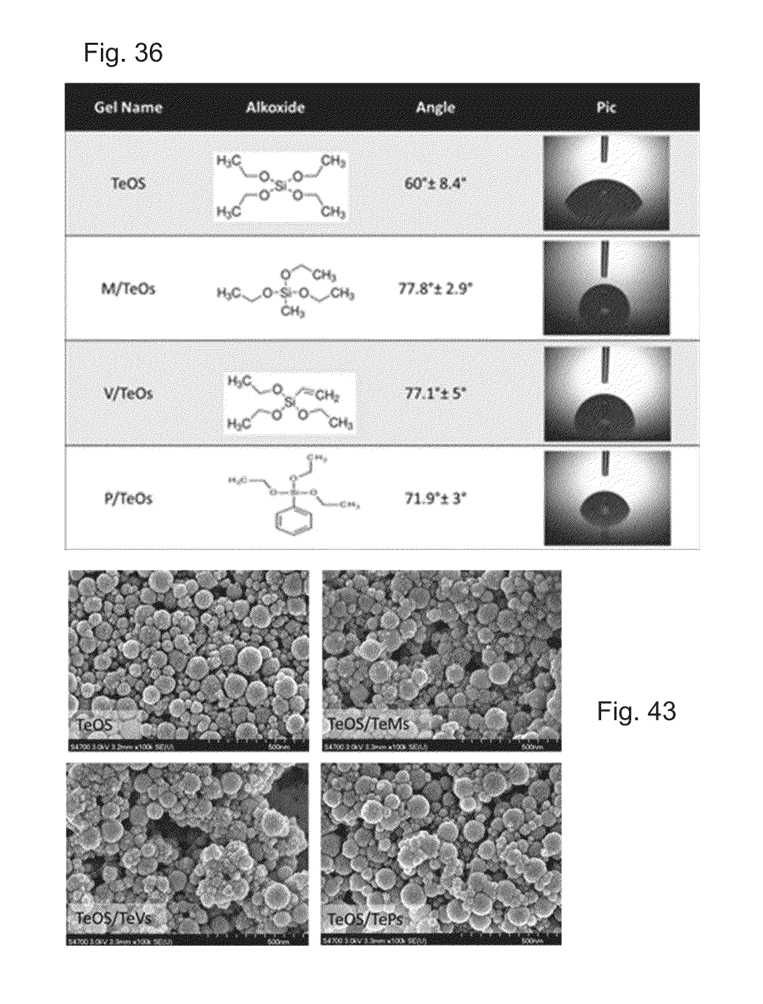

The current study focused on increasing hydrophobicity of the silica encapsulation matrix in order to enhance both adsorption and degradation kinetics. A hydrophilic, non-adsorbent gel was chosen as a baseline for comparison and was prepared as previously described by Mutlu et. al 2013. This gel was composed of silica nano-particles cross linked by TeOs. As stated in previous studies, the incorporation of larger silica nano-particles increases pore size and diffusional properties of the gel matrix (Mutlu, B. R.; Yeom, S.; Tong, H.-W.; Wackett, L. P.; Aksan, A., Silicon alkoxide cross-linked silica nanoparticle gels for encapsulation of bacterial biocatalysts. Journal of Materials Chemistry A 2013, 1, (36), 11051-11060). Three different hydrophobic functional groups were incorporated in the baseline silica-gel matrix and were compared in respect to adsorption and degradation. These modified gels were prepared by using a mixture of the TeOs cross-linker with cross-linkers containing one functional hydrophobic group (methyl (TeMs), vinyl (TeVs) or phenyl (TePs)) at a molar ratio of 1:1 (FIG. 36)

The four gels were then characterized in terms of hydrophobicity by contact angle measurements, SEM and confocal fluorescence microscopy (using Nile red dye) and in terms of adsorption capability by adsorption/desorption isotherms.

### Contact Angle Measurements.

Contact angle measurements give an indication of the level of hydrophobicity and surface roughness of the silica gels (Hegde, N. D.; Venkateswara Rao, A., Organic modification of TEOS based silica aerogels using hexadecyltrimethoxysilane as a hydrophobic reagent. Applied Surface Science 2006, 253, (3), 1566-1572; Venkateswara Rao, A.; Kalesh, R. R., Comparative studies of the physical and hydrophobic properties of TEOS based silica aerogels using different co-precursors. Science and Technology of Advanced Materials 2003, 4, (6), 509-515; Kros, A.; Gerritsen, M.; Sprakel, V. S. I.; Sommerdijk, N. A. J. M.; Jansen, J. A.; Nolte, R. J. M., Silica-based hybrid materials as biocompatible coatings for glucose sensors. Sensors and Actuators B: Chemical 2001, 81, (1), 68-75; Vigil, G.; Xu, Z.; Steinberg, S.; Israelachvili, J., Interactions of silica surfaces. Journal of Colloid and interface science 1994, 165, (2), 367-385; and Wang, M.; Chen, C.; Ma, J.; Xu, J., Preparation of superhydrophobic cauliflower-like silica nanospheres with tunable water adhesion. Journal of Materials Chemistry 2011, 21, (19), 6962-6967). The contact angle of TeOs based gels reported in literature varies significantly (from 38° (Kros, A.; Gerritsen, M.; Sprakel, V. S. I.; Sommerdijk, N. A. J. M.; Jansen, J. A.; Nolte, R. J. M., Silica-based hybrid materials as biocompatible coatings for glucose sensors. Sensors and Actuators B: Chemical 2001, 81, (1), 68-75) to 980 (Hegde, N. D.; Venkateswara Rao, A., Organic modification of TEOS based silica aerogels using hexadecyltrimethoxysilane as a hydrophobic reagent. Applied Surface Science 2006, 253, (3), 1566-1572)), our measurements showed a contact angle of 60°. The somewhat high angle (compared to the literature) could be due to high siloxane (Si—O—Si) areas without silanol groups (O—Si—OH), or partial hydrolysis of the cross-linker leaving behind some of the initial orthosilicate groups (Si—O—CH₂—CH₃) trapped within the gel (Hegde, N. D.; Venkateswara Rao, A., Organic modification of TEOS based silica aerogels using hexadecyltrimethoxysilane as a hydrophobic reagent. Applied Surface Science 2006, 253, (3), 1566-1572; and Vigil, G.; Xu, Z.; Steinberg, S.; Israelachvili, J., Interactions of silica surfaces. Journal of Colloid and interface science 1994, 165, (2), 367-385). A significant change in the contact angle was observed between the hydrophilic, TeOs based, gel and the other three modified gels (FIG. 36). The highest contact angle was observed for the methyl functionalized gel, however, results are comparable within error for all hydrophobic gels. Hedge and Rao (2006) observed similar results for aerogels based on TeOs mixed with a variety of functionalized cross-linkers (60% molar ratio). The contact angle was found to be the highest for the methyl cross-linker (136°), while for the other cross-linkers (i.e. ethyl and phenyl) the angle ranged between 120° and 130° (Hegde, N. D.; Venkateswara Rao, A., Organic modification of TEOS based silica aerogels using hexadecyltrimethoxysilane as a hydrophobic reagent. Applied Surface Science 2006, 253, (3), 1566-1572). The lower contact angle observed in this study, compared to Hedge and Rao, is due to the gel matrix being composed mainly of hydrophilic silica nano-particles as well as the cross-linker (7:1 w/w respectively).

Contact angle measurements showed the change in overall hydrophobicity of the gel but were not able differentiate between the functional groups (the only additional difference observed was that the water contact angle for 1:1 TeOs/TePs gel (phenyl functionalized gel) was difficult to measure due to the water droplet tendency to slide off the silica gel surface. The behavior of the water droplet could indicate phase separation; resulting in hydrophobic and hydrophilic patches within the gel.

In order to further investigate the properties of the functionalized gels, two different microscopic techniques were utilized: SEM to explore the structural differences among the gels (see FIG. 43), and confocal fluorescence microscopy to measure the size and the degree of homogeneity of the hydrophobic areas generated within each gel. The bigger particles in the images are most likely the silica 80 nm particles and the smaller particles are the cross-linker (alkoxide) particle aggregates. From the images it appears that the mixing of two types of alkoxides (regardless of the functional group) creates a rougher and more porous macrostructure, which is in agreement with the contact angle measurements (rougher surface area, higher contact angle). However, these are only representative images and cannot be used to derive statistically significant conclusions about surface roughness and apparent porosity.

### Confocal Fluorescence Microscopy:

Nile Red dye was used as a hydrophobicity probe based on the well-known property to fluoresce in non-polar environments. In agreement with previous studies (Khamova, T. V.; Shilova, O. A.; Movchan, T. G.; Sazhnikov, V. A.; Rusanov, A. I., Sol-gel synthesis and fluorescence properties of hybrid nanocomposite materials doped with the Nile Red dye. Glass physics and chemistry 2008, 34, (1), 63-67; Lobnik, A.; Wolfbeis, O. S., Probing the polarity of sol-gels and ormosils via the absorption of Nile Red. Journal of sol-gel science and technology 2001, 20, (3), 303-311; and Fu, Y.; Ye, F.; Sanders, W. G.; Collinson, M. M.; Higgins, D. A., Single molecule spectroscopy studies of diffusion in mesoporous silica thin films. The Journal of Physical Chemistry B 2006, 110, (18), 9164-9170) the fluorescence spectroscopy and confocal fluorescence measurements revealed significant changes in the overall emission intensities and structural characteristics of the gel as a function of the alkoxide used, thus enabling differentiation between the different functional groups (FIG. 11A). As hypothesized, fluorescence increased as a function of hydrophobicity and alkoxide functional group size, in the following order: non-functionalized<methyl<vinyl<phenyl. This is due to the stronger fluorescence of the Nile red dye with the non-polar functional groups (Khamova, T. V.; Shilova, O. A.; Movchan, T. G.; Sazhnikov, V. A.; Rusanov, A. I., Sol-gel synthesis and fluorescence properties of hybrid nanocomposite materials doped with the Nile Red dye. Glass physics and chemistry 2008, 34, (1), 63-67; and Lobnik, A.; Wolfbeis, O. S., Probing the polarity of sol-gels and ormosils via the absorption of Nile Red. Journal of sol-gel science and technology 2001, 20, (3), 303-311).

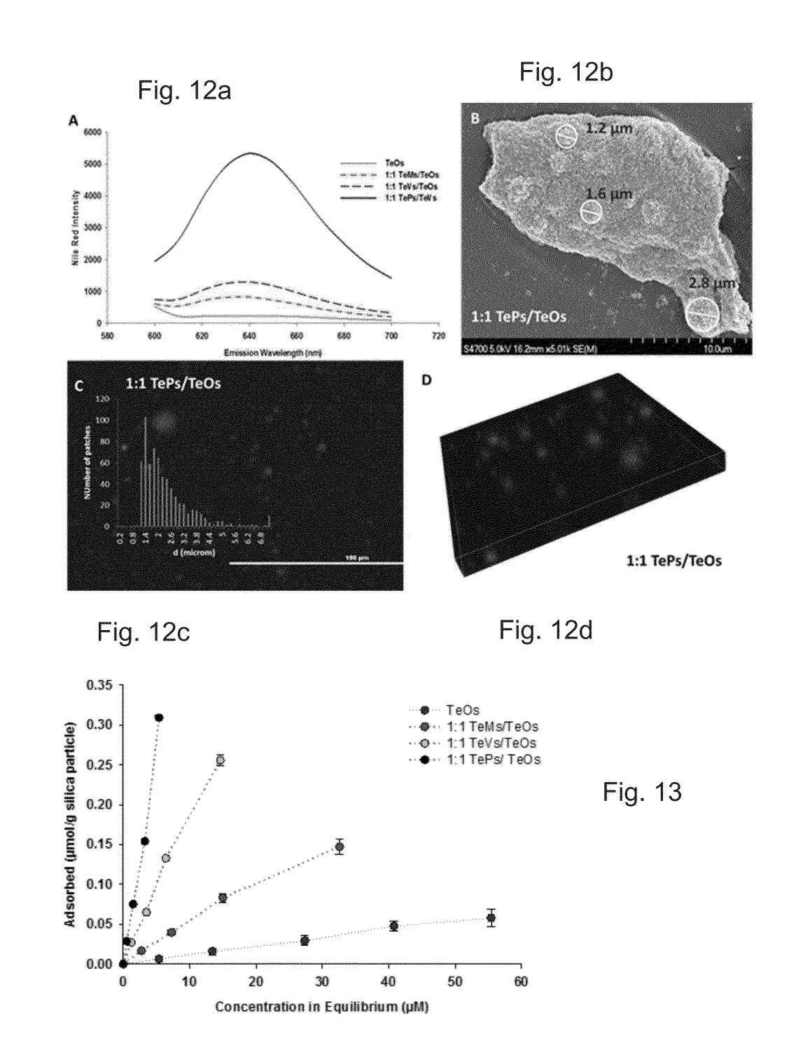

The confocal image of the 1:1 TePs/TeOS gel (FIGS. 12c/12d) displays the formation of hydrophobic areas within the gel as indicated from the water droplet behavior in the contact angle measurements. The red fluorescent patches (hydrophobic patches) vary in size, ranging in diameter from 0.8-3 μm (FIG. 12c), and occupy roughly 7% of the total gel observed area in the image. Similar sized patches averaging 1.6 μm, were also identified in SEM images; an estimate of the occupied area gives comparable 5-6% coverage (FIG. 12b). It is thought, but not relied upon that these hydrophobic aggregates may create favorable binding sites for the atrazine molecules, in turn enhancing the adsorption capacity of the gel.

Adsorption of Atrazine to the Silica Gels with the Four Selected Cross-Linkers:

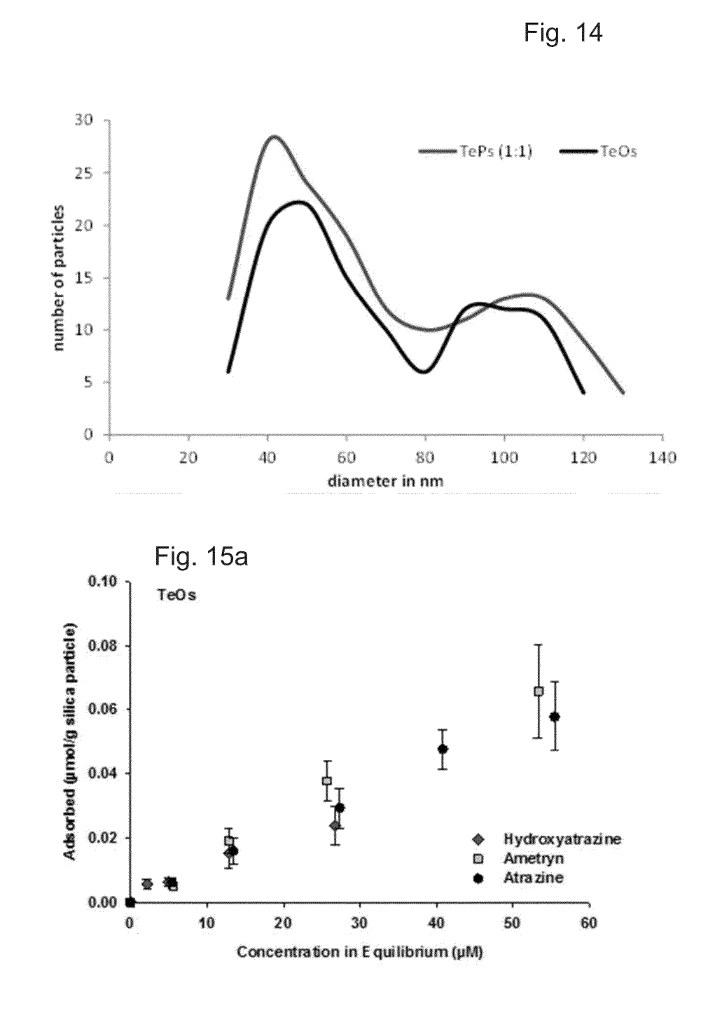

Following the initial characterization of the gels, adsorption isotherms of atrazine to the different gels were constructed (FIG. 13). In agreement with the Nile red fluorescent experiments, the affinity of atrazine to the gels increased as a function of functional group size and hydrophobicity in the following order: non-functionalized<methyl<vinyl<phenyl. More than 90% removal of atrazine from the solution over 24 h was observed in the 1:1 TeOs/TePs gel and in contrast, the TeOs gel reached less than 15% removal. Furthermore, desorption experiments conducted on all four gels (FIG. 14) showed hysteresis in the same order, which means the release of atrazine from the gel decreases as a function of gel hydrophobicity. This further suggests a more specific adsorption mechanism.

### Preferential Adsorption of Hydrophobic Compounds to the Functionalized Gels:

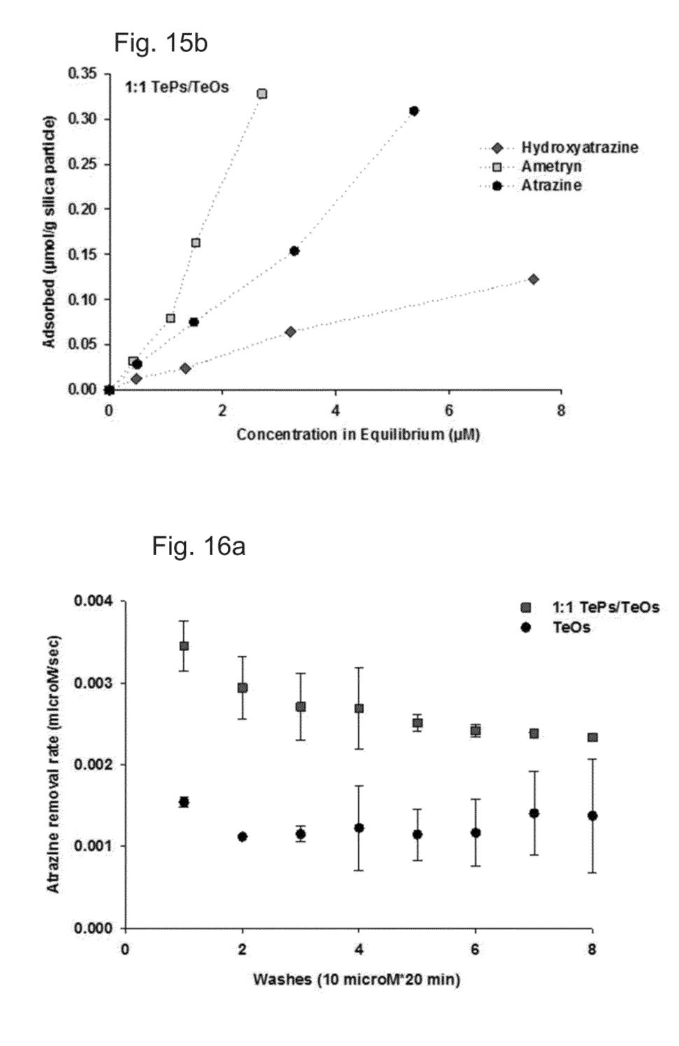

In order to establish the effect of the compound hydrophobicity on adsorption behavior, the adsorption of three s-triazine compounds (hydroxyatrazine, atrazine, and ametryn), with a range of log k_{ow} values (and hence hydrophobicities) were tested with the TeOs, 1:1 TeVs/TeOs and 1:1 TePs/TeOS gels. Adsorption isotherms were constructed and fitted to the Freundlich equation in order to extract comparable binding coefficients. The results from the TeOs and 1:1 TePs/TeOs silica-gels are plotted in FIGS. 15a and 15b and the fitted coefficients for all three gels are summarized in Table 2 below.

The adsorption behavior of all three compounds to the TeOs gel was low (˜15%) and similar (within error). This implies that their adsorption mechanism is not affected by their hydrophobic properties. Since the silica gel matrix should be chemically inert, we suggest the mechanism to be simple physical trapping within small sized pores in the gel. In contrast, the adsorption of the compounds to 1:1 TeVs/TeOs and 1:1 TePs/TeOs correlated to the compounds' log k_{ow} with a more pronounced difference in adsorption behavior as a function of hydrophobicity in the 1:1 TePs/TeOs gel. The adsorption was in the order of hydroxyatrazine (k_{d}=0.016)<atrazine (k_{d}=0.063)<ametryn (k_{d}=0.2). This is a desirable property for systems that integrate adsorption and degradation, because the affinity of the substrate (atrazine) to the matrix is four times higher than that of the product (hydroxyatrazine). Consequently, we would expect new atrazine molecules diffusing into the gel to out-compete the product; this will improve adsorption capacity as well as degradation efficiency. Since pesticide degradation products are generally more polar than the pesticides themselves, this is a desirable property for many biodegradation applications.

TABLE 2
s-Triazine adsorption isotherm Freundlich binding coefficients

| Gel type | s-Triazine | k_{f} | n | k_{d} (10 μM) |
|---|---|---|---|---|
| TeOs | Hydroxyatrazine | 0.0027 | 0.67 | 0.0013 |
| | Atrazine | 0.0014 | 0.93 | 0.0012 |
| | Ametryn | 0.0019 | 0.89 | 0.0015 |
| 1:1 TeVs/TeOs | Hydroxyatrazine | 0.0053 | 0.97 | 0.005 |
| | Atrazine | 0.024 | 0.88 | 0.018 |
| | Ametryn | 0.024 | 1.05 | 0.026 |
| 1:1 TePs/TeOs | Hydroxyatrazine | 0.023 | 0.85 | 0.016 |
| | Atrazine | 0.041 | 1.18 | 0.063 |
| | Ametryn | 0.085 | 1.36 | 0.2 |

### Adsorption and Degradation of Atrazine by the Functionalized Gels:

The degradation and adsorption of atrazine were measured by following the removal of atrazine from the solution (by adsorption and degradation) along with the formation of hydroxyatrazine (the product of degradation). The bio-reactive gels were incubated for 20 min with atrazine solution; the solution was then removed for analysis and the gels were immediately reintroduced to a fresh atrazine solution for another 20 min. This procedure was repeated until a pseudo steady-state (no significant change in rate was observed over three washes) for adsorption and degradation of atrazine was reached.

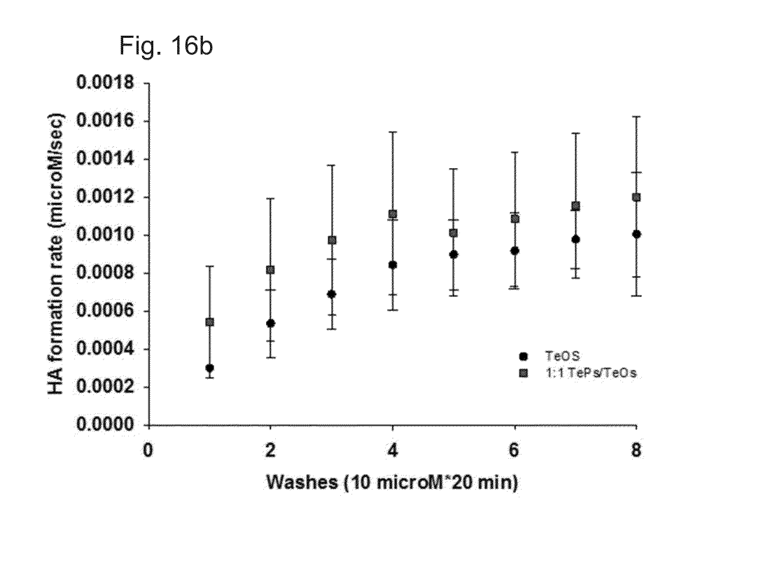

Two of the gels were tested: the non-adsorbent TeOs gel and the most hydrophobic 1:1 TePs/TeOs gel. The results were consistent with the previous adsorption experiments, showing significantly higher atrazine removal rates for the functionalized gel compared to the TeOs gel (FIG. 16a). The removal of atrazine by the TeOs gel reached a pseudo steady-state after 2 washes and the amount of hydroxyatrazine formed was equal to the amount of atrazine removed (showing no adsorption—FIG. 16b). On the other hand, the 1:1 TePs/TeOs gel did not reach adsorption saturation and even after 8 washes the amount of hydroxyatrazine formed was still lower than the amount of atrazine removed.

These results suggest that the adsorption and degradation processes, although simultaneous, do not affect each other. One possible explanation is that the hydrophobic patches are relatively sparse within the gel matrix and these behave as atrazine binding sites where bacteria feed, yet they do not change the rate of diffusion into the cell membrane. Hence, the removal rate is high, but the concentration of diffused molecules in the hydrophilic bulk of the gel is comparable to that of the TeOs gel. It should be noted that in both gels, solutions left for 24 h showed complete atrazine removal, freeing up new adsorption and degradation sites.

Enhanced Activity of Encapsulated E. coli:

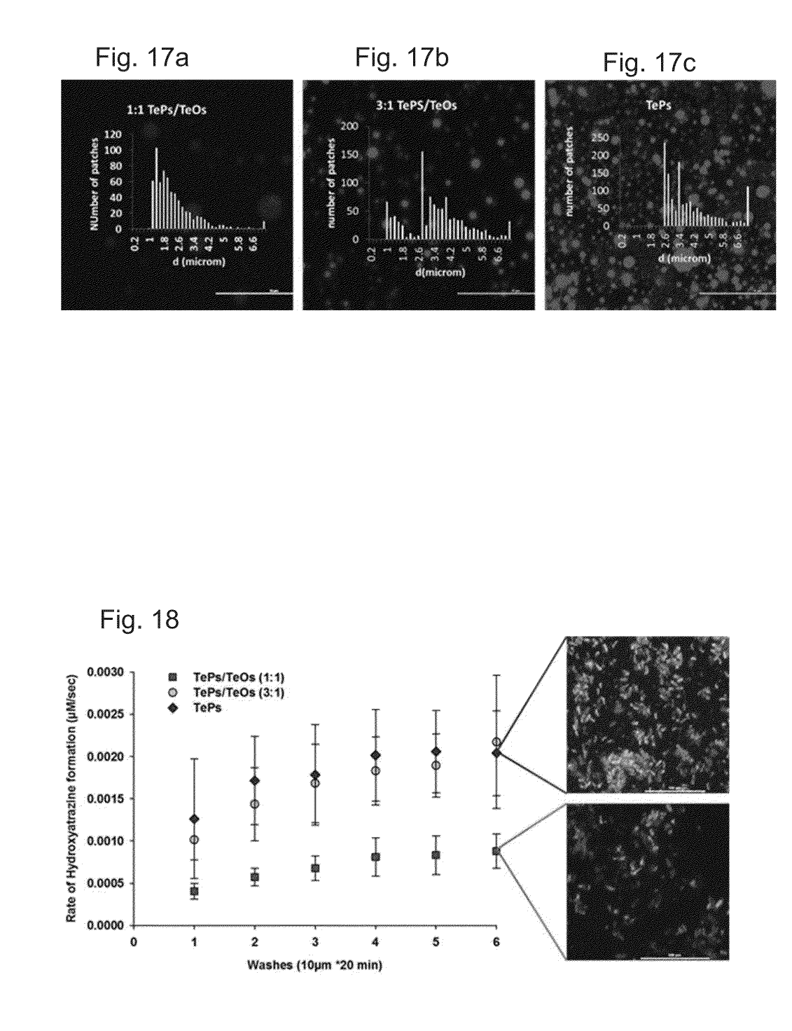

Two more gels with increased TePs to TeOs ratio were made: TePs/TeOs (3:1 molar ratio) and TePs (100%). The gels were examined with confocal microscopy (with Nile red) to see if there was any change in the fluorescence pattern; the size and density of the patches were assessed. The increase in TePs/TeOs ratio resulted in a higher density of hydrophobic areas within the gels (FIGS. 17a, 17b, and 17c). Confocal measurements of the gels with encapsulated GFP also showed differences in terms of gel fluorescence and cell arrangement (FIG. 18). In all phenyl functionalized gels the cells seem to aggregate around the hydrophobic areas, yet in the higher TePs/TeOs ratio gels this is more noticeable due to the higher density of hydrophobic patches.

The change in the TePs/TeOs ratio led to a significant increase in the degradation rates of atrazine by the encapsulated E. coli (FIG. 6). The degradation increased in a stepwise manner; above a certain threshold of hydrophobicity, the degradation rate increases three-folds. However, no significant change in atrazine removal rates was observed between these gels (results not shown). These results suggest that an increase in phenyl cross-linker changes the gel structure in a way that facilitates atrazine transport into the cell. In these gels adsorption and degradation rates are nearly the same, meaning the atrazine is not sequestered but is available for uptake by the bacteria (unlike the 1:1 TePs/TeOs).

Two hypotheses were examined to elucidate the enhanced degradation phenomenon; 1) the highly hydrophobic cross-linker increases the hydrophobic patch density thus facilitating atrazine diffusion into/throughout the gel and to the cells, 2) TePs at high concentrations affects either the cell permeability or the arrangement of bacteria in the gel which results in increased degradation rates.

Diffusion/Adsorption of Atrazine to Gels with Different TePs/TeOs Ratios:

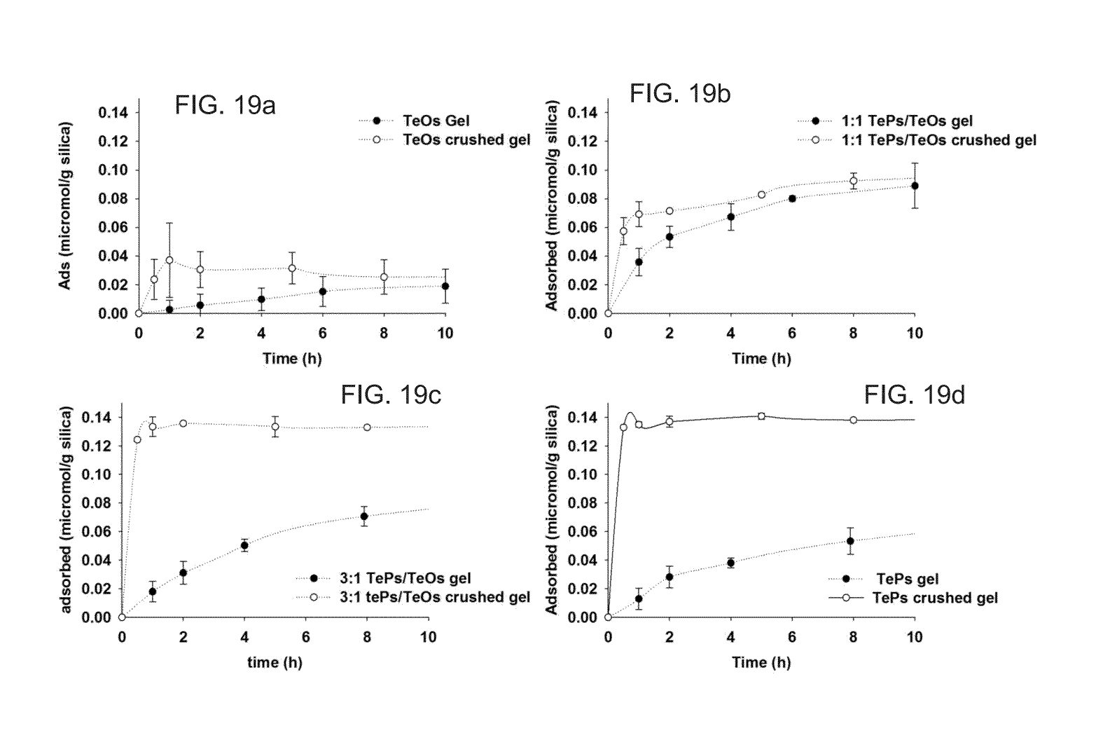

The first step in elucidating the mechanism was to evaluate whether the cross-linker ratio affected the diffusivity of atrazine into and throughout the gel. To answer this question, the kinetics of atrazine adsorption to the wet gel (plug form) in comparison to the same gel dried, crushed to a fine powder and suspended, were measured. The gel was dried and crushed to eliminate the macrostructure, leaving only diffusion and adsorption to the nano/microparticles of the gel. The results (FIGS. 19a to 19d) suggest that a change in the gel nanostructure does occur in the range between 50% (1:1)-75% (3:1) ratio of TePs (showing step-function behavior). A significantly higher difference in adsorption is noticeable with the high TePs content gels (3:1 TePs/TeOs and 100% TePs). This suggests that the adsorption to, and diffusion through the nanoparticles increases, yet the diffusivity into the macrostructure of the wet gel decreases in the high TePs ratio gels (or is a kinetic bottleneck), otherwise the removal rates in the gel would be higher and not comparable to the 1:1 TePs/TeOs gel.

### Effect of Cross-Linker on Free Cell Solution:

The hydrolyzed cross-linker solutions (250 μL of TeOs, 1:1 TePs/TeOs, 3:1 TePs/TeOs and TePs) were added to a solution containing atrazine and E. coli cells expressing AtzA. In these suspensions, the free cells were not encapsulated but were suspended alongside the cross-linker aggregates; this eliminates the diffusion of atrazine through the gel matrix. Upon addition of the cross-linker, the solutions became cloudy and aggregation of particles was noticeable. Results of atrazine removal and hydroxyatrazine formation are displayed in FIG. 8. The trends in atrazine adsorption and degradation for free cell with cross-linker solutions were the same as for the gel plugs: atrazine removal increases significantly between the TeOs and 50% (1:1) TePs/TeOS and then remains constant, and degradation increased significantly at 75% (3:1) TePs/TeOS and remained the same for the 100% TePs cross-linked gel. It should be noted that the non-hydrolyzed cross-linker did not affect adsorption or degradation and atrazine removal rates were comparable to those in a free cell solution with no additives. This suggests that the cells are not affected by the phenyl groups and it is the cross-linker nanoparticle formation (both in solution and in the gel) that governs the adsorption and degradation rates.

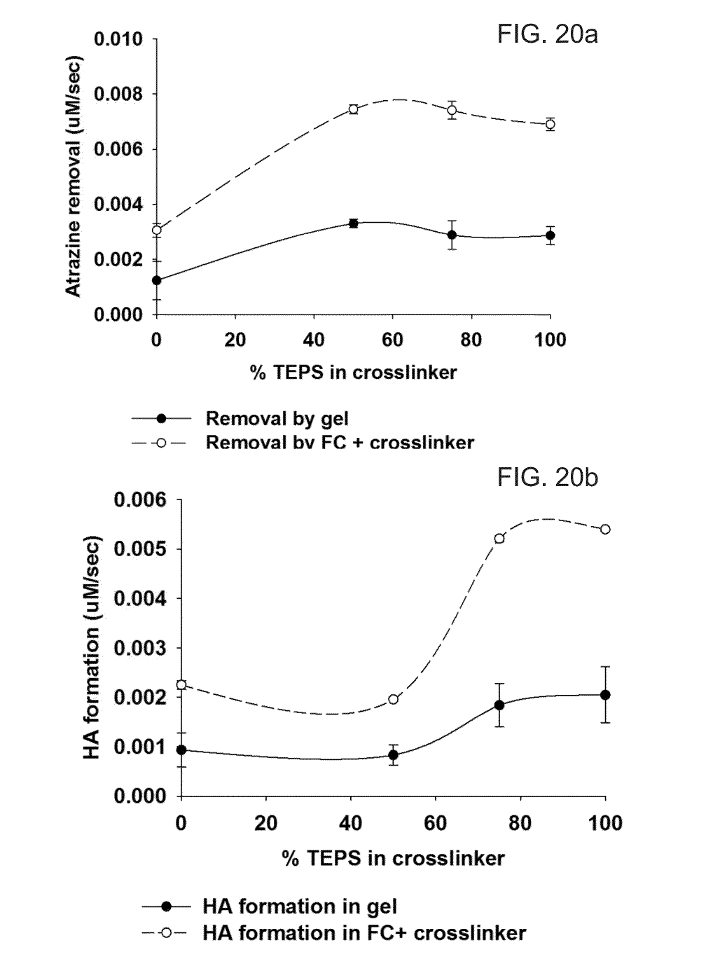

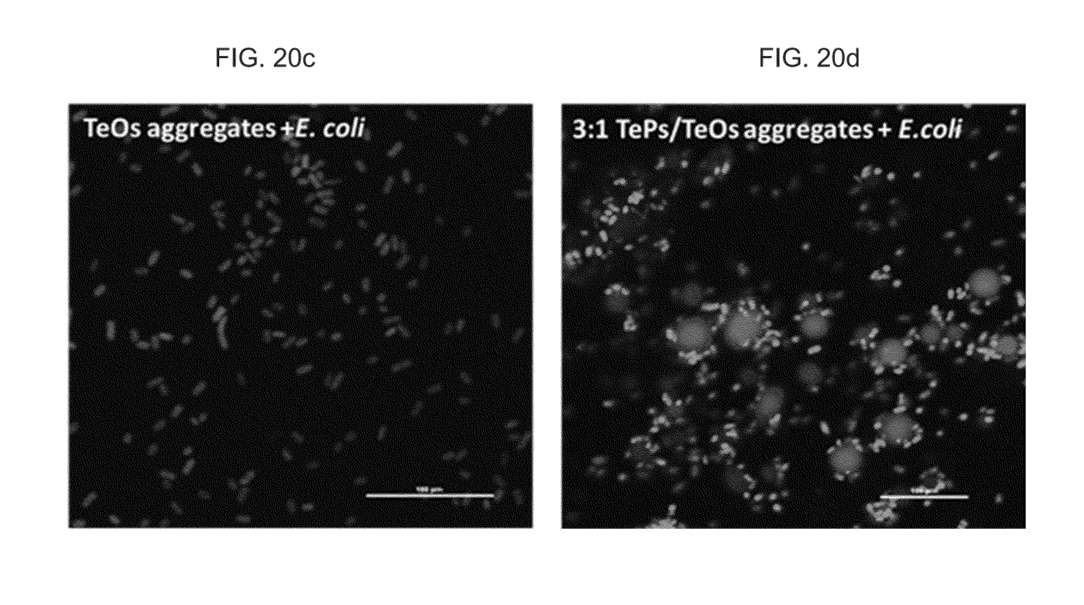

To complement these observations, confocal images of free cells (E. coli expressing GFP) and cross-linker solutions were obtained (FIG. 20B). The results show that the bacteria adhere to the phenyl functionalized particles (both 1:1 TePs/TeOs and 3:1 TePs/TeOs) whereas the cells mixed with the TeOs aggregates are somewhat dispersed. See FIGS. 20a to 20d generally. This presents a unique configuration where the substrate and the bacteria feeding on it, adhere to the same area, allowing rapid uptake of the substrate.

Bacterial adhesion to hydrophobic surfaces has been reported in the past (Arai, T.; Norde, W., The behavior of some model proteins at solid-liquid interfaces; Adsorption from single protein solutions. Colloids and Surfaces 1990, 51, 1-15; and Norde, W.; Lyklema, J., Protein adsorption and bacterial adhesion to solid surfaces. Colloids and Surfaces 1989, 38, 1-13) however, this phenomenon, where the 3-dimensional encapsulation structure controls both substrate removal and bacterial activity has not, to the best of our knowledge, been observed. The results conveyed in FIGS. 19a to 19d and 20a to 20d do not fully explain the adsorption and degradation step-function behavior, suggesting that the mechanism is more complex. The gel inner-structure and the encapsulated cell arrangement should therefore be the subject of further inquiry.

In summary, hydrophobic-biodegrading gels with an ability to efficiently remove and degrade atrazine were developed and tested. Highly hydrophobic gels exhibited not only high adsorption capabilities but also preferential affinity for atrazine (substrate) over hydroxyatrazine (product). At a molar ratio of 1:1 TePs/TeOs cross-linker ratio, activity assays resulted in enhanced atrazine adsorption and comparable degradation rates. A further increase in the TePs/TeOs ratio significantly enhanced the degradation rate as well. We suggest that this results from a change in the gel inner-structure that affects atrazine transport to the cells. Deciphering the nanostructure of the cross-linker aggregates and its effect on bio-reactivity can be extremely beneficial not only for water remediation but for any biocatalysis system. This allows fine-tuning of the gel to enable differentiation between substrates or to aid in substrate/product purification—thus substantially improving turnover efficiency. Therefore, our future studies will focus on the effects functional organic groups have on the silica-gel internal structure and how these structures change the bacterial microenvironment and arrangement.

### 4. Enhanced Degradation of Atrazine with Less Binding of Hydroxyatrazine by Phenyl-Silica Matrix

### Materials and Methods

Chemicals: The two cross-linkers precursors that were used in the silica-gel preparation, tetraethoxysilane (Teos), triethoxy-methylsilane (Mtos), triethoxy-vinylsilane (Vtos) and triethoxy-phenylsilane (Ptos) were purchased from Sigma-Aldrich (Sigma-Aldrich Corp. St. Louis, Mo., USA). phenytriethoxylsilane (Ptos), were purchased from Sigma-Aldrich (Sigma-Aldrich Corp. St. Louis, Mo., USA). The silica nanoparticles (Nex-sil 125-40, 80 nm diameter) were purchased from Nyacol (Nyacol Nano Technologies Inc., Ashland, Mass., USA). Technical grade atrazine and ametryn were provided by Syngenta (Syngenta Crop Protection, NC, USA). All other reagent used for buffers, HPLC solvents etc. were purchased from Sigma-Aldrich.

Bacterial growth conditions: The growth conditions of E. coli expressing AtzA enzyme have been described previously. Briefly, E. coli DH5α (pMD4) were grown at 37° C. in superbroth medium with vigorous aeration, supplemented with 30 μg mL⁻¹ chloramphenicol. E. coli DH5α expressing green fluorescent bacteria (GFP), transformed as previously described, were grown at 37° C. in LB medium with vigorous aeration, supplemented with 50 μg mL⁻¹ kanamycin. The cells were harvested by centrifugation at 6000 rpm for 20 min and suspended at 1 g/mL in phosphate saline buffer (PBS).

Silica gel preparation: Silica gels were prepared by mixing hydrolyzed alkoxides as cross linkers with a solution of 80 nm silica nanoparticles at a ratio of 1:7 cross linker to nanoparticles. Hydrolysis and condensation reactions of the silicon alkoxides (cross-linkers) were controlled by adjusting the water to alkoxide molar ratio and the solution pH as previously described by Mutlu et al. 2013. Different cross-linker solutions were prepared to create the different gels: one cross-linker solution containing Teos-Teos gel (hydrophilic gel) and other solutions with varying molar ratios of the different alkoxides: Teos and either Ptos, Vtos and Mtos at a molar ratio of 1:1—to create 50% phenyl gel, 50% vinyl gel and 50% methyl gel. A range of solutions with increasing Ptos percentage: 10-75%, was further prepared. Next, the silica nanoparticles solution, at a concentration of 400 g/L, was adjusted to pH 7 by adding 1M hydrochloric acid. The silica gels were then prepared by mixing 1.75 mL of the silica nanoparticles with 0.25 mL E. coli cells (1 g/mL) suspended in phosphate buffered saline (PBS) at pH 7.4, (or just PBS when no cells were required) and 0.25 mL of the different hydrolyzed cross-linker solutions. The solutions were left to gel for 1.5 h, resulting in silica gel plugs formed at the bottom of 20 mL scintillation vials with a volume of 2.25 mL, a 25 mm diameter and approximately 60% water content.

### Material Characterization:

Specific surface area measurements: Specific surface area was measured on dried powdered gel samples using a TriStar 3020 Surface Area and Porosity Analyzer. Gel samples were prepared as described above and then gradually dehydrated in a series of ethanol washes (50, 70, 80, 95 and 100% ethanol). The ethanol was then evaporated off the samples by keeping them overnight in the hood. Prior to analysis samples were degassed at 150° C. with N₂ gas to purge excess water and contaminants from the gel surface. Adsorption data were collected using N₂ gas as an adsorbent to obtain isotherm data at 11 different relative pressure points (Pₒ/Pₘₐₓ=0.3) under cryogenic temperatures (77° K). Surface area values (m²/g) were then calculated using isotherm data according to the Brunauer-Emmett-Teller (BET) method.

Scanning electron microscopy (SEM) measurements: Gel samples with E. coli were prepared as described above and pipeted on to a small aluminum slide. The slides were then dipped in 2.5% gluteraldehyde for 3 h and then gradually dehydrated in a series of ethanol washes (50, 70, 80, 95 and 100% EtOH). The ethanol was then evaporated off the samples by keeping them overnight in the hood. Finally, the dried gels mounted on the slides were placed on a SEM carrier and sputter-coated with a thin layer of gold-palladium. SEM images were taken by a Hitachi S4700 machine.

Confocal microscopy measurements: Samples were prepared on glass slides by depositing 300 μL of silica gel and 1 μg/mL Nile Red. Gels with cells were prepared by encapsulating E. coli expressing green fluorescent protein (GFP). All measurements were carried out using a Nikon A1si confocal system equipped with a point-scan head, 5 standard PMT detectors and a 32-channel PMT spectral detector. The system is mounted on a Nikon Ti2000E inverted fluorescence microscope with DIC optics. Nile red fluorescence was measured in the 600-650 nm range with an excitation wavelength of 561 nm. GFP fluorescence was measured in the 500-550 nm range with an excitation wavelength of 488 nm. The data for each fluorescent material were captured independently and filters were used to eliminate any overlap in the emission spectra (GFP: 500-550 nm, Nile.Red: 570-620 nm) NIS Elements imaging software was used to control acquisition and analyze the images (including particle distribution calculations). All images were taken under the same conditions and the Look Up Tables (LUT's) were adjusted to deliver comparable intensities.

In addition, A Molecular Devices SpectraMax M5 plate reader was used to quantitate the fluorescence intensity and λₘₐₓ of Nile Red in the silica gel samples (Molecular Devices, LLC., Sunnyvale, Calif., USA). Three hundred μL of Teos and 10-75% phenyl gel samples containing 1 μg/mL Nile Red were prepared in a clear-bottom 96-well plate with black sides. Samples were excited at 561 nm, with a cutoff filter at 590 nm, and emission was read from 600-700 nm. Samples were performed in triplicate.

Adsorption of atrazine to phenyl silica gels: All adsorption experiment were conducted similarly: Three mL solutions of s-triazines were added to the different gel plug in 20 mL scintillation bottles. The samples were left to shake overnight, the supernatant was then filtered through a 0.2 m Teflon filter and analyzed by HPLC. The adsorption of atrazine as a function of phenyl alkoxide content (0-75%) was done with a 50 μM atrazine solution. Adsorption isotherms of atrazine, ametryn and hydroxyatrazine were done at a concentration range of 10-100 μM. All s-triazines were analyzed using a Hewlett-Packard HP 1090 Liquid Chromatograph (HPLC) system equipped with a photodiode array detector. HPLC separations were performed using an analytical C₁₈ reverse-phase Agilent column eluted isocratically with a H₂O/MeOH solvent ratio of 35%/65% and a flow rate of 1 mL/min. Material eluting from the column were detected by ultraviolet spectroscopy with the detector set at a fixed wavelength of 220 nm.

The resulting plots from the adsorption isotherms were fitted to the Freundlich equation (Eq. 1), which relates the concentration of solute adsorbed on the surface (Y axis: C_{ads} (mmol/kg)) to the concentration remaining in the solution (X axis: C_{eq} (mg/L)).

Freundlich equation: C_{ads}=k_{f}*C_{eq}ⁿ, k_{d}=k_{f}*C_{eq}⁽ⁿ⁻¹⁾ Eq. 1

k_{f} and n are the Freundlich constants for a given adsorbate and adsorbent at a particular temperature. Adsorption coefficients for the molecules (k_{d}) were then calculated at equal concentration for all compounds (10 μM).

Measurement of adsorption kinetics of atrazine to the intact and granulated gels: Time dependent adsorption of atrazine to three gels (Teos, 50% phenyl and 75%) was measured using parallel samples for each time point and every time point was determined by duplicate samples. Supernatant was collected from the scintillation vials that contained 10 mL atrazine solution at an initial concentration of 10 μM. The supernatant sample was then passed through a 0.2 μm Teflon filter and analyzed by HPLC (UV), as described above, to determine the amount remaining in solution and to calculate the amount adsorbed on the gel. To eliminate the role of diffusion through the gel in the observed effect, gels were dried, granulated, and tested as described below. The Teos, 50% phenyl and 75% phenyl were prepared as described previously and left to dry in the hood for three days. The dried gels were then granulated into a powder using a mortar and pestle. The granulated gels were then resuspended in a 10 mL solution of 10 μM atrazine. The supernatant obtained after centrifugation at 14,000 RPM for 1 min was taken at different time points and analyzed by HPLC (UV).

Atrazine chlorohydrolase activity assays: An atrazine solution (3 mL) was added to the gel plugs (Teos, and 10-75% phenyl gels) with encapsulated biodegrading bacteria for 20 min. The solution was then drawn off from the gel, filtered through a 0.2 μm Teflon filter and analyzed by HPLC (UV).

Semi-continuous adsorption and biodegradation experiment: A 10 μM atrazine solution (3 mL) was added onto Teos or 75% phenyl gel plugs containing bacteria for repeated applications to simulate a water treatment system in which materials are sequentially exposed to contaminated water. Following 20 min applications, the water was separated from the gel, filtered and analyzed by HPLC (UV). Following separation, a fresh solution containing atrazine was immediately added to the gel for another 20 min. This procedure was repeated 6 times to reach pseudo steady-state adsorption and degradation kinetics to best simulate the conditions in a flow through reactor.

### Results and Discussion:

Choosing a silica gel precursor: The current study focuses on increasing hydrophobicity of a silica bacterial encapsulation matrix in order to enhance both adsorption and biodegradation kinetics of hydrophobic compounds. To achieve this four different gels were initially compared in terms of adsorption affinity for a model hydrophobic pollutant, atrazine. A hydrophilic, non-adsorbent, TeOs based gel was chosen as a baseline for comparison and was prepared as previously described by Mutlu et. al 2013, and three novel gels with varying hydrophobic properties were prepared by mixing the Teos alkoxide with alkoxides containing one functional hydrophobic group; either methyl, vinyl, or phenyl. Preliminary adsorption isotherms of atrazine to the different gels were constructed (FIG. 21). The affinity of atrazine to the gels increased as a function of the size and hydrophobicity of the functional group (non-functionalized<methyl<vinyl<phenyl) reaching over 90% removal of atrazine from the supernatant over 24 h by the 1:1 Teos-phenyl gel. Consequently the phenyl silica gel was chosen for further study and characterization.

Characterization of silica gels with hydrophobic patches: The silica gel that was studied contains a phenyl group in place of one of the ethoxy groups of Teos, and thus would maintain covalently attached benzene rings in the gel matrix. This phenyl silica gel with varying content of phenyl alkoxide was characterized using SEM, confocal microscopy, hydrophobic adsorption of Nile red, and surface area measurements (FIG. 21).

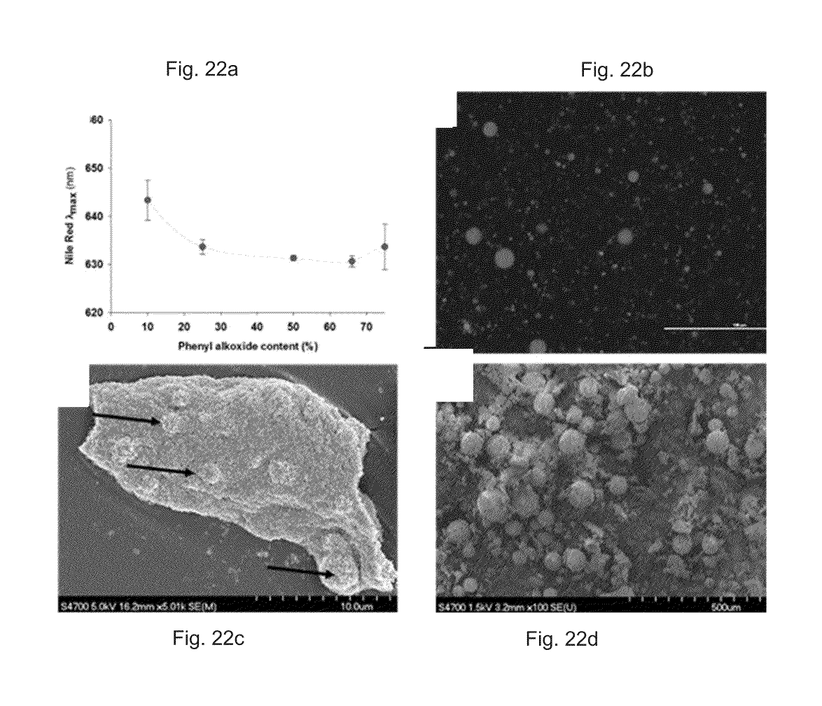

Nile red was used as a probe for hydrophobicity based on its well-known property of displaying fluorescence only in non-polar environments.³⁹⁻⁴¹ Teos and phenyl alkoxide precursors were mixed in varying concentrations to achieve 10%-75% phenyl alkoxide content and doped with Nile red. It should be noted that above 75% phenyl alkoxide content silica gels exhibited significant phase separation and consistent silica-gels were difficult to attain. Collecting the overall fluorescence signal from the gels in a microtiter well-plate reader showed a blue shift of the a, emission wavelength from 643 nm at 10% phenyl content to 630 nm at 50%-75% phenyl content indicating a change in the structure and hydrophobicity of the phenyl groups in the silica (FIG. 22a). Furthermore, a 23-fold increase in fluorescence intensity at 640 nm emission wavelength for the 50% phenyl gel as compared to a Teos gel control was measured. The shift in maximum fluorescence of Nile red as well as increase in intensity suggests that the Nile Red was partitioning into areas rich in phenyl groups. Confocal fluorescence microscopy conformed this, with images of the gels displaying the formation of distinct, nearly spherical Nile red fluorescent patches (FIG. 22b). The spheres increased in size, fluorescent intensity and amount as a function of phenyl content added. With 25% phenyl alkoxide content the spheres were in the range of 0.1-2 μm whereas for the 75% phenyl alkoxide content spheres with diameters two order of magnitude higher were measured. The SEM images presented in FIGS. 22c and 22d confirmed the formation of the spherical aggregates and also exhibited the change in formation with the increase of phenyl alkoxide content.

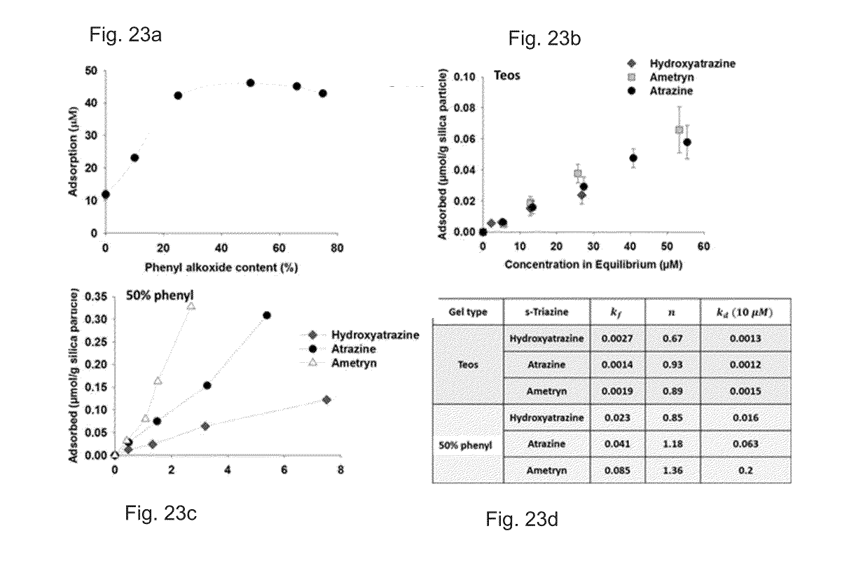

Adsorption characteristics of the phenyl gel: Adsorption of the chosen model pollutant to gels with increasing phenyl content (10-75%) was measured (FIG. 23a). Atrazine removal increased up to 50% and then plateaued, probably due to a decrease in surface area of the growing spheres which translates to available binding sites. Nevertheless, atrazine adsorption to the 50% phenyl gel (FIG. 23c was 6-folds higher than to the Teos gel (FIG. 23b), reaching over 90% removal from an aqueous solution in 24 h.

To further understand the nature of the adsorption affinity of hydrophobic compounds to the phenyl silica gel, adsorption isotherms of three s-triazine compounds with significantly different k_{ow} values to the Teos and 50% phenyl gels were constructed. The resulting adsorption plots of ametryn, atrazine and hydroxyatrazine, were fitted to the Freundlich equation to extract comparable binding coefficients (log k_{d}). The hydrophilic Teos gel showed both poor adsorptive behavior and little selectivity between the three compounds as shown in FIG. 23b and 23d. In contrast, the adsorption of the compounds to the 50% phenyl gel increased in correlation with the log k_{ow} of each compound in the order of hydroxyatrazine (k_{d}=0.016)<atrazine (k_{d}=0.063)<ametryn (k_{d}=0.2) (FIGS. 22c and 22d). These data suggest that the adsorption of the compounds is governed by the chemical affinity to the hydrophobic patches. In addition, it should be noted that the affinity of the substrate (atrazine) to the matrix is four times higher than that of the product (hydroxyatrazine). Consequently, we would expect new atrazine molecules diffusing into the gel to out-compete the product, hydroxyatrazine, which in turn can improve adsorption capacity by regenerating the adsorbent via the biodegradation reaction.

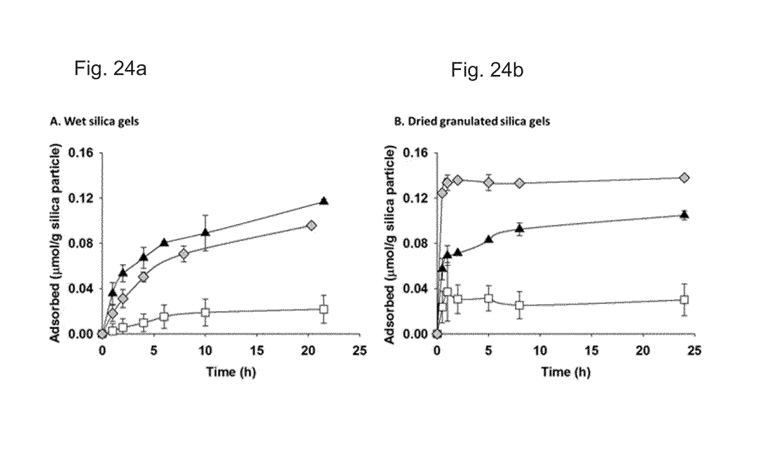

Adsorption affinity and kinetics were further characterized as a function of the phenyl content in the silica gel. In FIGS. 24a and 24b, the adsorption kinetics of atrazine to three gels Teos, 50%, and 75% phenyl gels is shown. In agreement with the adsorption isotherms, the adsorption rate also increased up to 50% phenyl gel, but a further increase in the phenyl content did not enhance the kinetics and a slight decrease in removal rates and capacity is observed. Drying and grinding the gel to decrease the diffusional barrier and expose more hydrophobic patches resulted in an observable increase in adsorption rate and capacity. In this case the kinetics and overall removal capacity of the 75% phenyl gel was higher than the 50% phenyl silica gel which strengthens our notion that the adsorption maximum at 50% is a result of the change in available reactive surface area.

Atrazine degradation by phenyl-silica gels: To assess bio-activity, Teos and 10-75% phenyl silica gels with encapsulated AtzA expressing E. coli were incubated with a 10 μM atrazine solution. Degradation rates of the silica gels with increasing phenyl content were measured by following the disappearance of atrazine and the formation of the hydroxyatrazine product by HPLC (FIG. 25). Atrazine degradation rates decreased at low phenyl content (up to 25%) and then an increase as a function of phenyl alkoxide was noticeable, reaching a threefold enhancement of rates at 75% phenyl alkoxide content.

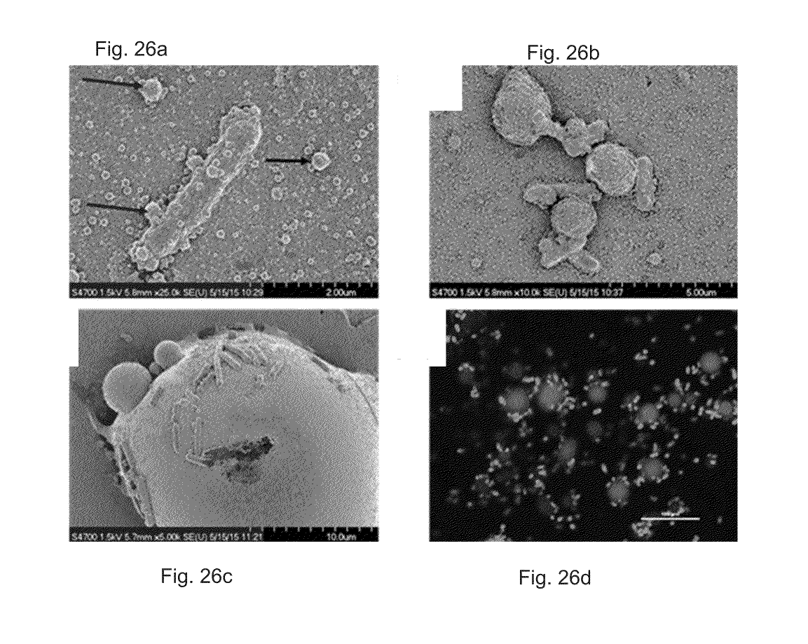

Surface area measurements and the adsorption kinetics argue against enhanced diffusivity in the phenyl gels, therefore the increased biodegradation observed is likely due to the surface and/or chemical properties of the gels and its effect on the encapsulated bacteria. Microscopy was performed subsequently to further analyze the material. FIG. 26a to 26d shows SEM images of a 25%, 50% and 75% phenyl-silica mixture with bacterial cells and the confocal fluorescent images of 75% phenyl silica gel containing E. coli expressing GFP. The SEM and confocal images reveal that bacteria could be found adhered on the hydrophobic phenyl-particles, this presented an interesting arrangement, where the substrate and the bacteria metabolizing it adhere to the same area. The images also suggest an explanation for the trends observed in the activity assays; at low phenyl content the hydrophobic aggregates are small and sparsely dispersed which present little opportunity for cell adhesion during gel formation. This consequently causes sequestration of atrazine but not its release to the cells (FIG. 26a). At higher phenyl content (above 50%) the hydrophobic aggregates are larger and more abundant which present more reactive areas for the cells to attach to. The phenyl aggregates can then act as a conduit, facilitating atrazine diffusion to the adjacent cells (FIG. 26b and 26c). The wider frame confocal image containing phenyl aggregates doped with Nile red and encapsulated bacteria expressing GFP, also show that the bacteria adhere to the phenyl functionalized particles (FIG. 26d). Adhesion to hydrophobic surfaces such as polystyrene has been reported for a large number of bacteria and is known to be governed by Van der Waals and electrostatic interactions.⁴⁴⁻⁴⁶ E. coli have a slight negative charge as well as hydrophobic moieties in the outer membrane; which would explain the selectivity towards the particles with reduced negative charge and increased hydrophobicity.

Semi-continuous adsorption and biodegradation: To gain some idea of the potential applicability of the effects observed here, a semi-continuous adsorption and biodegradation experiment was conducted. Note that we have previously observed that silica encapsulated non-viable E. coli cells expressing AtzA degrade atrazine without loss of activity for at least 4 months. Teos and 75% phenyl gels with encapsulated AtzA expressing E. coli were incubated with a 10 μM atrazine solution for 6 consecutive 20 min time periods and compared for adsorption and degradation efficiency. These experiments were conducted on three different days, each time in three replicates.

Consistent with the previous adsorption and activity experiments, the rate of atrazine removal and the rate of hydroxyatrazine formation was each approximately 3 fold higher for the 75% phenyl gel compared to the Teos gel (FIGS. 27a and 27b). Furthermore, the removal of atrazine by the Teos gel reached a pseudo steady-state after 2 incubation periods and the amount of hydroxyatrazine formed was equal to the amount of atrazine removed, showing no new net adsorption. By contrast, the 75% phenyl gel reached adsorption saturation only after 6 washes and still the amount of hydroxyatrazine formed was only slightly lower than the amount of atrazine removed.

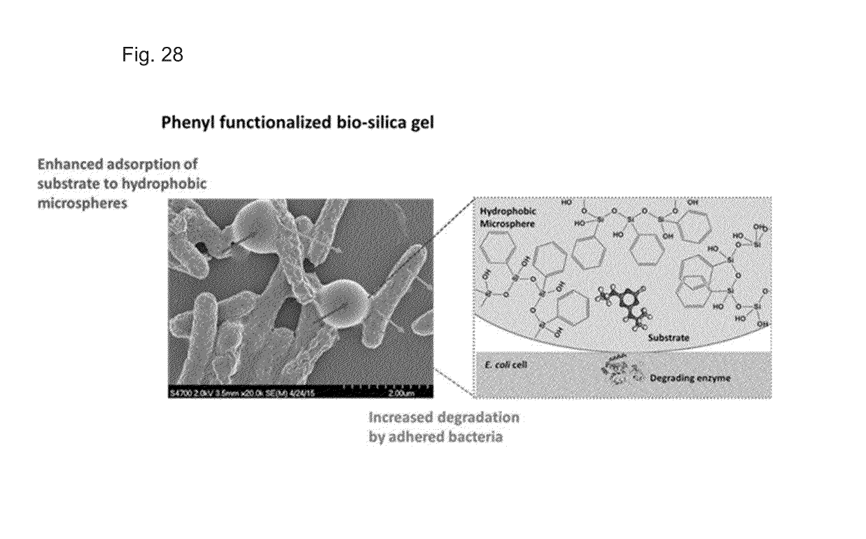

FIG. 28 shows a SEM image of bacteria absorbed on the silica matrix and a schematic depiction of the overall material.

Overall, these results exhibit a combined system whereby the adsorption and degradation processes are both enhanced, unlike conventional bio-GAC systems or behavior in natural environments. Further studies will focus on elucidating sub-micron features of the hydrophobic patches, the molecular placement of groups within the spheres, and the properties of bacteria that can impede or enhance transfer of chemicals from the hydrophobic spheres to enzymes within the bacteria. Further delineation of the structure and mechanism of bio-reactive hydrophobic silica gels could potentially widen the field of environmentally applicable materials that can be tuned to effectively combine adsorption and biodegradation.

### 5. Parathion Degradation Enhanced at Low Concentrations with 75% Phenyl-Containing Gel

Parathion, an insecticide, has the following structure:

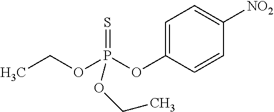 [SMILES: CCOP(=S)(OCC)Oc1ccc([N+](=O)[O-])cc1]

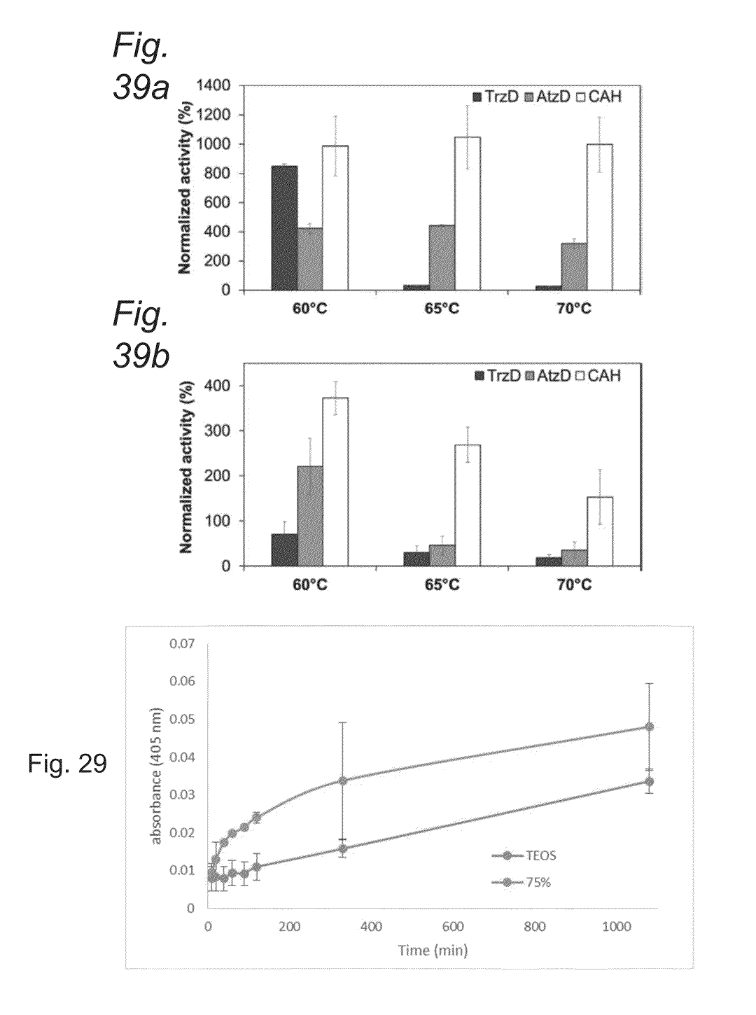

The degradation of parathion was compared in a 75% phenyl-containing (25% TEOS) silica matrix and a 100% TEOS matrix, both of which included the same bacteria. FIG. 29 shows the absorbance (which increases as p-nitrophenol, a degradant of parathion, is formed) versus time for 0.7 mM parathion with 0.02 g/mL cells.

### 6. Multilayer Construction

Silica gel encapsulation of bacteria is a promising method with a wide range of engineering applications, including biosynthesis, biocatalysis and bioremediation. One major advantage of silica encapsulation is the tunability of the gel matrix, in terms of its porosity, pore size and surface energy. Thus, a silica gel matrix can be tailored for a specific application, to maximize activity of the encapsulated bacteria and enhance transport of the necessary substrates in the gel. However, this approach yields a highly specialized gel which is optimized for a specific bacteria and bio-transformation. A more complex application where multiple cells, substrates or phases are present cannot be addressed with this approach.

In this example, a silica gel with multiple layers of varying characteristics was developed. The layers can differ in their surface energy (hydrophilicity/hydrophobicity), microstructure (porosity/pore size), or any other property. In this example, the surface energy was adjusted between the two by incorporating organically modified (with hydrophobic side groups) silicon alkoxides to the gel composition. This enabled enhanced transport of different substrates in different layers, with possible extension to multi-phase fluids. The microstructure could also be modified by using different silica precursors, varying sizes of silica nanoparticles, changing pH during silica gel synthesis and/or incorporating polymers (e.g. polyethylene glycol) to induce phase separation. An application for these macro-pores could be to serve as channels in the matrix to reduce the diffusion length for the substrates. Furthermore, a multi-layered structure also allows different microbial strains to be encapsulated in different layers. This is beneficial since these layers can be optimized for those strains and the cells are still in close proximity.

Bioprinting (i.e. 3D printing of biologically active materials) is an exciting new area where biomaterials or compositions containing biomaterials are precisely deposited layer-by-layer to build organs or viable tissues. A similar method can be utilized in this kind of application, where the cells are replaced by bio-transforming bacteria and the matrix is a silica hydrogel. These layers can be printed using conventional (e.g. spin coating) or non-conventional (e.g. ink-jet printing) methods.

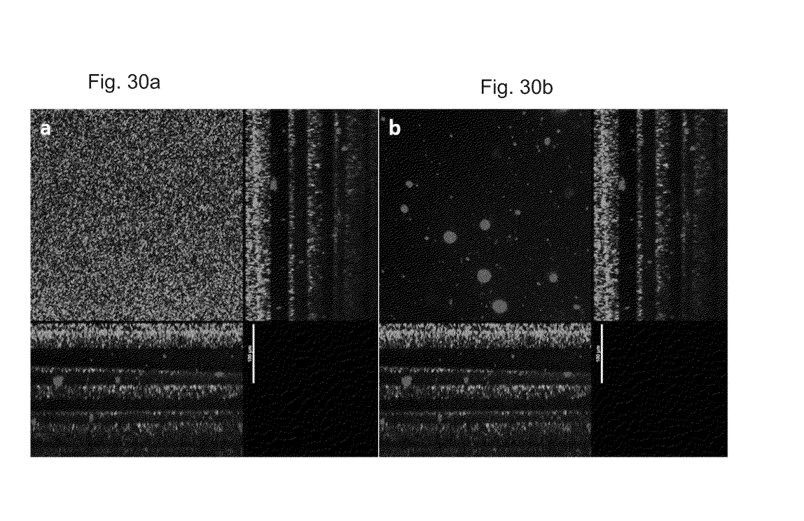

In this example, a multi-layered gel has been synthesized using spin coating, where one layer is a hydrophilic gel with encapsulated green fluorescent protein expressing bacteria and the other is a hydrophobic gel with Nile red stain (FIGS. 30a and 30b). Note that while the spin coating method allows spatial arrangement only in one axis (thickness of layers), ink-jet printing will allow synthesis of complex 3D structures of microbial-silica gels.

### 7. Silica Gel Matrix Including Amine Functionalized Groups

Silica gel matrices including an amine cross linker were studied in order to determine if their inclusion could have an effect on the diffusion of a target component through the cell membrane of the biomaterial. Escherichia coli strains expressing an oxygenase, an azo reductase, glutathione S-transferase, and simple hydrolase were used to show the effect of the diffusion issue on different classes of enzymes. The relative rates of encapsulated versus free cells varied considerably; in some cases, encapsulated cells were almost comparable to the free cells but in most cases the activity decreased 2-4 folds. The observed difference in activity between encapsulated and free cells was mainly a function of substrate properties (MW, hydrophobicity and solubility) which limit diffusion to the silica matrix and through the cell membrane. Silica gel matrices incorporating a precursor with a propyl amine group were developed and tested; the presence of amine functionalized groups resulted in a significant increase in activity for the gels and free cells but not with the free enzyme. Further experiments revealed that the increase in activity is due to mild damage to the cell membrane, allowing easier access to the enzyme. This method of bacteria encapsulation can offer a convenient, effective and inexpensive means of increasing bio-activity of cells for diverse substrates acted on by all known classes of enzymes.

In this example, an encapsulation method applicable to a wide range of enzymes and substrates was developed. Hydrolases, which are mostly the focus of bio-catalytic enzymes/cells for encapsulation, comprise only one of the six major Enzyme Commission classes that also include: oxido-reductases; transferases; lyases; isomerases; and ligases. These reaction types carry out the diverse bio-transformations required for life and also provide a broad potential for industrial bio-catalysis beyond hydrolytic reactions. Very little work has been done to use whole cells entrapped in silica gels to carry out non-hydrolytic reaction pathways that would require the regeneration of cofactors that participate in the reactions. Going beyond hydrolytic reactions often requires the use of cofactors and co-substrates that become highly expensive in vitro. Thus, it would be desirable to maintain whole cells in a bio-catalytically active state for long periods of time as has been observed for cells catalyzing hydrolysis reactions while encapsulated within silica gels.

In this example, the membrane permeability issue was addressed by treating the gels with an amine functionalized precursor. Diffusional issues were investigated by comparing reactions between encapsulated cells in several types of amine functionalized gels and free cells. Activity was demonstrated with five recombinant enzymes from four different Enzyme Commission classes to stress the wide applicability of this method. This technology will enable development of cheap, sustainable and competitive encapsulation matrices applicable for a wide range of enzymes and substrates.

### Materials and Methods

### Materials:

The cross-linker precursors used in the silica-gel preparation; Tetraethyl-orthosilicate (TeOs) and Amino-propyl trethoxysilane (APTES) were purchased from Sigma-Aldrich (Sigma-Aldrich Corp. St. Louis, Mo., USA). The silica nanoparticles (Nex-sil 125-40, 80 nm diameter) were purchased from Nyacol (Nyacol Nano Technologies Inc., Ashland, Mass., USA). Technical grade atrazine was provided by Syngenta (Syngenta Crop Protection, NC, USA).Methy Red, 1-chloro dinitro benzene (CDNB), dihydroxy phenyl acetic acid (DHPA) and all other reagents used for buffers, HPLC solvents etc. were purchased from Sigma-Aldrich.

### Bacterial Growth Conditions:

Five types of enzymes were expressed in E. Coli DH5 alpha: AtzA (atrazine chlorohydrolase), Azo reductase, Homoprotocatechuate 2,3-dioxygenase and Glutathione-S transferase.

AtzA: Atrazine degrading bacteria (degrades atrazine to hydroxyl atrazine) (Mutlu, B. R.; Yeom, S.; Tong, H.-W.; Wackett, L. P.; Aksan, A., Silicon alkoxide cross-linked silica nanoparticle gels for encapsulation of bacterial biocatalysts. Journal of Materials Chemistry A 2013, 1, (36), 11051-11060).

Cyanuric acid hydrolase: Cyanuric acid degrading bacteria (degrades cyanuric acid to biuret (Cho S, Shi K, Seffernick J L, Dodge A G, Wackett L P, et al. (2014) Cyanuric Acid Hydrolase from Azorhizobium caulinodans ORS 571: Crystal Structure and Insights into a New Class of Ser-Lys Dyad Proteins. PLoS ONE 9(6)).

Azo reductase: Degrades azo-dyes by cleavage of the amide bonds—needs NADPH electrons (Chen et. al, Microbiology (2005), 151, 1433-1441).

Homoprotocatechuate 2,3-dioxygenase: Uses oxygen to degrade (cleaves) hydroxylated phenyl ring (Groce et al. Biochemistry 2004, 43, 15141-15153).

Glutathione-s transferase: Catalyzes the conjugation of the reduced form of glutathione (GSH) to the CDNB substrate for the purpose of detoxification.

### Silica Gel Preparation:

Hydrolysis and condensation reactions of the TeOs based silicon alkoxide (cross-linkers) were controlled by adjusting the water to alkoxide molar ratio and the solution pH as previously described by Mutlu et al. 2013 (Mutlu, B. R.; Yeom, S.; Tong, H.-W.; Wackett, L. P.; Aksan, A., Silicon alkoxide cross-linked silica nanoparticle gels for encapsulation of bacterial biocatalysts. Journal of Materials Chemistry A 2013, 1, (36), 11051-11060). The alkoxide to water molar ratio was set to 1:5.3:0.0013 (alkoxide:water:HCl), which according to previous literature results in a fully-hydrolyzed silicon alkoxide solution with a slow condensation rate (Mutlu, B. R.; Yeom, S.; Tong, H.-W.; Wackett, L. P.; Aksan, A., Silicon alkoxide cross-linked silica nanoparticle gels for encapsulation of bacterial biocatalysts. Journal of Materials Chemistry A 2013, 1, (36), 11051-11060). APTES cross-linker was hydrolyzed by adding 1.5 mL of initial alkoxide to 8.5 mL of DI water with 40 μL of HCL. The preparation included adjusting the pH of the silica nanoparticles solution to neutral pH by adding 1M hydrochloric acid (concentration: 400 g/L), and mixing 1.75 mL of the neutralized silica nanoparticles with 0.25 mL E. coli cells suspended in phosphate buffer saline (PBS) pH 7.4 (or just PBS when no cells were required) and 0.25 mL of the hydrolyzed cross-linker solution (either TeOs or APTES). Three different gels were prepared, the first containing TeOs alkoxide only (hydrophilic gel), the second was prepared as a TeOs based gel with added hydrolyzed APTES overlayed for the last 30 min of gelation time (the solutions were left to gel for 1.5 h), and the third gel was prepared using hydrolyzed APTES alkoxide only. The resulting gels were in plug form (volume: 2.25 mL with approximately 60% water content). All adsorption and activity experiments were carried out with these gel plugs in 20 mL scintillation vials.

### Activity Assays:

For each enzyme different activity assays were used (see Table 3). Detection of the substrate/product was done by UV-Vis and HPLC (depending on the compound).

TABLE 3
Diverse Enzyme Reactions, Substrate Properties and Activity Assay

| | | | Kow of | solubility | |
|---|---|---|---|---|---|
| Enzyme | Substrate | MW | substrate | (mg/L) | Assay |
| Ring | 3,4 | 167.14 | 0.47 | 236000 | 3,4-Dihydroxyphenylacetate 2,3- |
| cleavage | Dihydroxypheny | | | | dioxygenase was followed by |
| dioxygenase | acetate | | | | the formation of 5- |
| (recombinant | | | | | carboxymethyl-2- |
| in E. coli) | | | | | hydroxymuconic acid |
| | | | | | semialdehyde (CHMSA) at 380 nm. |
| | | | | | The rate of formation of the |
| | | | | | product was calculated from ε = |
| | | | | | 35,500 for CHMSA at pH 7.8. |
| Hydrolayse | atrazine | 215.7 | 2.6 | 34.7 | The degradation of atrazine and |
| (recombinant | | | | | formation of hydroxyatrazine |
| in E. coli) | | | | | was followed by HPLC. HPLC |
| | | | | | Analysis was done using a |
| | | | | | Hewlett-Packard HP 1090 |
| | | | | | Liquid Chromatograph system |
| | | | | | equipped with a photodiode |
| | | | | | array detector. The detection |
| | | | | | method used an analytical C18 |
| | | | | | reverse-phase Agilent column at |
| | | | | | a wavelength of 220 nm, a |
| | | | | | H2O/MeOH solvent ratio of |
| | | | | | 35%/65% and a flow rate of 1.0 mL/min. |
| Glutathione | 1-chloro2,4- | 202.55 | 2.06 | 394.07 | The formation of chloro- |
| S-transferase | dinitrobenzene | | | | dinitrobenzene-GS complex was |
| (recombinant | | | | | measured spectroscopically at |
| in E. coli) | | | | | 340 nm. The rate of formation of |
| | | | | | the product was calculated from |
| | | | | | ε = 9600. |
| Azoreductase | methyl red | 291.28 | ? | 800,000 | The cleavage of the azo bond |
| (recombinant | sodium salt | | | | was followed by the decrease in |
| in E. coli) | | | | | the absorbance of the substrate, |
| | | | | | methyl red at 430 nm |
| Cyanuric | Cyanuric acid | 129.07 | 1.95 | 2700 | Formation of biuret by HPLC, |
| acid | | | | | using normal phase amino |
| hydrolase | | | | | column at 212 nm. |

### Activity Assays for Encapsulated Whole Cells:

A 3 mL solution of the chosen substrate (atrazine/DHPA/CDNB or Methyl red) was added to the three gel plugs (TeOs/TeOs+APTES and APTES) with the respective encapsulated bacteria. The solution was then separated from the gel, filtered and analyzed either by UV-Vis or by HPLC.

### Activity Assays for Free Cells:

A solution of free cells was mixed with the cross-linker alone (without the silica nanoparticles) to evaluate the effect of the cross-linker on free cells (no gel formation). The ratios were the same as the gel activity ratios: 0.25 mL of 0.1 g/mL E. Coli suspended cells and 0.25 mL of cross-linker solution were added to 1.75 mL solution of PBS. A 3 mL solution of the chosen substrate (atrazine/DHPA/CDNB or Methyl red) was added in for the respective enzyme times and then the cells were separated by centrifuge in an Eppendorf tube (14,000 RPM for 1 min) and the supernatant was filtered and analyzed either by UV-Vis or HPLC.

### Activity Assay for Free Enzymes:

A solution of free cells was lysed using a French pressure cell press. The remaining solution containing the free enzyme was used for the assay. The ratios were the same as the gel and free cell activity ratios: 0.25 mL of 0.1 g/mL E. Coli suspended enzyme and 0.25 mL of cross-linker solution were added to 1.75 mL solution of PBS. A 3 mL solution of the chosen substrate (atrazine/DHPA/CDNB or Methyl red) was added in for the respective enzyme times and then the cells were separated by centrifuge in an Eppendorf tube (14,000 RPM for 1 min) and the supernatant was filtered and analyzed either by UV-Vis or HPLC.

### Multiple Activity Assays for Homoprotocatechuate 2,3-Dioxygenase:

Whole cells expressing homoprotocatechuate 2,3-dioxygenase were encapsulated in the three gel types (TeOs, TeOs+APTES and APTES alone). A 3 mL solution of 150 μM DHPA was added to the three gel plugs (TeOs/TeOs+APTES and APTES) for 10 min. This activity assay was carried out on the gels four times over a period of two days. The remaining solution of each assay was removed after analysis and a fresh solution was introduced when the next assay began. The product formation, CHMSA, was followed by UV-Vis absorbance at 380 nm.

### Azo Reductase Activity Assay in the Presence of NADPH:

APTES-gels with encapsulated azo-reductase expressing bacteria were prepared. A 3 mL solution of 70 μM Methyl Red was added to the gels in the presence of NADPH in excess. The supernatant was monitored at 430 for the disappearance of methyl red over 20 h. A control was set up with no NADPH.

### Confocal Measurements:

### Effect of Encapsulation on Cell Membrane:

The effect of encapsulation on the cell membrane was visualized by confocal spectroscopy using propedium iodide (PI). PI fluoresces when in contact with cellular DNA (meaning the membrane is permeable). The TeOs and Amine functionalized gels were prepared with E. coli DH5α and PI dye. The samples were prepared on glass slides by depositing 300 μL of silica gel doped with 1 μg/PI. All measurements were carried out using a Nikon A1si confocal system equipped with a point-scan head, 5 standard PMT detectors and a 32-channel PMT spectral detector. The system is mounted on a Nikon Ti2000E inverted fluorescence microscope with DIC optics. PI was measured at an excitation wavelength of 561 nm and an emission range of 600-650 nm, NIS Elements imaging software was used to control acquisition and analyze the images.

### Results:

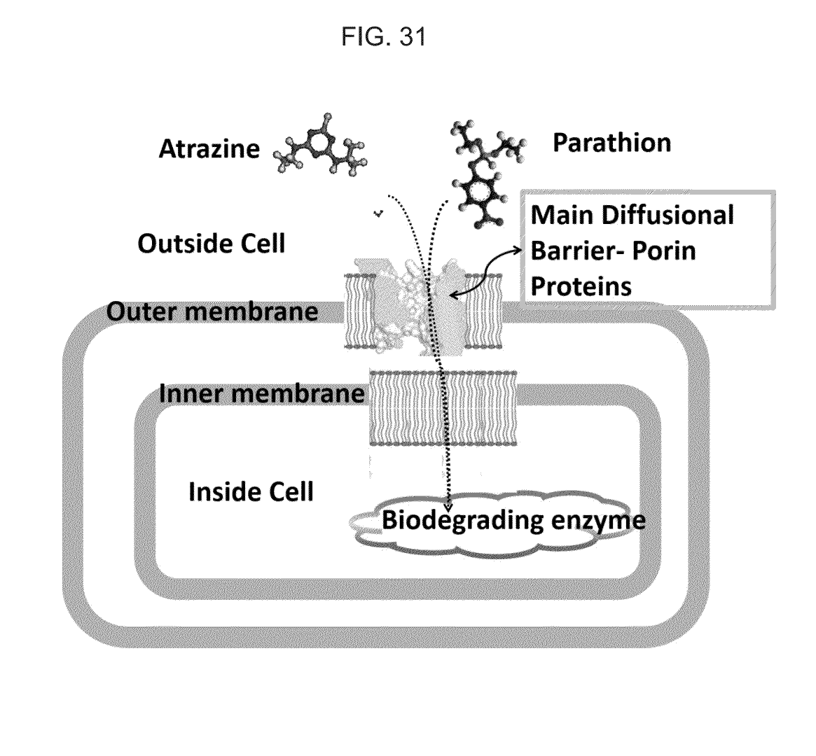

Initially the diffusion barriers were assessed by evaluating the rate ratio between free cells and cells encapsulated in the baseline gel previously prepared by Mutlu et al. 2013 (FIG. 31 and Table 3). The degradation rates were generally higher for free cells compared with the encapsulated cells (between 2-10 times higher). It is thought, but not relied upon, that this is due to diffusional barriers through the encapsulation matrix and the membrane. This hypothesis is strengthened by the correlation between the substrate properties and the difference of free cells and the TeOs gel degradation ratios; slower rates (and higher ratios) are observed for the more hydrophobic compounds in the gels (higher kow, MW and lower solubility).

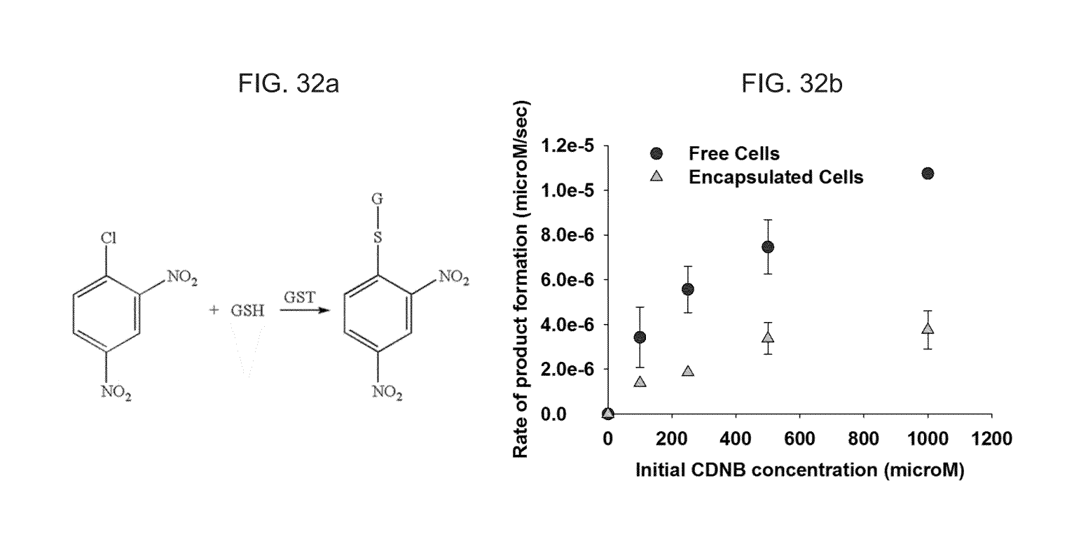

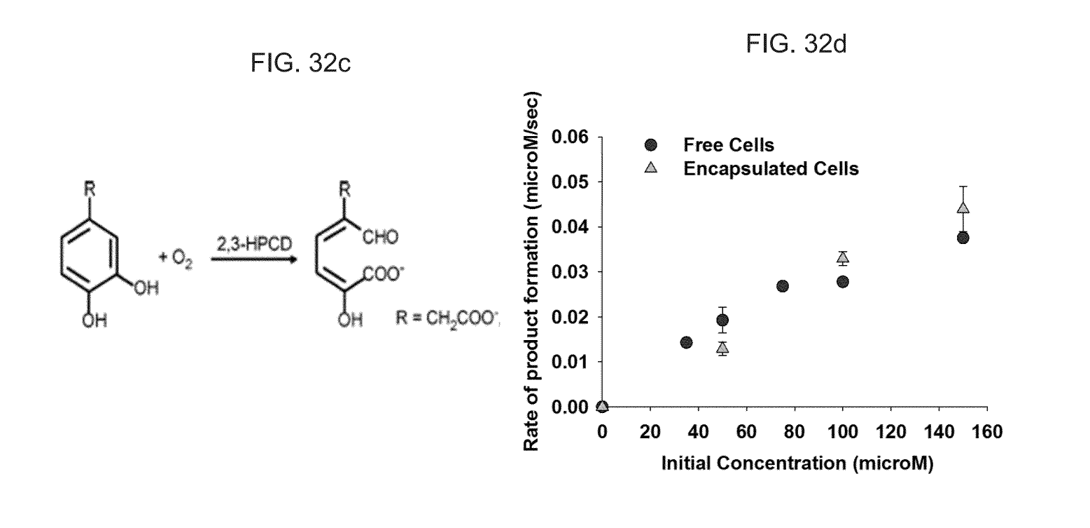

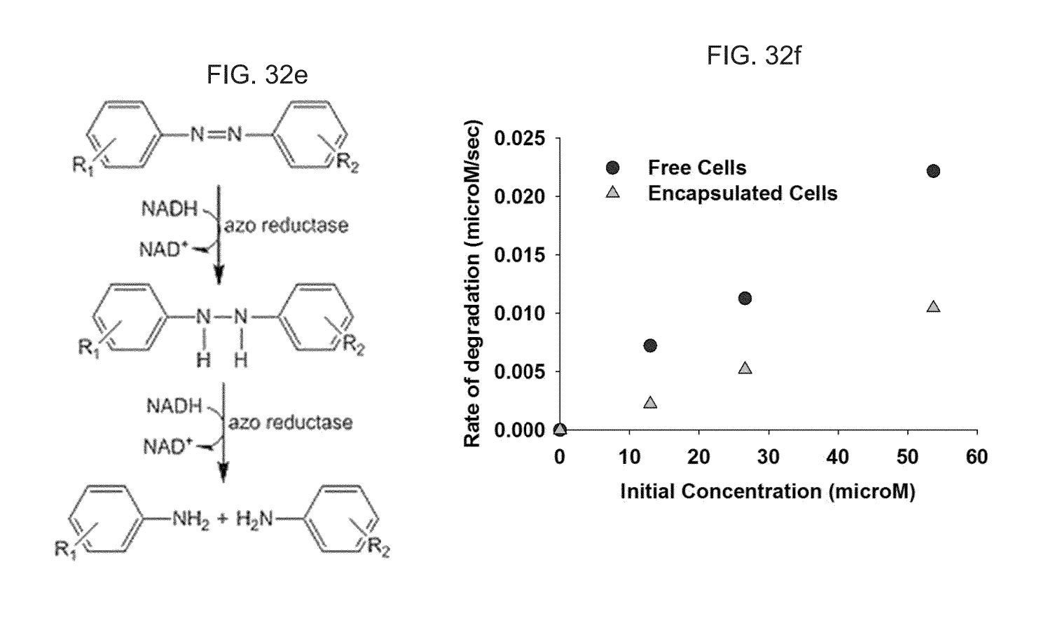

FIG. 32 shows glutothione transferase (FIGS. 32a and 22b), homoprotocatechuate 2,3-dioxygenase (FIGS. 32c and 32d), and Azo reductase (FIGS. 32e and 32f) enzymatic reactions of whole cells encapsulated in TeOs based gels and free in solution. It should be noted that ATZA free cells and encapsulated cells have been previously published by Mutlu et al. 2013

Table 4 shows the ratio of the free cell rates versus the encapsulated cell rates for the diverse reactions.

TABLE 4
Ratio of free cell rates vs encapsulated rates for the diverse reactions

| Enzyme and substrate | Ratio (free cells/gel) |
|---|---|
| Dioxygenase: 3,4 Dihydroxypheny | 1-2.5 |
| acetate (100-200 microM) | |
| CYA hydrolase | 1-2 |
| Azo reductase: Methyl red sodium salt (50-150 | 1.8-3.7 |
| microM) | |
| Glutothion transferase: 1chloro2,4 | ~3.8 |
| dinitrobenzene (100 microM) | |
| AtzA: Atrazine (25 microM) | ~8.75 |

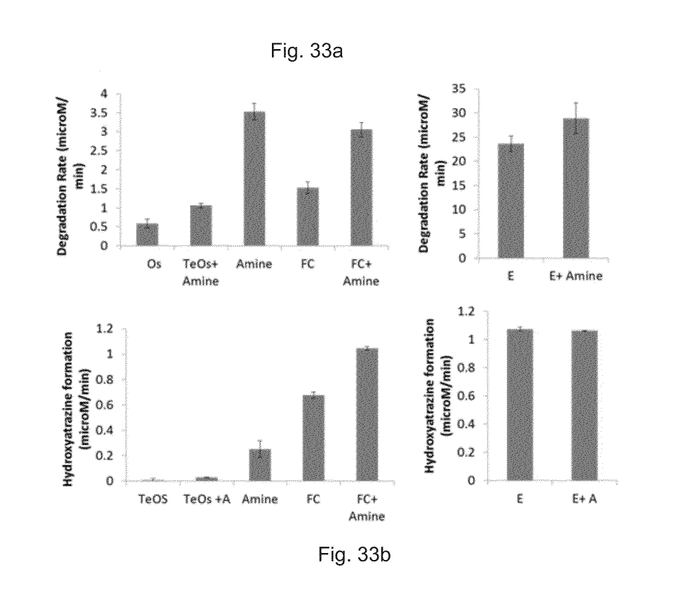

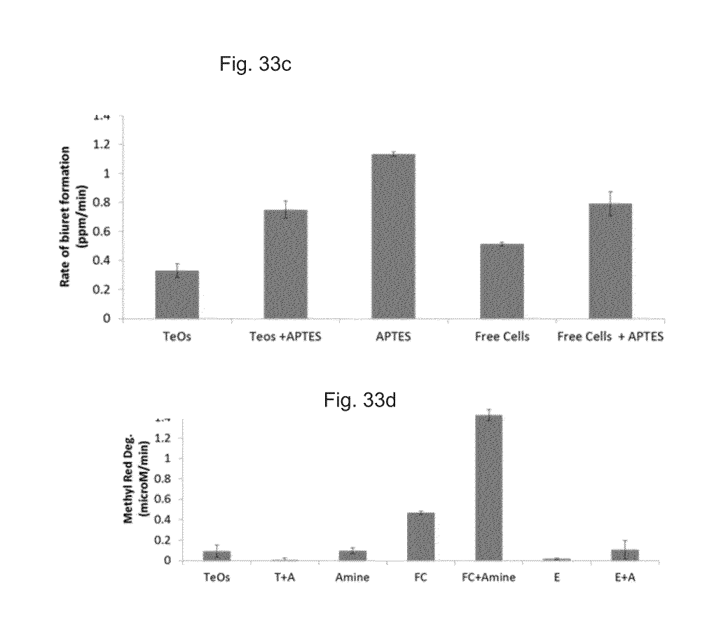

To overcome the diffusional barrier, amine functionalized gels were developed. Studies have indicated that amine groups can create membrane damage and facilitate diffusion to the target enzyme (Milovic et al, BIOTECHNOLOGY AND BIOENGINEERING, VOL. 90, NO. 6, JUN. 20, 2005, Hong et al. Bioconjugate Chem. 2004, 15, 774-782 and Hong et al. Bioconjugate Chem. 2006, 17, 728-734). Three gels were developed, the first containing TeOs alkoxide only (TeOs in FIGS. 33a to 33d), the second was prepared as a TeOs based gel with added hydrolyzed APTES overlaid (TeOs+Amine in FIGS. 33a to 33d) and the third gel was prepared using hydrolyzed APTES alkoxide only (Amine in FIGS. 33a to 33d). Activity of the different whole cells was tested and compared to free cells (FC in FIGS. 33a to 33d), free cell plus amine (FC+Amine in FIGS. 33a to 33d), free enzyme in solution (E in FIGS. 33a to 33d), and free enzyme plus amine in solution (E+Amine). FIGS. 33a to 33d show the degradation rate in the various gels for dioxygenase (FIG. 33a), AtzA (FIG. 33 b), cyanuric acid hydrolase (FIG. 33c), and Azo Reductase (FIG. 33d).

The results demonstrate the increase of activity in the different silica gels as a function of amine functionalization for the first three reactions. Reaction rates increase 2-3 fold depending on the enzyme when comparing the TeOs base line gel with the TeOs with the APTES overlaid and a much higher increase is noticeable with the APTES gels (no TeOs). This is because with the APTES overlaid the cells are pre-encapsulated in the TeOs gel and are protected from the amine group that is added after the gelation has begun. The APTES gels allow longer and facile contact with the amine group, resulting in more membrane damage and consequently easier transport.

In the case of Azo reducatase, a more complex behavior is observed. It is thought, but not relied upon that this is because the reaction needs nicotinamide adenine dinucleotide phosphate (NADPH) as a cofactor and permeabilizing the cell membrane results in significant loss of the co-factor molecule and therefore of activity. Since NADPH is a relatively large molecule (MW=744), this permeability hypothesis was tested by evaluating whether the molecule can actually enter the cell after the membrane was exposed to APTES.

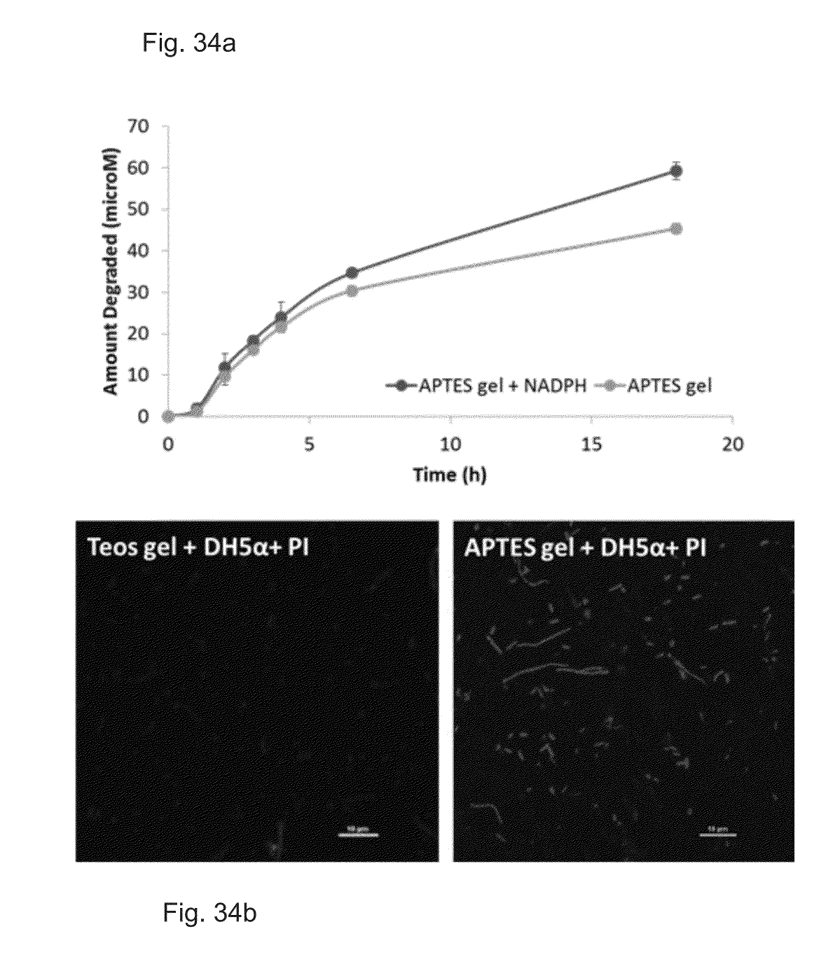

In FIG. 34a, the activity of whole cells expressing azo-reductase encapsulated in the silica-APTES matrix over time (Methyl red sodium salt degradation) in the presence of externally added NADPH is shown. Initially the rates are comparable because NADPH is still present in the cell microenvironment; however, at longer times, without excess of NADPH, activity decreases whereas in the presence of externally added NADPH activity is higher. This suggests that at least a partial amount of NADPH enters the cells and is available for the reaction.

To visualize the permeability, confocal images of E. coli stained with PI and encapsulated in Teos and APTES gels were captured (FIG. 34b). PI fluoresces only when in contact with the cellular DNA, therefore it is commonly used to distinguish live intact cells from permeablized cells. The images indicate higher fluorescence in the APTES gels, which strengthens the hypothesis that the cells are being punctured.

### Testing Cell Leakage:

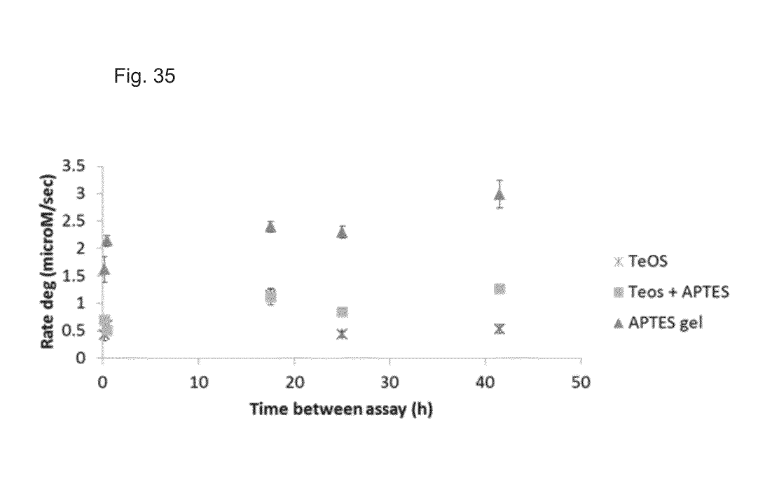

Since the cell membrane was probably damaged by the presence of amine groups, we wanted to evaluate whether the activity decreased due to enzyme leakage, when the gels were applied multiple times. Whole cells expressing homoprotocatechuate 2,3-dioxygenase were encapsulated in the three types of gels and activity was tested four times over the course of two days. Each time the supernatant was thrown out and a fresh substrate was added to the gel. The results displayed in FIG. 35 show no reduction of activity in any of the gels, suggesting that enzyme does not leak out and activity is sustained over time and after multiple applications.

To summarize, this method for developing silica gel matrices to encapsulate bacteria for bio-catalysis improves on prior materials by overcoming a major diffusional barrier and enhancing bio-catalytic activity. This technology will enable formulation of a matrix that is catalytically comparable or even higher than free cells but at the same time maintaining the enzyme protected and active for long periods of time.

### 6. Bacterial Cyanuric Acid Hydrolase for Water Treatment

In the present study, Escherichia coli cells expressing three different cyanuric acid hydrolases were each studied for their ability to degrade cyanuric acid under conditions most likely to be used in a flowthrough system. The most well studied cyanuric acid hydrolases, TrzD from Acidovorax avenae subsp. citrulli, AtzD from Pseudomonas sp. strain ADP, and CAH from Moorella thermoacetica ATCC 39073, were chosen. The optimum enzyme for these purposes was found to be the cyanuric acid hydrolase from M. thermoacetica. A water-recycling, flowthrough system was constructed and shown to be effective in removing 10,000 μM cyanuric acid, a concentration well above that encountered in real-world disinfection processes.

### Materials and Methods

Bacterial strains and culture conditions. E. coli strains were grown at 37° C. in LB medium with vigorous aeration. Three recombinant strains expressing the cyanuric acid hydrolases AtzD, TrzD, and CAH (Moorella thermoacetica cyanuric acid hydrolase, from open reading frame Moth_2120) were used (see Table below). E. coli BL21(DE3)(pET28b+CAH) was induced by adding 0.5 mM isopropyl-β-D-thiogalactopyranoside (IPTG) to the culture when an optical density at 600 nm (OD600) of 0.5 was reached. The induced cells were grown overnight at 37° C.

When required, antibiotics were added at 100 μg ampicillin ml⁻¹ and 50 μg kanamycin ml⁻¹.

TABLE 5
Strain, plasmid, or primer used in this study

| Strain, | Relevant markers/ | Reference or |
|---|---|---|
| plasmid, or primer | characteristics or sequence (5′→3′)^{a} | source |
| Strains | | |
| DH5α | Δ(lacZYA-argF)U169(φ80lacZΔM15) | Lab stock |
| BL21 (DE3) | F⁻ ompT hsdSᵤ (rᵤ mᵤ)gal dcm (DE3) | Life Technologies |
| CAH | Strain DH5α harboring pUCMod CAH; Amp^{r} | This study |
| AtzD | Strain DH5α harboring pUCMod AtzD; Amp^{r} | This study |
| TrzD | Strain DH5α harboring pUCMod TrzD; Amp^{r} | This study |
| CAH-induced | Strain BL21 (DE3) harboring pET28b+ | This study |
| | CAH; Km^{r} | |
| Plasmids | | |
| pUCMod | rep (pMB1) bla (Amp^{r}), constitutive lac | 17 |
| | promoter | |
| pUCMod CAH | pUCMod carrying the M. thermoacetica | This study |
| | cyanuric acid hydrolase gene | |
| pUCMod AtzD | pUCMod carrying Pseudomonas sp. strain | This study |
| | ADP atzD gene | |
| pUCMod TrzD | pUCMod carrying the Acidovorax avenae | This study |
| | subsp. citrulli trzD gene | |
| pET28b+ CAH | pET28b+ carrying the M. thermoacetica | 16 |
| | cyanuric acid hydrolase gene | |
| Primers | | |
| CAH-F | GAA TTC AGG AGG ATT ACA AAA TGC AAA AAG | |
| | TCT TTC GTA TCC CAA CAG | |
| CAH-R | ATT ACC ATG GCT ACA CCC TGG CAA TAA CAG | |
| | CAA TTG GG | |
| Atz-F | ATT GAA TTC AGG AGG ATTA CAA AAT GTA TCA | |
| | CAT CGA CGT TTT CCG AAT CCC TTG CCA C | |
| Atz-R | ATT TAA TGC GGC CGC TTA AGC GCG GGC AAT | |
| | GAC | |
| TrzD-F | ATT GAA TTC AGG AGG ATT ACA AAA TGC AAG | |
| | CGC AAG TTT TTC GAG TTC C | |
| TrzD-R | ATT TAA TGC GGC CGC TTA AGC TGT GCG CGC | |
| | GAT AAC | |

^{a}Amp^{r} and Km^{r}, resistance to ampicillin and kanamycin. In the primer sequences, underlined letters restriction enzyme recognition indicate a Shine-Dalgarno sequence.

Cloning procedures and plasmid construction. To construct E. coli strains containing cyanuric acid hydrolase, pET28b+::Moth 2120, pET28b+::atzD, and pET28b+::trzD were utilized as the PCR templates. The gene from Moorella thermoacetica ATCC 39073 was amplified from pET28b+::Moth 2120 with the primers CAH-F and CAH-R. The fragment of the CAH gene was cloned into the EcoRI and NcoI cloning sites of the StrataClone PCR cloning vector (Agilent Technologies, Inc.). The resulting plasmid was digested with the same restriction enzymes, and the fragment released from the StrataClone plasmid was ligated into pUCMod, yielding pUCMod CAH (Table 5). The plasmid was introduced into MAX Efficiency E. coli DH5 competent cells (Life Technologies). A CAH-induced strain was constructed by introducing the vector pET28b+::Moth 2120 into One Shot BL21(DE3) chemically competent E. coli (Life Technologies), thereby generating E. coli BL21(DE3)(pET28b+CAH) (Table 5). The full lengths of atzD and trzD were amplified from pET28b+::atzD and pET28b+::trzD, respectively, via PCR with the primers AtzD-F, AtzD-R, TrzD-F, and TrzD-R. The fragments were then cloned into the EcoRI and NotI cloning sites of the pUCMod vector, yielding pUCMod atzD and pUCMod trzD (Table 5). The plasmids were introduced into E. coli DH5 by electroporation. E. coli DH5 competent cells were prepared by washing cells harvested at the exponential phase (OD600 of 0.5) with distilled water and a 10% (vol/vol) glycerol solution.

Encapsulation. Silica-encapsulated cells were prepared in either molds, 20-ml glass scintillation vials, or 4-ml glass tubes. Reagent-grade tetraethyl orthosilicate (TEOS) was purchased from Sigma-Aldrich. Nex-Sil 125-40 colloidal silica nanoparticles (SNP) were purchased from Nyacol Nano Technologies Inc. TEOS was hydrolyzed by stirring at a 1:5.3:0.0013 molar ratio of TEOS to water to HCl for 2 h(18). The pH of the NexSil 125-40 SNP was adjusted to pH 7.0 by adding 1 μM hydrochloric acid. After pH adjustment, an appropriate amount of cells suspended in phosphate-buffered saline (PBS) (pH 7.4) was added to the SNP solution to obtain a cell loading density of 0.125 g of wet cell mass/ml of the final gel. Gelation was started by adding hydrolyzed TEOS to the mixture of SNP and cells at a 7:1 SNP/TEOS volume ratio at room temperature.

Cyanuric acid hydrolase activity assays. Cells for assay were grown overnight, harvested by centrifugation, and resuspended into PBS at a density of 0.3 g wet cell mass per ml. The reaction was initiated by either adding 0.03 g of resuspended cells or exposing 2 ml of silica gel containing encapsulated cells to 3 ml of 10 mM cyanuric acid solution in 0.1 M potassium phosphate buffer (pH 7.0). Samples were incubated with shaking at 120 rpm and collected after 30 min of incubation. The samples were filtered through a 0.2-m-pore-size polytetrafluoroethylene (PTFE) syringe filter. No detectable enzyme activity was found to be released from the silica gels during the course of the experiments. To assay for the enzymatic conversion of cyanuric acid to biuret and then to ammonia, the biuret hydrolase from Rhizobium leguminosarum bv. viciae strain 3841 was purified as described previously and coupled with the cyanuric acid hydrolase activity. One mole of biuret is trans-formed to one mole each of allophonate and ammonia by biuret hydro-lase, and ammonia was quantified using the hypochlorite-phenol reaction (scheme below). Enzyme assays were conducted for 30 min of incubation with an excess of biuret hydrolase. Positive controls with known concentrations of biuret were conducted in parallel to ensure that all of the biuret was converted to ammonia. The scheme for the coupled assay for cyanuric acid hydrolase activity using biuret hydrolase and measuring stoichiometric formation of ammonia via the hypochlorite-phenol reaction is seen below and is described above.

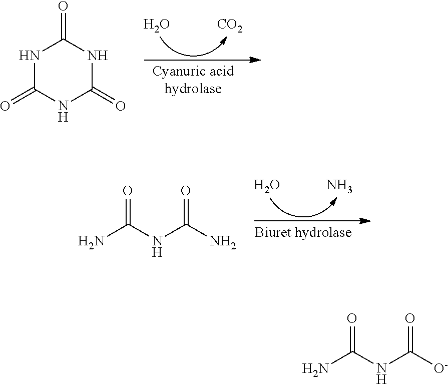

Cyanuric acid hydrolase activity was also determined by measuring cyanuric acid disappearance with the addition of 20 mM melamine in 0.1 M phosphate buffer to form a 1:1 melamine-cyanuric acid complex that can be quantified by its turbidity by apparent absorbance (light scattering) at 600 nm. A standard curve of cyanuric acid showed this method to be linear within the range used in these experiments. All incubations and assays were conducted at 22° C. All of the enzymes are significantly active at this temperature, and their relative reaction rates with heat treatment have been compared previously.

Inactivation of hydrolase-producing E. coli cells by heat treatment. Two milliliters of encapsulated cells in a 20-ml glass scintillation vial (thickness, 3.5 mm; diameter, 24 mm) was used in these studies. For experiments with suspended cells, the cells were grown overnight and resuspended in 0.1 M phosphate buffer (pH 7.0) with a density of 0.01 g of wet cell mass/ml in a 1.7-ml microcentrifuge tube. The samples were placed in a water bath adjusted to 60° C., 65° C., or 70° C. (or left at 22° C. as a control) for 1 h and then placed on ice for 5 min. Suspended cells were pelleted by centrifugation and resuspended into the same buffer. Cyanuric acid hydrolase activity was measured as described above with a cell density of 0.001 g of wet cell mass/ml. Encapsulated cells were tested as gel plugs in the bottom of vials as described above.

Oxygen consumption. Cells were encapsulated in a 4-ml glass tube with a diameter of 6 mm as 200-1 cylindrical blocks. Measurements of oxygen consumption were conducted using a Hansatech Oxytherm sys-tem (Hansatech Instruments). Three milliliters of LB medium was pipetted into the chamber of the Oxytherm device. The chamber was sealed after the encapsulated sample was placed inside. The data were exported to a Stains for cellular membrane disruption. One microliter of each dye solution was added to 1 ml of cell suspension. Twenty millimolar pro-pidium iodide (PI) dissolved in dimethyl sulfoxide was one cell stain. The intensity of fluorescence was measured with a fluorescence spectropho-tometer (Molecular Devices SpectraMax M2) using an excitation wave-length of 535 nm and an emission wavelength of 617 nm. BacLight Green bacterial stain (Invitrogen) was prepared according to the to manufacturer's instructions. The BacLight Green bacterial stain was measured at an excitation wavelength of 480 nm and an emission wavelength of 516 nm. All measurements were corrected by subtracting the small background fluorescence observed with a phosphate buffer control. The data were exported to a computerized chart recorder (Oxygraph; Hansatech Instruments).

Cyanuric acid degradation measurement with the flowthrough system. Cells were encapsulated as hemispherical silica beads (1.0 to 1.5 mm in diameter) in molds as previously described (18). A 6.6-g quantity of the beads was placed in the bioreactors. One liter of 10,000 M cyanuric solution in 0.1 M potassium phosphate buffer (pH 7.0) was the influent solution to the bioreactors and was circulated at a flow rater of 360 ml/h. Four days after completion of the first experiment, the channels were flushed with 0.1 M phosphate buffer (pH 7.0) for 1 h, and then the treatment of a fresh 1-liter 10,000 M cyanuric acid solution commenced. Samples of 0.5 ml were collected and tested for cyanuric acid degradation using biuret hydrolase and the hypochlorite-phenol method as described above.

Pool water degradation. Pool water samples were taken from three different swimming pools in the Twin Cities area and tested for pH, hypochlorite, and cyanuric acid levels. One gram of 1-mm spherical silica beads containing CAH-induced cells was incubated in 200 ml pool water at room temperature with shaking at 120 rpm. A buffered water-20 mM sodium phosphate-137 mM NaCl-2.7 mM KCl solution at pH 7.6 was used as a positive control. Cyanuric acid concentrations were determined by measuring the formation of the melamine-cyanuric acid complex as described above. Hypochlorite concentrations were measured by the N,N-dimethyl-p-phenylenediamine (DPD) colorimetric method (20). A DPD solution (3.9 mM) was prepared by dissolving 16 mg of DPD in 25 ml water (pH 2.0). A freshly opened 5.25% sodium hypochlorite solution was used to prepare a standard curve. The oxidation of DPD by hypochlorite was monitored at 550 nm on a Beckman DU 640 spectrophotometer (Beckman Coulter, Fullerton, Calif.).

### Results

Use of a sensitive enzyme-coupled assay for determination of cyanuric acid hydrolase activity. Previous studies assaying cyanuric acid hydrolase activity used a direct spectrophotometric measurement of substrate disappearance at 214 nm. This assay was ineffective in the present studies because even minor impurities from the cells, glassware, or tubing strongly contributed to absorbance at 214 nm. Cyanuric acid and biuret can be analyzed with high-pressure liquid chromatography, but the present study demanded a large number of assays to be conducted rapidly. In that context, it was found here to be most effective to measure cyanuric acid hydrolase activity by converting the product of the reaction, biuret, stoichiometrically to ammonia with purified biuret hydrolase. Degradation as low as 5 nmol per ml could be determined this way, and this was at least twice as sensitive as other methods. Water samples without cyanuric acid were run as a control, and ammonia leakage from cells was found to be negligible. This method was compared to directly measuring cyanuric acid disappearance in water by adding melamine and measuring the extent of the 1:1 melamine-cyanuric acid precipitant as described in Materials and Methods. Both methods gave consistent results, but the biuret hydrolase coupled assay was more sensitive and thus was used routinely herein.

Comparison of cyanuric hydrolase activities in whole cells. The three cyanuric acid hydrolases, TrzD, AtzD, and CAH, were expressed in the same E. coli cell background so that diffusion through cell membranes, expressions levels, and other interacting proteins and other cell properties would be the same. Each enzyme was expressed constitutively with the same expression system. SDS-PAGE confirmed that the expression levels of the enzymes were similar and that the majority of each respective cyanuric acid hydrolase was found in the soluble fraction of cell lysates (see FIG. S2 in the supplemental material). Our previous studies with cyanuric acid hydrolases expressed in E. coli also did not encounter problems with inclusion body formation. In an in vivo comparison of the three enzymes, TrzD showed the highest activity (FIG. 37a). This result is consistent with a previous report that TrzD showed approximately a 2-fold-higher kcat/Km than AtzD and CAH. This increase was attenuated when using cells encapsulated into silica gels (FIG. 37b), due to the diffusion limitation imposed by the silica gel matrix. This limitation also causes an order-of-magnitude difference between the activity rates of suspended and encapsulated cells (compare FIGS. 37a and 37b). The rate-limiting effect of diffusion in an encapsulated cell system can be reduced by decreasing the size of the material. Since the focus of this study was to compare the relative activities and stabilities of different cyanuric acid hydrolases in vivo when encapsulated, the diffusion properties of the gels were not optimized.

Viability and inactivation of hydrolase-producing E. coli cells by heat treatment. The use of encapsulated cells in a disinfection treatment system would be most acceptable if the cells could be rendered nonviable while retaining all or nearly all cyanuric acid hydrolase activity. It is possible to have activity in non-viable cells because the enzyme is a hydrolase, it does not require cofactors, and, in that regard, it resembles atrazine chlorohydrolase, which has been shown previously to remain fully active for over 4 months in nonviable E. coli cells. Nonviability is defined as the inability to replicate and/or when cells have disrupted membranes that allow molecules to freely diffuse in and out, as typically shown with dyes. Moreover, the equilibrium for the cyanuric acid hydrolase reaction is completely in the direction of product formation. We previously showed that the reaction is essentially irreversible due to rapid and spontaneous decarboxylation of the enzyme product, carboxybiuret, which leads to the stoichiometric formation of the stable product, biuret.

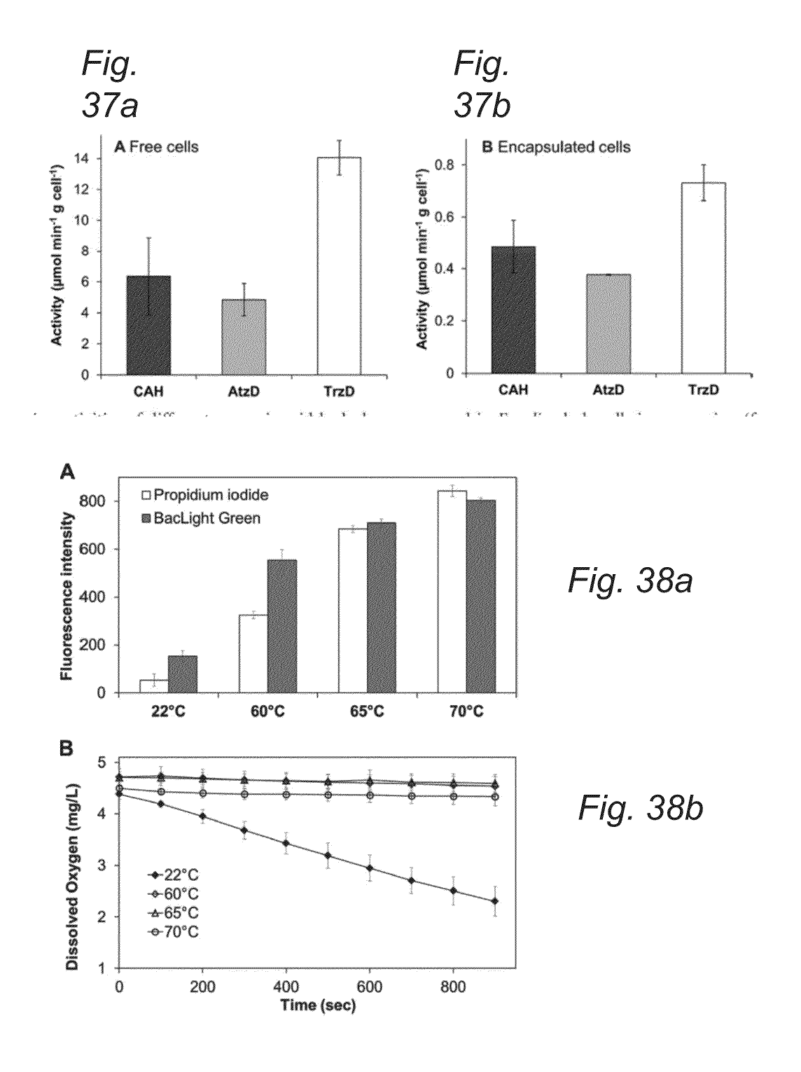

In the first experiment, E. coli cell suspensions were heated to temperatures (60° C.) known to induce 100% cell death (25). Treatment at 60° C., 65° C., and 70° C. resulted in no viable cells, which was confirmed by plating heat-treated cells on rich medium plates. We next tested cell viability/permeability using commonly accepted methods. The two fluorescent dyes propidium iodide (PI) and BacLight Green are known to give increased fluorescence when cells become nonviable or show loss of membrane integrity. Here, we treated cells at 22° C., 60° C., 65° C., and 70° C., and the fluorescence went up dramatically between 22° C. and 60° C., suggesting that the cell membranes became permeable by heat treatment (FIG. 38a). This is consistent with the plate count results showing a complete loss of E. coli cell replication ability between 22° C. and 60° C. etric formation of the stable product, biuret.

To determine the viability of encapsulated cells, the silica matrix was pulverized to a fine powder using a mortar and pestle, and the released cells were suspended in sterile PBS. Cell counts were determined based on the number of observed CFU after overnight incubation on rich medium plates. By this measure, no cell viability was observed at 60° C., 65° C., or 70° C. However, the plate count method could underestimate cell viability in silica gel because en-capsulated cells may not become fully separated from the silica matrix during the grinding process. To further investigate viability, we tested for metabolic activity by looking for oxygen consumption in the presence of a rich, oxidizable substrate mix with an Oxygraph. Encapsulated cells at room temperature consumed about half of the dissolved oxygen within 15 min, whereas encapsulated cells heated to 60° C. or higher showed no discernible oxy-gen consumption (FIG. 38b).

Cyanuric acid hydrolase activity measurements after heat treatment. In the next set of experiments, cyanuric acid hydrolase activity was determined following heat treatment at 60° C., 65° C., or 70° C. for 1 h and cooling back down to assay temperature. All of the cells showed an increase in activity following treatment at 60° C., presumably due to membrane disruption that led to greater substrate diffusion into the cell (FIG. 39a). It has been reported that heat treatment at 55° C. and above causes vesiculation and blebbing in the outer membrane of E. coli. The loss of lipopolysaccharide (LPS), which poses a significant barrier to substrate entry into cells, was also observed in other studies. However, at 65° C. and 70° C., the cyanuric acid hydrolase activity for cells express-ing TrzD dropped precipitously, whereas AtzD and CAH remained consistent (FIG. 39a). At 70° C., CAH activity remained at the same level following the 60° C. and 65° C. heat treatments. These results are consistent with in vitro studies comparing CAH to AtzD and TrzD, in which CAH was shown to be more thermally stable. Circular dichroism (CD) spectroscopy has shown that the thermal denaturation temperature of purified CAH in buffer is above 70° C.

When examining the heat stability of cyanuric acid hydrolase activities in silica-encapsulated cells, the trend was similar but the greater stability of CAH than of TrzD and AtzD was even more dramatic (FIG. 39b). The increase in the activity above 100% (the activity was normalized to that measured prior to heat treatment) was likely due to membrane damage leading to increased permeability to cyanuric acid. However, this effect at 65° C. or 70° C. was more than nullified with TrzD and AtzD by the inactivation of the enzymes. It is currently unclear why the encapsulated enzyme showed an even greater sensitivity to heat treatment than enzyme in nonencapsulated cells. However, at 65° C., the CAH activity remained more than 250% of that observed prior to heat treatment, making CAH very attractive for obtaining high cyanuric acid degradation activity while rendering bacteria nonviable.

Several approaches have been employed in whole-cell enzyme applications to render cells more permeable, for example, using organic solvents or detergents. The present data suggest that heat treatment with cells encapsulated in silica and containing a thermostable enzyme can be used to reduce substrate permeability barrier while also rendering cells nonviable, which is a desirable feature for water treatment applications.

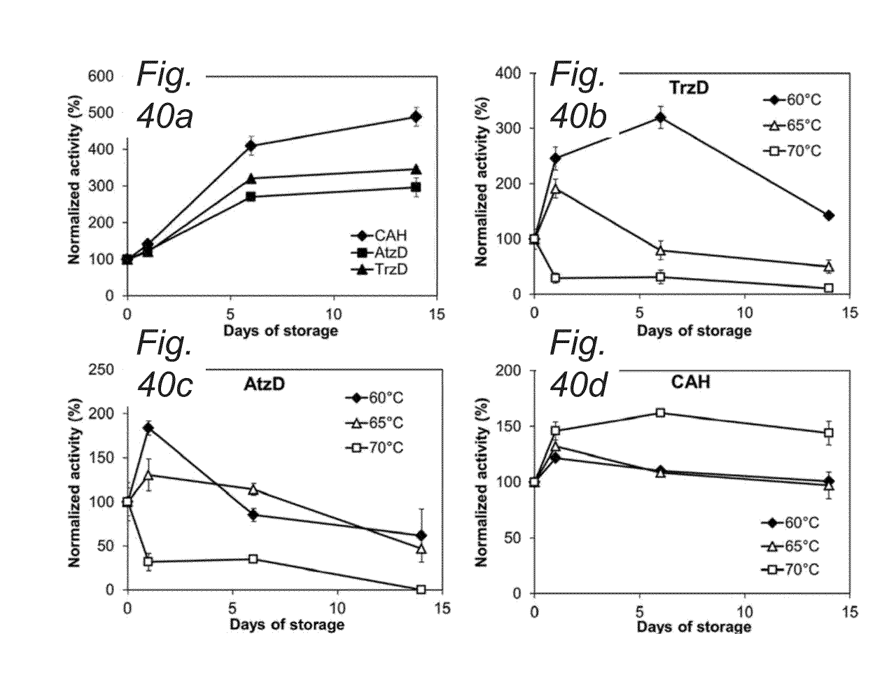

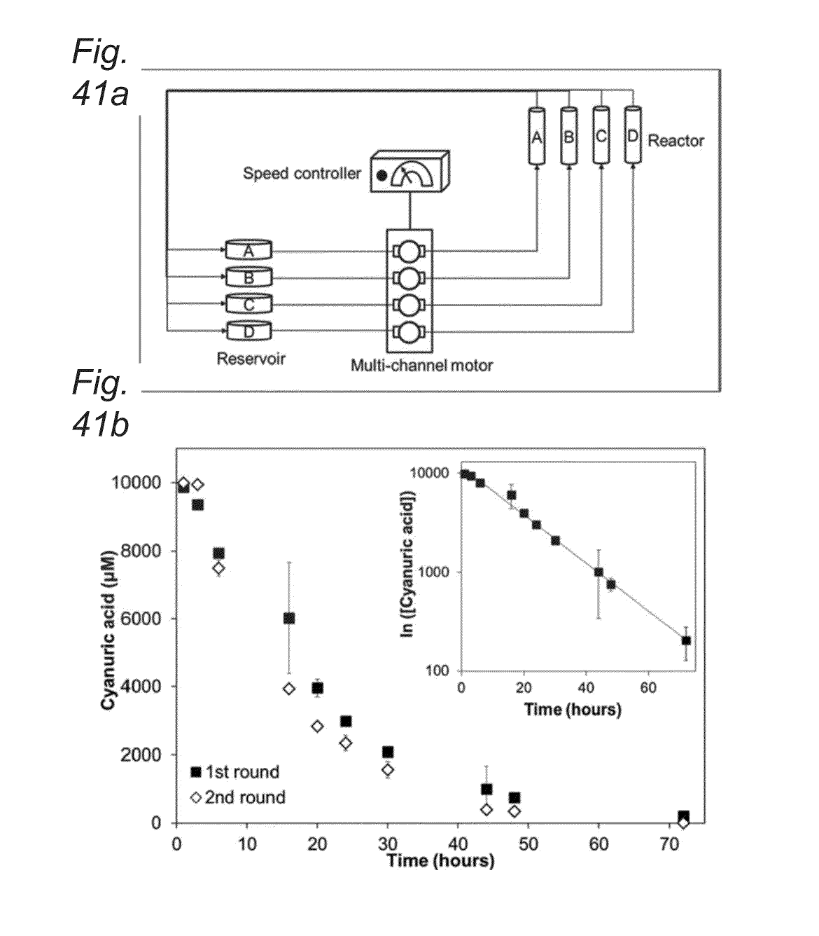

Storage stability of encapsulated cells. In order to evaluate storage stability, encapsulated cells were subjected to no heating or to heating at 60° C., 65° C. or 70° C. and then maintained at room temperature. No special treatment was done, nor were stabilizing agents added. Individual stored gels were sampled at the time points indicated in FIG. 40a to 40d and assayed for cyanuric acid hydrolase activity. When encapsulated cells were maintained at room temperature at all times, the cyanuric acid activities with AtzD, TrzD, and CAH all increased substantially, with CAH increasing the most to more than 400% of the original activity (FIG. 40a). Note that encapsulated E. coli cells expressing atrazine chlorohydrolase activity also showed an increase upon storage for 2 weeks, although it was less than 200% of the original activity, which had been attributed to a time-dependent disruption of the cell membrane allowing greater permeability of the substrate.

The effect of heat treatment on stability of cyanuric acid hydrolase activity was also investigated. With TrzD, the measured activity after heat treatment and 1 day of storage increased at 60° C. and 65° C., but the activity decreased to 20% of the level for un-treated, nonstored samples after heat treatment at 70° C. and stor-age for 1 day (FIG. 40b). Upon long-term storage (14 days), all of the heat-treated and stored samples showed a decrease in activity to at best around the level for nonheated, nonstored encapsulated TrzD cells. The results with AtzD were qualitatively similar to those with TrzD cells (FIG. 40c). After long-term storage, all samples showed less than the original levels of activity. The CAH encapsulated cells showed a much more robust response (FIG. 40d). The initial gains in activity upon storage were modest, <150% of the starting activity, but the level of activity remained quite constant thereafter. These results are significant because a product for treatment of pools and spas will likely require shelf storage for weeks and months, and the present data suggest that the formulation containing the CAH enzyme is best able to meet those criteria.

Flowthrough cyanuric acid treatment. In light of the storage and thermal stabilities of the CAH activity, E. coli expressing CAH was chosen to test for practical applications simulating a flowthrough pool water treatment system. In this experiment, silica beads with a 1.0- to 1.5-mm diameter were used. There were four packed columns, two with beads containing active CAH enzyme in vivo, one with beads alone, and one empty (FIG. 41a). Each column was treated by pumping buffer containing 10,000 M cyanuric acid through the column. The 1 liter of cyanuric acid solution was pumped at a 6-ml/min flow rate through over 24 cycles. From the control experiment with beads alone, we could deter-mine that the amount of ammonia release from cells that was unrelated to cyanuric acid hydrolase activity could account for at most 0.5% of the observed degradation. It was shown that over 7,000 M cyanuric acid was removed in 24 h, and the complete degradation of cyanuric acid was observed in 72 h (FIG. 41b). To further test the longevity of the system, after the column system had been at room temperature for a week, the identical experiment was run. FIG. 41b shows that the cyanuric acid degradation activities were virtually indistinguishable. This further indicates that the CAH enzyme is highly stable, as there was not even a slight loss of degradation activity under operating conditions and over the course of 1 week.

The observed decrease in cyanuric acid was plotted to determine if it followed first- or second-order kinetics, and the fit was much better with a first-order model. A least-square fit for the first-order linear plot of the logarithm of cyanuric acid concentration versus time yielded an r2 value of 0.99 (FIG. 41b, inset). A plot of the data assuming a second-order model yielded a much poorer fit to a straight line, with an r2 value of 0.74 (data not shown). Thus, a first-order decay of cyanuric acid is indicated over the entire range of cyanuric acid concentration, from 10,000 M to the final concentration of essentially 0 M after 72 h.

The observed first-order biodegradation of cyanuric acid with encapsulated cells can be contrasted with what would be expected with isolated purified enzyme in solution, for which the Km has been determined to be 110 M. The time course of cyanuric acid degradation by the isolated enzyme can be calculated by solving for substrate concentration as a function of time with the integrated Henri-Michaelis-Menten equation. If the enzyme were put into a 10,000 M cyanuric acid solution, the degradation of the substrate would proceed in an essentially zero-order fashion for greater than 95% of the substrate disappearance, in contrast to what was observed in FIG. 41b. This large difference in the kinetics for the isolated enzyme and the encapsulated whole cells suggests either that the in vivo enzyme has a Km orders of magnitude higher than that of the in vitro enzyme as tested or that the in vivo cyanuric acid concentration is very low, such that first-order kinetics hold. The latter explanation is more plausible given that the cell membranes, the silica matrix surrounding the cells, and the concentration gradient likely formed throughout the length of the column can all lead to a low effective concentration of cyanuric acid in the cell cytoplasm where the enzyme is ex-pressed. Several observations are consistent with this hypothesis.

First, the activity of the cells increased up to 5-fold over the first 2 weeks of storage. Similar observations were made with E. coli cells expressing another hydrolytic cytoplasmic enzyme, atrazine chlorohydrolase, and that was shown to correlate with changes in the membranes that led to increased entry of the substrate into the cell. In other studies, the silica matrix was directly tested for chemical diffusion and shown to impose rate limitations over lengths of more than 0.1 mm, and the current beads ranged from 1.0 to 1.5 mm in diameter, consistent with a significant diffusional barrier imposed by the matrix.

While the diffusional barriers of cell membranes and silica matrix likely impose lower degradation rates than for purified enzymes, a commercial treatment system for a swimming pool will need to meet certain requirements of cost that make the use of purified enzyme prohibitive. Moreover, the silica matrix can be rendered more porous to enhance diffusion, but this will cause a corresponding decrease in mechanical strength causing beads to disintegrate in flow systems, as has been previously described. The silica beads used in the flowthrough experiments maintained mechanical integrity throughout the two tests.

Test of encapsulated E. coli expressing CAH with swimming pool waters. There might be additional chemicals present in actual swimming pool waters that would affect the in vivo CAH activity, and so this was tested directly. Recently opened swimming pools from the Twin Cities metropolitan area were sampled in mid-June 2015. Be-cause it was early in the season, the addition of chlorinated isocyanuric acids was relatively low, and so we spiked the waters with additional cyanuric acid to make it up to a level that would require treatment (>100 ppm or 775 M). Otherwise the waters were not modified and were of similar pH (7.3 to 7.4) but contained different levels of hypochlorite. The silica-encapsulated cells with CAH were gently shaken in each of the three swimming pool waters, along with a water control (with no hypochlorite).

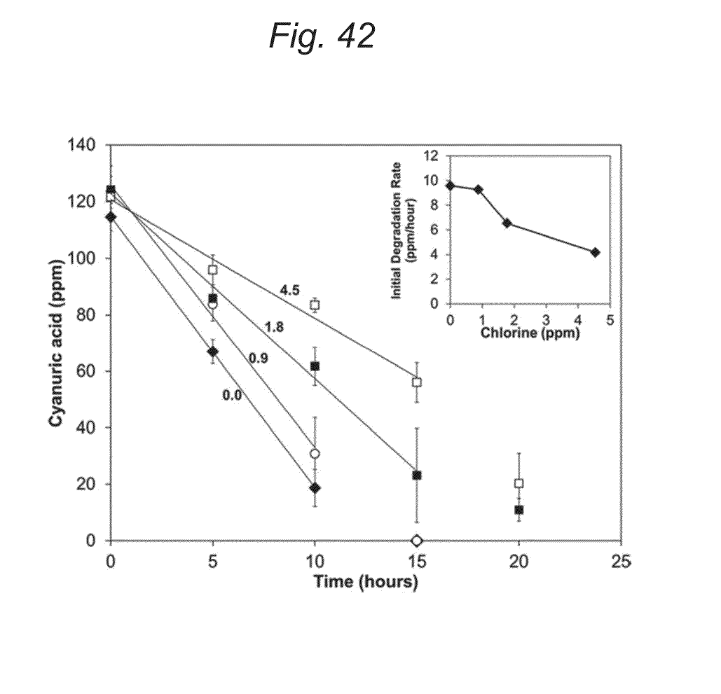

The pool waters all showed substantial reductions in cyanuric acid over a 20-h period, but there was a difference observed as a function of the hypochlorite concentration (FIG. 42). Without hypochlorite, or at a concentration of 0.9 ppm (17 M), the initial cyanuric acid was at undetectable levels at 15 h. At 1.8 ppm (34 M) and 4.5 ppm (85 M) hypochlorite, there was still residual cyanuric acid at 10 (194 M) and 20 (388 M) ppm, respectively, after 20 h. The initial rates of cyanuric acid degradation were plotted as a function of hypochlorite concentration, and this showed a significant effect of chlorine, decreasing the rate approximately 50% at the highest level of hypochlorite (4.5 ppm) compared to the no-hypochlorite control. Swimming pools generally contain <5 ppm (97 M) hypochlorite to avoid irritation to swimmers, so the levels tested here go to near the highest accepted levels and show that the encapsulated E. coli cells expressing the Moorella cyanuric acid hydrolase still function at that level, albeit at a diminished rate.

In general, the use of silica-encapsulated cells containing the Moorella cyanuric acid hydrolase (CAH) offers a good trade-off, with significant rates, activity maintenance during storage and use, heat stability allowing cell killing with heat, and overall me-chanical stability in a flowthrough system. In a swimming pool, it is generally desirable to diminish cyanuric acid concentrations from levels of 120 ppm (930 M) to approximately 40 ppm (310 M). In swimming pool waters, the presence of hypochlorite was observed to diminish rates significantly. Further studies are war-ranted to investigate the effects of hypochlorite, develop mitigation strategies, and deliver cost-effective microbial enzymatic systems for swimming pool treatment.

In a first embodiment, a composition is provided that includes a first silica-matrix encapsulated biomaterial, the first silica-matrix encapsulated biomaterial including a first silica matrix and a first biomaterial; and a second silica-matrix encapsulated biomaterial, the second silica-matrix encapsulated biomaterial including a second silica matrix and a second biomaterial, wherein the first silica-matrix encapsulated biomaterial has at least one property that is different than that of the second silica-matrix encapsulated biomaterial, and wherein the first silica-matrix encapsulated biomaterial forms a first layer and the second silica-matrix encapsulated biomaterial forms a second layer, and the first layer is positioned adjacent the second layer.

In a second embodiment, the composition according to the first embodiment, wherein the at least one property can be chosen from the porosity, the permeability, the surface charge, the surface functionality, the average pore size, the surface energy, and the chemical composition.

In a third embodiment, the composition according to the first embodiment, wherein the at least one property is surface energy.

In a fourth embodiment, the composition according to the third embodiment, wherein the first silica-matrix encapsulated biomaterial is more hydrophobic than the second silica-matrix encapsulated biomaterial.

In a fifth embodiment, the composition according to the first embodiment, wherein the at least one property is porosity.

In a sixth embodiment, the composition according to the fifth embodiment, wherein the first silica-matrix encapsulated biomaterial is more porous than the second silica silica-matrix encapsulated biomaterial.

In a seventh embodiment, the composition according to the first embodiment, wherein the at least one property is average pore size.

In an eighth embodiment, the composition according to the seventh embodiment, wherein the first silica-matrix encapsulated biomaterial has a larger average pore size than the second silica-matrix encapsulated biomaterial.

In a ninth embodiment the composition according to any one of the first to seventh embodiments, wherein the first biomaterial is the same as the second biomaterial.

In a tenth embodiment the composition according to any one of the first to ninth embodiments further including at least one additional silica-matrix encapsulated biomaterial that forms at least one additional layer, wherein the third silica-matrix encapsulated biomaterial may optionally have at least one property that is different than that of either the first or second silica-matrix encapsulated biomaterials.

In an eleventh embodiment, a method of making a silica-matrix encapsulated biomaterial for adsorbing and biodegrading at least one target component, the method including determining a desired level of hydrophobicity of the silica-matrix encapsulated biomaterial, the desired level of hydrophobicity being based on the target component; selecting at least a first and a second silica matrix precursor, wherein one of the first and second silica matrix precursor is more hydrophobic than the other; and forming a silica-matrix encapsulated biomaterial from at least the first and second silica matrix precursors.

In a twelfth embodiment, a method of degrading at least one target component, the method including contacting a medium containing the at least one target component and at least one hydrophobic silica-matrix encapsulated biomaterial, the at least one hydrophobic silica-matrix encapsulated biomaterial including a silica matrix and at least one biomaterial, wherein the silica matrix is formed from at least one hydrocarbon moiety containing compound and at least one bridging oxygen moiety containing compound, wherein the target component is degraded by the biomaterial in the at least one hydrophobic silica-matrix encapsulated biomaterial at a rate that is higher than the target component would be degraded by the biomaterial in a silica-matrix encapsulated biomaterial formed without the at least one hydrocarbon moiety containing compound.

In a thirteenth embodiment, the method according to the twelfth embodiment, wherein the hydrocarbon moiety containing compound is selected from methyltrimethyoxysilane (MTMS), triethoxy-methylsilane (TeMs), triethoxy-vinylsilane (TeVs), triethoxy-phenylsilane (TePs), and combinations thereof.

In a fourteenth embodiment, the method according to the twelfth embodiment, wherein the bridging oxygen moiety containing compound is selected from: tetramethyl orthosilicate (TMOS), tetraethyl orthosilicate (TEOS), tetrakis(2-hydroxytehyl) orthosilicate, methydiethyloxysilane, tetrakis(2-hydroxyethyl)orthosilicate (THEOS), 3-(glycidoxypropyl)triethoxysilane (GPMS), 3-(trimethoxy silyl)propylacrylate (TMSPA), N-(3-triethyoxysilylpropyl)pyrrole (TESPP), vinyltriethoxysilane (VTES), methacryloxypropyltriethoxysilane (TESPM), silica nanoparticles (e.g. Ludox or Nyacol), sodium silicate, diglycerylsilane, 3-(2,4-dinitrophenylamino)propyltriethoxysilane, mercaptopropyltriethoxysilane (TEPMS), isocyanotopropyltriethoxysilane, triethoxysilyl-terminated poly(oxypropylene), and combinations thereof.

In a fifteenth embodiment, a silica-matrix encapsulated biomaterial forming composition that includes at least one amine group containing silica precursor; and at least one biomaterial.

In a sixteenth embodiment, the silica-matrix encapsulated biomaterial forming composition according to the fifteenth embodiment, wherein the composition further includes a bridging oxygen moiety containing silica precursor.

In a seventeenth embodiment, the silica-matrix encapsulated biomaterial forming composition according to the fifteenth embodiment, wherein the amine group containing silica precursor is selected from: 3-aminopropyltriethoxysilane (APTS), 3-(2-aminoethylamino)propyltriethyoxysilane, or combinations thereof.

In an eighteenth embodiment, the silica-matrix encapsulated biomaterial forming composition according to the fifteenth embodiment, wherein the amine group containing silica precursor is 3-aminopropyltriethoxysilane (APTS).

In a nineteenth embodiment, a silica-matrix encapsulated biomaterial formed from any one of the compositions according to embodiments fifteenth to eighteenth.

In a twentieth embodiment, the silica-matrix encapsulated biomaterial according to the nineteenth embodiment, wherein degradation of a target component is increased compared to a silica-matrix encapsulated biomaterial formed without the amine group containing silica precursor.

One skilled in the art will appreciate that the articles, devices and methods described herein can be practiced with embodiments other than those disclosed. The disclosed embodiments are presented for purposes of illustration and not limitation. One will also understand that components of the articles, devices and methods depicted and described with regard to the figures and embodiments herein may be interchangeable.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise. The term “and/or” means one or all of the listed elements or a combination of any two or more of the listed elements.

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising” and the like. For example, a conductive trace that “comprises” silver may be a conductive trace that “consists of” silver or that “consists essentially of” silver.

As used herein, “consisting essentially of,” as it relates to a composition, apparatus, system, method or the like, means that the components of the composition, apparatus, system, method or the like are limited to the enumerated components and any other components that do not materially affect the basic and novel characteristic(s) of the composition, apparatus, system, method or the like.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

Also herein, the recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” a particular value, that value is included within the range.

Use of “first,” “second,” etc. in the description above and the claims that follow is not intended to necessarily indicate that the enumerated number of objects are present. For example, a “second” substrate is merely intended to differentiate from another infusion device (such as a “first” substrate). Use of “first,” “second,” etc. in the description above and the claims that follow is also not necessarily intended to indicate that one comes earlier in time than the other.

1. A composition comprising:
a first silica-matrix encapsulated biomaterial, the first silica-matrix encapsulated biomaterial comprising a first silica matrix and a first biomaterial; and
a second silica-matrix encapsulated biomaterial, the second silica-matrix encapsulated biomaterial comprising a second silica matrix and a second biomaterial,
wherein the first silica-matrix encapsulated biomaterial has at least one property that is different than that of the second silica-matrix encapsulated biomaterial, and
wherein the first silica-matrix encapsulated biomaterial forms a first layer and the second silica-matrix encapsulated biomaterial forms a second layer, and the first layer is positioned adjacent the second layer.

2. The composition according to claim 1, wherein the at least one property can be chosen from the porosity, the permeability, the surface charge, the surface functionality, the average pore size, the surface energy, and the chemical composition.

3. The composition according to claim 1, wherein the at least one property is surface energy.

4. The composition according to claim 3, wherein the first silica-matrix encapsulated biomaterial is more hydrophobic than the second silica-matrix encapsulated biomaterial.

5. The composition according to claim 1, wherein the at least one property is porosity.

6. The composition according to claim 5, wherein the first silica-matrix encapsulated biomaterial is more porous than the second silica silica-matrix encapsulated biomaterial.

7. The composition according to claim 1, wherein the at least one property is average pore size.

8. The composition according to claim 7, wherein the first silica-matrix encapsulated biomaterial has a larger average pore size than the second silica-matrix encapsulated biomaterial.

9. The composition according to claim 1, wherein the first biomaterial is the same as the second biomaterial.

10. The composition according to claim 1 further comprising at least one additional silica-matrix encapsulated biomaterial that forms at least one additional layer, wherein the third silica-matrix encapsulated biomaterial may optionally have at least one property that is different than that of either the first or second silica-matrix encapsulated biomaterials.

11. The composition according to claim 1, wherein the composition is made by:
determining a desired level of hydrophobicity of the silica-matrix encapsulated biomaterial, the desired level of hydrophobicity being based on the target component;
selecting at least a first and a second silica matrix precursor, wherein one of the first and second silica matrix precursor is more hydrophobic than the other; and
forming a silica-matrix encapsulated biomaterial from at least the first and second silica matrix precursors.

12. A method of degrading at least one target component, the method comprising:
contacting a medium containing the at least one target component and at least one hydrophobic silica-matrix encapsulated biomaterial, the at least one hydrophobic silica-matrix encapsulated biomaterial comprising a silica matrix and at least one biomaterial, wherein the silica matrix is formed from at least one hydrocarbon moiety containing compound and at least one bridging oxygen moiety containing compound,
wherein the target component is degraded by the biomaterial in the at least one hydrophobic silica-matrix encapsulated biomaterial at a rate that is higher than the target component would be degraded by the biomaterial in a silica-matrix encapsulated biomaterial formed without the at least one hydrocarbon moiety containing compound.

13. The method according to claim 12, wherein the hydrocarbon moiety containing compound is selected from methyltrimethyoxysilane (MTMS), triethoxy-methylsilane (TeMs), triethoxy-vinylsilane (TeVs), triethoxy-phenylsilane (TePs), and combinations thereof.

14. The method according to claim 12, wherein the bridging oxygen moiety containing compound is selected from: tetramethyl orthosilicate (TMOS), tetraethyl orthosilicate (TEOS), tetrakis(2-hydroxytehyl) orthosilicate, methydiethyloxysilane, tetrakis(2-hydroxyethyl)orthosilicate (THEOS), 3-(glycidoxypropyl)triethoxysilane (GPMS), 3-(trimethoxysilyl)propylacrylate (TMSPA), N-(3-triethyoxysilylpropyl)pyrrole (TESPP), vinyltriethoxysilane (VTES), methacryloxypropyltriethoxysilane (TESPM), silica nanoparticles, sodium silicate, diglycerylsilane, 3-(2,4-dinitrophenylamino)propyltriethoxysilane, mercaptopropyltriethoxysilane (TEPMS), isocyanotopropyltriethoxysilane, triethoxysilyl-terminated poly(oxypropylene), and combinations thereof.

15. A silica-matrix encapsulated biomaterial forming composition comprising:
at least one amine group containing silica precursor; and
at least one biomaterial.

16. The silica-matrix encapsulated biomaterial forming composition according to claim 15, wherein the composition further comprises a bridging oxygen moiety containing silica precursor.

17. The silica-matrix encapsulated biomaterial forming composition according to claim 15, wherein the amine group containing silica precursor is selected from: 3-aminopropyltriethoxysilane (APTS), 3-(2-aminoethylamino)propyltriethyoxysilane, or combinations thereof.

18. The silica-matrix encapsulated biomaterial forming composition according to claim 15, wherein the amine group containing silica precursor is 3-aminopropyltriethoxysilane (APTS).

19. A silica-matrix encapsulated biomaterial formed from any one of the compositions according to claim 15.

20. The silica-matrix encapsulated biomaterial according to claim 19, wherein degradation of a target component is increased compared to a silica-matrix encapsulated biomaterial formed without the amine group containing silica precursor.
